SEQUENCE LISTING (79 sequences)

SEQ ID NO: 1 - DNA, 215 bp, Zea mays
ctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctct

SEQ ID NO: 2 - DNA, 1102 bp, Zea mays
gtacctccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 3 - DNA, 1319 bp, Artificial Sequence
  Reverse complement of polynucleotide comprising Z. mays minUbi1P minimal core promoter; Z. mays Ubi1 leader; and Z mays Ubi1 intron
  Ubi1-intron (1)..(1015)
  Ubi1-leader (1016)..(1097)
  minUbi1P-min_core_promoter (1098)..(1319)
ctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcgaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagaggggggggggggggaggacgagcggcgtaccttgaagcggaggtgccgacgggtggatttgggggagatctggttgtgtgtgtgtgcgctccgaacaacacgaggttggggaggtaccaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccag

SEQ ID NO: 4 - DNA, 680 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcct

SEQ ID NO: 5 - DNA, 3322 bp, Artificial Sequence
  Exemplary synthetic Ubi1 bidirectional promoter
  First_minUbi1P-reverse_complement (1105)..(1319)
  Second_minUbi1P-reverse_complement (2009)..(2244)
ctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcgaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagaggggggggggggggaggacgagcggcgtaccttgaagcggaggtgccgacgggtggatttgggggagatctggttgtgtgtgtgtgcgctccgaacaacacgaggttggggaggtaccaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagccgcggagtgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 6 - DNA, 6698 bp, Artificial Sequence
  Exemplary nucleic acid comprising yfp and GUS expression cassettes driven by a synthetic Ubi1 bidirectional promoter
agcacttaaagatctttagaagaaagcaaagcatttattaatacataacaatgtccaggtagcccagctgaattacaatacgcaactgctcataataattcaacaaacccaagtagtacacaacatccagaagcaaataaagcccatacgtaccaaagcctacacaagcagcaacactcactgccagtgccggtgggtctttaaagcacacgggccttgaccacgcgatccaccttgaaacaaacttggtaaaattaaagcaaaccagaagcacacacacgccaacgcaacgcttctgatcgcgcgcccaaggcccggccggccagaacgtacgacggacacgcacacgctgcgaccgagctctaggtgattaagctaactactcaaaggtaggtcttgcgacagtcaacagctctgacagtttctttcaaggacatgttgtctctgtggtctgtcacatctttggaaagtttcacatggtaagacatgtgatgatactctggaacatgaactggacctccaccaatgggagtgttcatctgggtgtggtcagccactatgaagtcgcctttgctgccagtaatctcatgacagatcttgaaggctgacttgagaccgtggttggcttggtcaccccagatgtagaggcagtggggagtgaagttgaactccaagttctttcccaacacatgaccatctttcttgaagccttgaccattgagtttgaccctattgtagacagacccattctcaaaggtgacttcagccctagtcttgaagttgccatctccttcaaaggtgattgtgcgctcttgcacatagccatctggcatacaggacttgtagaagtccttcaactctggaccatacttggcaaagcactgtgctccataggtgagagtggtgacaagtgtgctccaaggcacaggaacatcaccagttgtgcagatgaactgtgcatcaacctttcccactgaggcatctccgtagcctttcccacgtatgctaaaggtgtggccatcaacattcccttccatctccacaacgtaaggaatcttcccatgaaagagaagtgctccagatgccatggtgtcgtgtggatccggtacacacgtgcctaggaccggttcaactaactactgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcgaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatgcatggaccggaacgccgatctagagaaggtagagaggggggggggggggaggacgagcggcgtaccttgaagcggaggtgccgacgggtggatttgggggagatctggttgtgtgtgtgtgcgctccgaacaacacgaggttggggaggtaccaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagccgcggagtgtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaatagtttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattatttcgatcttgatatacttggatgatggcatatgcagcagctatatgtggatttttttagccctgccttcatacgctatttatttgcttggtactgtttcttttgtcgatgctcaccctgttgtttggtgttacttctgcaggtacagtagttagttgaggtacagcggccgcagggcaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagacgtccggtaacctttaaactgagggcactgaagtcgcttgatgtgctgaattgtttgtgatgttggtggcgtattttgtttaaataagtaagcatggctgtgattttatcatatgatcgatctttggggttttatttaacacattgtaaaatgtgtatctattaataactcaatgtataagatgtgttcattcttcggttgccatagatctgcttatttgacctgtgatgttttgactccaaaaaccaaaatcacaactcaataaactcatggaatatgtccacctgtttcttgaagagttcatctaccattccagttggcatttatcagtgttgcagcggcgctgtgctttgtaacataacaattgttacggcatatatccaa

SEQ ID NO: 7 - DNA, 19 bp, Artificial Sequence
  YFP Forward primer
gatgcctcagtgggaaagg

SEQ ID NO: 8 - DNA, 22 bp, Artificial Sequence
  YFP Reverse primer
ccataggtgagagtggtgacaa

SEQ ID NO: 9 - DNA, 18 bp, Artificial Sequence
  Invertase forward primer
tggcggacgacgacttgt

SEQ ID NO: 10 - DNA, 19 bp, Artificial Sequence
  Invertase Reverse primer
aaagtttggaggctgccgt

SEQ ID NO: 11 - DNA, 26 bp, Artificial Sequence
  Invertase probe
cgagcagaccgccgtgtacttctacc

SEQ ID NO: 12 - DNA, 20 bp, Artificial Sequence
  AAD1 Forward primer
tgttcggttccctctaccaa

SEQ ID NO: 13 - DNA, 22 bp, Artificial Sequence
  AAD1 Reverse primer
caacatccatcaccttgactga

SEQ ID NO: 14 - DNA, 24 bp, Artificial Sequence
  AAD1 probe
cacagaaccgtcgcttcagcaaca

SEQ ID NO: 15 - DNA, 215 bp, Artificial Sequence
  min-Ubi1P or Ubi1-min P Minimal core promoter
ctggacccctctcgagtgttccgcttcaccgttggacttgctacgctgtcagcatcgagatgttgcgtggcggagcggcagacttgagccgtcacggcaggcggcctcctcctcctctcacggcatctgtagctacgggggattcctttcgcaccgctcgttcgctttcccttcctcgtctgccgaaataatgttacaccccctccacagcctct

SEQ ID NO: 16 - DNA, 215 bp, Artificial Sequence
  min-Ubi1P or Ubi1-min P Minimal core promoter 2
ctggacccctctcgagagttccgctccaccgttggactagctctgctgtcggcatccagaaaatgcttggcagtgcggcagacgtgagccggcacggcagggggcctcctcctgctctcacggcacatgaagctacgggtgatagcttgcccaccgctccaacgctttcccttactctcacgccgtaataaatagacaccccttccacaacctct

SEQ ID NO: 17 - DNA, 215 bp, Artificial Sequence
  min-Ubi1P or Ubi1-min P Minimal core promoter 3
ctggacctctctcgagagttgcgctccaccgatggacttgctccgctgtcggcgtccataatttgcgtggcggagcggcagacgggagccggcacggcagggagcctcgtcctcctctcacggcacctgcaactacgggggattcctatcccaccgctccttcgctttcacttcttcgccctccttaataagtagacaccccatccgagccctct

SEQ ID NO: 18 - DNA, 215 bp, Artificial Sequence
  min-Ubi1P or Ubi1-min P Minimal core promoter 4
caagacccctctcgagagttccgcaccaccgttggacgtgctccgctatctgcatccagaaattgcgtggcggaacggtaaacgtgagccgtcacggcaggcggcctcctcctcctctcacgacaccggcagctacgggggatacctgtcacacagctccttcgcttttctttcctcgcccgccgtaatatgtatacactccctccgcaccctct

SEQ ID NO: 19 - DNA, 215 bp, Artificial Sequence
  min-Ubi1P or Ubi1-min P Minimal core promoter 5
ctggacccctctcgagggttccgttccaccgttggtcttggtccgctgtcgggatccagaaatagcgtggcggagcggcagacgtgatccggcacggcatgcggcctcctagtcctatcacagcaccggcagctatgggagattccattcccaccgctcctgcgctttcactggctggcccgccgtgatagatagacaccccctccacaccctct

SEQ ID NO: 20 - DNA, 215 bp, Artificial Sequence
  min-Ubi1P or Ubi1-min P Minimal core promoter 6
gttggcttctcttgtgagttctgcttcacggatggacttggtcaacggacggcatccagaatttgcgtggcgtagcggcggacgtgatccggcgcggcaggcggcttcctcctcctctcacttaagcgacagctacaggggattcctttcccaccgctccttcgcttgccgtacctcgcccgccgtaataaatagacaccccttccactccctct

SEQ ID NO: 21 - DNA, 215 bp, Artificial Sequence
  min-Ubi1P or Ubi1-min P Minimal core promoter 7
ctggatccctctcgagagtgcggctccgacgttggacttgctccgaagtcggcatccaaaaattgcgtggtggagaggcagacttgagccggcacggcaggaggcctcgtcctactcgcacggtatcggcagcaacgggagaatccttgcactctgctccttcgctgtaccttcctcgcccgctgatattgatagacaccccctgcataccctct

SEQ ID NO: 22 - DNA, 215 bp, Artificial Sequence
  min-Ubi1P or Ubi1-min P Minimal core promoter 8
atggacccttctcgagtgttcggctccaccgttagacttgctccacgatcgacatcaagaaattgcgagacggagctacaaacgtaagaaatctcggtagggggcctcctcctcctctcacggcaccggcagctacgggggattcctgtcccacctctccttcacgttccctacctcgcccgccataattaataagcaccccctccgcaccctct

SEQ ID NO: 23 - DNA, 215 bp, Artificial Sequence
  min-Ubi1P or Ubi1-min P Minimal core promoter 9
ctggacccctctaaagagttccacgccaccgttataatggctccgctgtcggcatccagaaattacttggcggatcagcagacgtgagccagcatggctggcggcctcctcctcctctcacgatgccgtcagctacgggggattcctttcccaacgctccttcgctttcctatgcgcgcctgccggattaaataggcagcttctcgtcaccctct

SEQ ID NO: 24 - DNA, 215 bp, Artificial Sequence
  min-Ubi1P or Ubi1-min P Minimal core promoter 10
caagacacctctcgattgttccgcttcaccgttggactttctcctcagtcggcatacagaaattgcttggcgaagcggcagacatgagccggcacgacatgcgtcctcattctcctctcatggcaccggcagttactggtgaatcctatcgcaccgctccttcgctgtcccttaatcgcccgccgaaaataattgacaccccatccacaccctct

SEQ ID NO: 25 - DNA, 215 bp, Artificial Sequence
  min-Ubi1P or Ubi1-min P Minimal core promoter 11
gaggacccctctcgtgtgtatcgctccacctttggagttggtccactatcggcgtacagaaaattcgttgcgaagcggcagacgtgagcctacacggcagtcggcctctacctcctgacaaggcacgtgcagctacagatgatgcctttcccaccactccttcgcgttcctttcctcgccatcagtaatgaatggacacgtcctccagactctct

SEQ ID NO: 26 - DNA, 215 bp, Artificial Sequence
  min-Ubi1P or Ubi1-min P Minimal core promoter 12
ctgaacccatctcgagtatgccgcacgatcgattgacatgctccactggcagcatccagaaattgcattggggagcatcaggcgtgagcctgcacggcaggcggactattcctcctcgcgcggcaccggcaactacgggggatgcttgaccgaccgctccatcgatttcccaatctcgcttgccgtattaaatagataaccccttcacaccctct

SEQ ID NO: 27 - DNA, 215 bp, Artificial Sequence
  min-Ubi1P or Ubi1-min P Minimal core promoter 13
ctggactccttacgggagatccgctccaccgttggactagctccgttttcggcttcaataaagggcgtgggggagcggcagtcgggggcaggcacggcagtggtcctcatccatatctcacggggccggcagttgagggggattcctgtcccacctcacctactctttccctacctcgtctgccatattaaatagtcaccccctccacaaccttt

SEQ ID NO: 28 - DNA, 215 bp, Artificial Sequence
  min-Ubi1P or Ubi1-min P Minimal core promoter 14
ttggacccctctcgaaagttaggctccgccgttggactggtttcgcggtcatcaatcaggaattgcggggcggagggtcagacgtgtgccggcacagcaggtggcctcctcatcgtcacaaggcactggcaactacgggtgattcatttccttcagcacctacgcttaccctgccacgccctccgtattataatgacaccccctccacaccttat

SEQ ID NO: 29 - DNA, 215 bp, Artificial Sequence
  min-Ubi1P or Ubi1-min P Minimal core promoter 15
ctggaccccacgcggggttttcgttcctccgttgggatagctccggtgtcagcatacagagaatatatgtcggagcggaagacgtgagccgacacggcgggctgccgcctcctcctgtcacgacaccggcaggtacgggggattccgttcccgccgcacagtcactttcgcttccttgccggtcgtattaaatagacaccgtgtccacagcctct

SEQ ID NO: 30 - DNA, 215 bp, Artificial Sequence
  min-Ubi1P or Ubi1-min P Minimal core promoter 16
cttgagcccactctagagttccgtttcaccgaatgactagctccgctgtcggtatccattaagtgggaggcagaacgtcatatgagagtcggcacgggaggcgttcgccacgtccgcgcactacagcgggagctgcggaatatacctgtcccaatgctgctacgctttcccttccgcgcccaccgtagaaaaatgacagtcccttcacaccctct

SEQ ID NO: 31 - DNA, 215 bp, Artificial Sequence
  min-Ubi1P or Ubi1-min P Minimal core promoter 17
taggaggcctctcgaaaggtccggaactccgtaggacgtgctccgctgacagcatccaggaatatcatgggggagctgcagacgagagcctggacgacaaggggtcacctcggccgctgacagctgcggcagcaacggagtatgcttttctcaccgctccggcgctttcccttcgacgcaggccagaataagtagacatcagcgccacaccctct

SEQ ID NO: 32 - DNA, 215 bp, Artificial Sequence
  min-Ubi1P or Ubi1-min P Minimal core promoter 18
cttgtctccactctgatgttccgctccaacatttgatttgctcctctgtaggcatacagttattgggggactgatcggcagacgtgagccagcactgcaaacggccaactcctcctctctcgactaagggattaattaaggataccttacccgcggctccttctctttccctacctagcccgccttattaaatagagaccgcctccacagccgct

SEQ ID NO: 33 - DNA, 215 bp, Artificial Sequence
  min-Ubi1P or Ubi1-min P Minimal core promoter 19
ctgtacccttcacaagggttacacgctaccgatggacttgcaccactgtggggttccaataattgcgtggctgggcgtcagacatattccggcatggcaagcggcctgctcctcctctgggagcaccggcaacaatgggggattccaagcccgcaggtccttcgttttaccgtcctcgcccgccgtagtatgtaggcatcccagagactacctct

SEQ ID NO: 34 - DNA, 215 bp, Artificial Sequence
  min-Ubi1P or Ubi1-min P Minimal core promoter 20
caggaaccctaacgagggttccgcacgaccaaatgacttgatcttctgtcggcatccagaaatggggtgtcagagcggcatgcgtgagccggcggggcgtgcggcctcatgctgctctcgcgggactaggagttacgggggatacctgtattgccgctccgacactgtaccatcctctcccgccggagtatagagacaccccctcgacgccatat

SEQ ID NO: 35 - DNA, 215 bp, Artificial Sequence
  min-Ubi1P or Ubi1-min P Minimal core promoter 21
ctgtgctcctgtatggggttcaactccaccgtgaaatttgcgcctctgtcgtcatccagaaattgcgtggttgatctgctgacgttaaagggctctgcaggcggcttccttcggctatgaaggtactggcgtctgcaagtgatgcttttgctaactcgccttcgatgtcccttcctcgcgtgctttaataggttgtcagccgctccagaccattt

SEQ ID NO: 36 - DNA, 215 bp, Artificial Sequence
  min-Ubi1P or Ubi1-min P Minimal core promoter 22
ctggtcccatcgctagtggtacgctccaccggtggagtagctcagatgtctgaagggtggaatttagaggtggagagacagacgtgagctagagcggcatgggacctggtccaccgctcgaggcaatggcaacgactgttgaaaccttgcccaccactcctgcaattttccatcctcaccggccggaatgaattaaaacccacgtcacaacctct

SEQ ID NO: 37 - DNA, 215 bp, Artificial Sequence
  min-Ubi1P or Ubi1-min P Minimal core promoter 23
cgtgacagggctcgggtgttcggctccatcgtagtgcatgcgccgatgtaagtatacaagaagtacgtggcttggcgtctgacgagggccgtcaaggcaggcggcctccttctaagcttacggcgccggcaggttcgtaggttaccttacactcaactcatagtctatctattactcgtactgcgttataaattgtcaccccctccacaccctct

SEQ ID NO: 38 - DNA, 215 bp, Artificial Sequence
  min-Ubi1P or Ubi1-min P Minimal core promoter 24
aggaacgcttctcgatggttgcgcacataggagggacttgatagtcggtggaaatctaagaattgcatatcagatctgcagacgttagccgacatggctagcagactactccgcttcacacgtcagcgaaagcgacggaggatttcttgccaacggcgccttcgcgaacccttcctcgcccgtcggaagaaagatactccccttgcacaccctct

SEQ ID NO: 39 - DNA, 215 bp, Artificial Sequence
  min-Ubi1P or Ubi1-min P Minimal core promoter 25
cttgacttggctcgagagttctgcgcttccattgtagttgcagcgatgtcggagtccgagggttgcgtggcggtgcggcagacgtgggcagatacgactgtatgccagcacctaaacatacggtaccagaagctgcggtggatacctttcccgacgcatatacgttttccgtgcctctcacgccgtagtaaataaactccccctcctgttccttt

SEQ ID NO: 40 - DNA, 8 bp, Artificial Sequence
  YFP probe
cttggagc

SEQ ID NO: 41 - DNA, 20 bp, Artificial Sequence
  Cry34 Forward Primer
gccaacgaccagatcaagac

SEQ ID NO: 42 - DNA, 23 bp, Artificial Sequence
  Cry34 Reverse Primer
gccgttgatggagtagtagatgg

SEQ ID NO: 43 - DNA, 18 bp, Artificial Sequence
  Cry34 Probe
ccgaatccaacggcttca

SEQ ID NO: 44 - DNA, 17 bp, Artificial Sequence
  Cry35 Forward Primer
cctcatccgcctcaccg

SEQ ID NO: 45 - DNA, 22 bp, Artificial Sequence
  Cry35 Reverse Primer
ggtagtccttgagcttggtgtc

SEQ ID NO: 46 - DNA, 19 bp, Artificial Sequence
  Cry35 Probe
cagcaatggaacctgacgt

SEQ ID NO: 47 - DNA, 29 bp, Artificial Sequence
  PAT Forward Primer
acaagagtggattgatgatctagagaggt

SEQ ID NO: 48 - DNA, 29 bp, Artificial Sequence
  PAT Reverse Primer
ctttgatgcctatgtgacacgtaaacagt

SEQ ID NO: 49 - DNA, 29 bp, Artificial Sequence
  PAT Probe
ggtgttgtggctggtattgcttacgctgg

SEQ ID NO: 50 - protein, 234 aa, Phialidium sp.
MSSGALLFHGKIPYVVEMEGNVDGHTFSIRGKGYGDASVGKVDAQFICTTGDVPVPWSTLVTTLTYGAQCFAKYGPELKDFYKSCMPEGYVQERTITFEGDGVFKTRAEVTFENGSVYNRVKLNGQGFKKDGHVLGKNLEFNFTPHCLYIWGDQANHGLKSAFKIMHEITGSKEDFIVADHTQMNTPIGGGPVHVPEYHHITYHVTLSKDVTDHRDNMSLVETVRAVDCRKTYL

SEQ ID NO: 51 - protein, 234 aa, Phialidium sp.
MSSGALLFHGKIPYVVEMEGNVDGHTFSIRGKGYGDASVGKVDAQFICTTGDVPVPWSTLVTTLTYGAQCFAKYGPELKDFYKSCMPDGYVQERTITFEGDGNFKTRAEVTFENGSVYNRVKLNGQGFKKDGHVLGKNLEFNFTPHCLYIWGDQANHGLKSAFKICHEITGSKGDFIVADHTQMNTPIGGGPVHVPEYHHMSYHVKLSKDVTDHRDNMSLKETVRAVDCRKTYL

SEQ ID NO: 52 - protein, 76 aa, Artificial Sequence
  Ubi polypeptide
MQIFVKTLTGKTITLEVESSDTIDNVKAKIQDKEGIPPDQQRLIFAGRQLEDGRTLADYNIQKESTLHLVLRLRGG

SEQ ID NO: 53 - protein, 103 aa, Artificial Sequence
  SUMO polypeptide
GSMSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGGHSTV

SEQ ID NO: 54 - protein, 63 aa, Artificial Sequence
  FMDV 2A
GSGSRVTELLYRMKRAETYCPRPLLAIHPTEARHKQKIVAPVKQLLNFDLLKLAGDVESNPGP

SEQ ID NO: 55 - protein, 25 aa, Strongylocentrotus purpuratus
DGFCILYLLLILLMRSGDVETNPGP

SEQ ID NO: 56 - DNA, 585 bp, Artificial Sequence
  IRES AM403095.1
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 57 - DNA, 596 bp, Artificial Sequence
  IRES GQ357182.1
gaattccgccccccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataata

SEQ ID NO: 58 - DNA, 575 bp, Artificial Sequence
  IRES KC710227.1
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctggccccccgaaccacggggacgtggttttcctttgaaaaacacgatgataatatggccacaacc

SEQ ID NO: 59 - DNA, 576 bp, Artificial Sequence
  IRES KC710228.1
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 60 - DNA, 596 bp, Artificial Sequence; identical to SEQ ID NO: 57
  IRES EU916835.1

SEQ ID NO: 61 - DNA, 594 bp, Artificial Sequence
  IRES KJ667592.1
gccccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 62 - protein, 159 aa, Artificial Sequence
  Ssp intein
CLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIKMVKVIGRRSLGVQRIFDIGLPQDHNFLLANGAIAAN

SEQ ID NO: 63 - protein, 201 aa, Artificial Sequence
  gi|64174789|gb|AAY41168.1|
MGSSHHHHHHSSGLVPRGSHMQYKLILNGKTLKGETTTEAVDAATAEKVFKQYANDNGVDGEWTYDDATKTYTVTEGSCLSFGTEILTVEYGPLPIGKIVSEEINCSVYSVDPEGRVYTQAIAQWHDRGEQEVLEYELEDGSVIRATSDHRFLTTDYQLLAIEEIFARQLDLLTLENIKQTEEALDNHRLPFPLLDAGTIK

SEQ ID NO: 64 - protein, 108 aa, Artificial Sequence
  gi|659835300
  misc_feature (81)..(81): Xaa can be any naturally occurring amino acid
  misc_feature (87)..(87): Xaa can be any naturally occurring amino acid
  misc_feature (101)..(101): Xaa can be any naturally occurring amino acid
HHHHHHCLSYETEILTVEYGLLPIGKIVEKRIECTVYSVDNNGNIYTQPVAQWHDRGEQEVFEYCLEDGSLIRATKDHKFXTVDGQXLPIDEIFERELDLXRVDNLPN

SEQ ID NO: 65 - protein, 113 aa, Artificial Sequence
  gi|543516928|emb|CCQ50212.1|
MIKFAEYCLSYDTEILTVEYGAMYIGKIVEENINCTVYTVDKNGFVYTQTIAQWHNRGEQEIFEYDLEDGSKIKATKDHKFMTIDGEMLPIDEIFEKNLDLKQVVSHPDDYLV

SEQ ID NO: 66 - protein, 82 aa, Artificial Sequence
  gb|ABA21226.1|
CLSYDTEVLTVEYGFVPIGEIVDKGIECSVFSIDSNGIVYTQPIAQWHHRGKQEVFEYCLEDGSIIKATKDHKFMTQDGKML

SEQ ID NO: 67 - protein, 110 aa, Artificial Sequence
  gb|AAP47638.1|
CLSYDTEIWTVEYGAMPIGKIVEEKIECSVYTVDENGFVYTQPIAQWHPRGQQEIIEYTLEDGRKIRATKDHKMMTESGEMLPIEEIFQRELDLKVETFHEMSLLRRGAK

SEQ ID NO: 68 - protein, 77 aa, Artificial Sequence
  gi|605045185
CLSYDTQILTVEYGLIPIGEIVEKGIECQVYTVNKSGNVYTQPIAQWHYRGEQEIFEYDLEDGSVIRATKDHKFMTT

SEQ ID NO: 69 - protein, 82 aa, Artificial Sequence
  gb|AIS22787.1
CLSYETEILTVEYGSLPIGKIVEKRIECTVYSVDNNGNIYTQPVAQWHDRGEQEVFEYCLEDGSLIRATKDHKFMTVDGQML

SEQ ID NO: 70 - protein, 111 aa, Artificial Sequence
  gi|823631048
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNLEGHHHHHH

SEQ ID NO: 71 - protein, 137 aa, Artificial Sequence
  gi|37784578|gb|AAP47640.1|
GATKNGVPQETAEGLFEQMVKFAEYCLSYNTEVLTVEYGPLPIGKIVDEQIHCRVYSVDENGFVYTQAIAQWHDRGYQEIFAYELADGSVIRATKDHQFMTEDGQMFPIDEIWEKGLDLKKLPTVQDLPAAVGYTVS

SEQ ID NO: 72 - protein, 137 aa, Artificial Sequence
  gi|538261247|pdb|4KL6|
SGGALSYETEILTVEYGLLPIGKIVEKRIECTVYSVDNNGNIYTQPVAQWHDRGEQEVFEYCLEDGSLIRATKDHKFMVDGQMLPIDEIFERELDLMRNPGIKIATRKYLGKQNVYDIGVERDHNFALKNGFIASNA

SEQ ID NO: 73 - protein, 139 aa, Artificial Sequence
  gi|237823419|pdb|2KEQ|
GGALSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNIKIATRKYLGKQNVYDIGVERDHNFALKNGFIASN

SEQ ID NO: 74 - protein, 144 aa, Artificial Sequence
  gi|538261245|pdb|4KL5|
SGGALSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNIKIATRKYLGKQNVYDIGVERDHNFALKNGFIASNADNG

SEQ ID NO: 75 - protein, 91 aa, Artificial Sequence
  gb|AIE73590.1|
CLSFDAEILTVEYGPLSIGKIVGEEINCSVYSVDPQGRIYTQAIAQWHDRGVQEVFEYELEDGSVIRATPDHRFLTTDYELLAIEEIFARQ

SEQ ID NO: 76 - protein, 108 aa, Artificial Sequence; identical to SEQ ID NO: 64
  gi|659835300|
  misc_feature (81)..(81): Xaa can be any naturally occurring amino acid
  misc_feature (87)..(87): Xaa can be any naturally occurring amino acid
  misc_feature (101)..(101): Xaa can be any naturally occurring amino acid

SEQ ID NO: 77 - protein, 113 aa, Artificial Sequence; identical to SEQ ID NO: 65
  gi|543516928|emb|CCQ50212.1|

SEQ ID NO: 78 - protein, 33 aa, Artificial Sequence
  Linker
LNLAEEAAAKEAAAKEAAAKEAAAKEAAAKAAA

SEQ ID NO: 79 - protein, 5 aa, Artificial Sequence
  Linker
EEKKN

(19) United States
(12) Patent Application Publication
(10) Pub. No.: US 2017/0029833 A1
(43) Pub. Date: Feb. 2, 2017

(54) CONSTRUCT AND METHOD FOR SYNTHETIC BIDIRECTIONAL PLANT PROMOTER UBI1

(71) Applicant: Dow AgroSciences LLC, Indianapolis, IN (US)

(72) Inventors: Sandeep Kumar, Carmel, IN (US); Diaa Alabed, Carmel, IN (US); Terry R. Wright, Carmel, IN (US); Manju Gupta, Carmel, IN (US)

(21) Appl. No.: 15/212,000

(22) Filed: Jul. 15, 2016

Related U.S. Application Data
(63) Continuation-in-part of application No. 13/674,606, filed on Nov. 12, 2012, now Pat. No. 9,422,569.
(60) Provisional application No. 61/582,138, filed on Dec. 30, 2011.
(60) Provisional application No. 61/617,252, filed on Mar. 29, 2012.

(51) Int. Cl. C12N 15/82; C07K 14/005

(52) U.S. Cl. CPC C12N 15/8216; C12N 15/8241; C07K 14/005; C12N 2770/00022

(57) ABSTRACT

Provided are constructs and methods for expressing multiple genes in plant cells and/or plant tissues. The constructs provided comprise at least one bidirectional promoter link to multiple gene expression cassettes. In some embodiments, the constructs and methods provided employ a bidirectional promoter based on a minimal core promoter element from a Zea mays Ubiquitin-1 aerie or a functional equivalent thereof. In some embodiments, the constructs and methods provided allow expression of genes between three and twenty.

### CROSS-REFERENCE TO RELATED APPLICATIONS

This application is a continuation-in-part7-1 of U.S. patent application Ser. No. 13/674,606 filed on Nov. 12, 2012, which claims priority under 35 U.S.C. §119 of U.S. provisional patent application Ser. No. 61/582,138 filed Dec. 30, 2011, and also claims priority of U.S. provisional patent application Ser. No. 61/617,252 filed Mar. 29, 2012, which applications are hereby incorporated by reference in their entirely.

### TECHNICAL FIELD

This invention is generally related to the field of plant molecular biology, and more specifically the field of stable expression of multiple genes in transgenic plants.

### BACKGROUND

Many plant species are capable of being transformed with transgenes from other species to introduce agronomically desirable traits or characteristics, for example, improving nutritional value quality, increasing yield, conferring pest or disease resistance, increasing drought and stress tolerance, improving horticultural qualities (such as pigmentation and growth), imparting herbicide resistance, enabling the production of industrially useful compounds and/or materials from the plant, and/or enabling the production of pharmaceuticals. The introduction of transgenes into plant cells and the subsequent recovery of fertile transgenic plants that contain a stably integrated copy of the transgene can be used to produce transgenic plants that possess the desirable traits.

Control and regulation of gene expression can occur through numerous mechanisms. Transcription initiation of a gene is a predominant controlling mechanism of gene expression. Initiation of transcription is generally controlled by poly/nucleotide sequences located in the 5′-flanking or upstream region of the transcribed gene. These sequences are collectively referred to as promoters. Promoters generally contain signals for RNA polymerase to begin transcription so that messenger RNA (mRNA) can be produced. Mature mRNA is translated by ribosome, thereby synthesizing proteins. DNA-binding proteins interact specifically/ with promoter DNA sequences to promote the formation of a transcriptional complex and initiate the gene expression process. There are a variety of eukaryotic promoters isolated and characterized from plants that are functional for driving the expression of a transgene in plants. Promoters that affect gene expression in response to environmental stimuli, nutrient availability, or adverse conditions including heat shock, anaerobiosis, or the presence of heavy metals have been isolated and characterized. There are also promoters that control gene expression during development or in a tissue, or organ specific fashion. In addition, prokaryotic promoters isolated from bacteria and virus have been isolated and characterized that are functional for driving the expression of a transgene in plants.

A typical eukaryotic promoter consists of a minimal promoter and other cis-elements. The minimal promoter is essentially a TATA box region where RNA polymerase II (polII), TATA-binding protein (TBP), and TBP-associated factors (TARs) may bind to initiate transcription. However in most instances, sequence elements other than the TATA motif are required for accurate transcription. Such sequence elements (e.g., enhancers) have been found to elevate the overall level of expression of the nearby genes, often in a position- and/or orientation-independent manner. Other sequences near the transcription start site (e.g., INR sequences) of some polII genes may provide an alternate binding site for factors that also contribute to transcriptional activation, even alternatively providing the core promoter binding sites for transcription in promoters that lack functional TATA elements. See e.g., Zenzie-Gregory et al. (1992) J. Biol. Chem. 267: 2823-30.

Other gene regulatory elements include sequences that interact with specific DNA-binding factors. These sequence motifs are sometimes referred to as cis-elements, and are usually position- and orientation-dependent, though they may be found 5′ or 3′ to a gene's coding sequence, or in an intron. Such cis-elements, to which tissue-specific or development-specific transcription factors bind, individually or in combination, may determine the spatiotemporal expression pattern of a promoter at the transcriptional level. The arrangement of upstream cis-elements, followed by a minimal promoter, typically establishes the polarity of a particular promoter. Promoters in plants that have been cloned and widely used for both basic research and biotechnological application are generally unidirectional, directing only one gene that has been fused at its 3′ end (i.e., downstream), See, for example, Xie et at (2001) Nat. Biotechnol. 19(7):677-9; U.S. Pat. No. 6,388,170.

Many cis-elements (or “upstream regulatory sequences”) have been identified in plant promoters. These cis-elements vary widely in the type of control they exert on operably linked genes. Some elements act to increase the transcription of operably-linked genes in response to environmental responses (e.g., temperature, moisture, and wounding). Other cis-elements may respond to developmental cues (e.g., germination, seed maturation, and flowering) or to spatial information (e.g., tissue specificity). See, for example, Langridge et al. (1989) Proc. Natl. Acad. Sci. USA 86:3219-23. The type of control of specific promoter elements is typically an intrinsic quality of the promoter; i.e., a heterologous gene under the control of such a promoter is likely to be expressed according to the control of the native gene from which the promoter element was isolated. These elements also typically may be exchanged with other elements and maintain their characteristic intrinsic control over gene expression.

It is often necessary to introduce multiple genes into plants for metabolic engineering and trait stacking, which genes are frequently controlled by identical or homologous promoters. However, homology-based gene silencing (HBGS) is likely to arise when multiple introduced transgenes have homologous promoters driving them. See e.g., Mol et al. (1989) Plant Mol. Biol. 13:287-94. HBGS has been reported to occur extensively in transgenic plants. See e.g., Vaucheret and Fagard (2001) Trends Genet. 17:29-35. Several mechanisms have been suggested to explain the phenomena of HBGS, all of which include the feature that sequence homology in the promoter triggers cellular recognition mechanisms that result in silencing of the repeated genes. See e.g., Matzke and Matzke (1995) Plant Physiol. 107:679-85; Meyer and Saedler (1996) Ann. Rev. Plant Physiol, Plant Mol. Biol. 47:23-48; Fire (1999) Trends Genet. 15:358-63; Hamilton and Baulcombe (1999) Science 286:950-2; and Steimer et al. (2000) Plant Cell 12:1165-78.

Strategies to avoid HBGS in transgenic plants frequently involve the development of synthetic promoters that are functionally equivalent but have minimal sequence homology. When such synthetic promoters are used for expressing transgenes in crop plants, they may aid in avoiding or reducing HBGS. See e.g., Mourrain et al. (2007) Planta 225(2):365-79; Bhullar et al. (2003) Plant Physiol. 132:988-98. Such promoters can be generated by introducing known cis-elements in a novel or synthetic stretch of DNA, or alternatively by “domain swapping,” wherein domains of one promoter are replaced with functionally equivalent domains from other heterologous promoters.

Thus, there remains a need for constructs and methods for stable expression of multiple transgenes effectively with minimum risk for recombination or loss of transgenes through breeding or multiple generations in transgenic plants.

### DISCLOSURE

Many plant species are capable of being transformed with transgenes from other species to introduce agronomically desirable traits or characteristics, for example, improving nutritional value quality, increasing yield, conferring pest or disease resistance, increasing drought and stress tolerance, improving horticultural qualities (such as pigmentation and growth), imparting herbicide resistance, enabling the production of industrially useful compounds and/or materials from the plant, and/or enabling the production of pharmaceuticals. The introduction of transgenes into plant cells and the subsequent recovery of fertile transgenic plants that contain a stably integrated copy of the transgene can be used to produce transgenic plants that possess the desirable traits.

Control and regulation of gene expression can occur through numerous mechanisms. Transcription initiation of a gene is a predominant controlling mechanism of gene expression. Initiation of transcription is generally controlled by polynucleotide sequences located in the 5′-flanking or upstream region of the transcribed gene. These sequences are collectively referred to as promoters. Promoters generally contain signals for RNA polymerase to begin transcription so that messenger RNA (mRNA) can be produced. Mature mRNA is translated by ribosome, thereby synthesizing proteins. DNA-binding proteins interact specifically with promoter DNA sequences to promote the formation of a transcriptional complex and initiate the gene expression process. There are a variety of eukaryotic promoters isolated and characterized from plants that are functional for driving the expression of a transgene in plants. Promoters that affect gene expression in response to environmental stimuli, nutrient availability, or adverse conditions including heat shock, anaerobiosis, or the presence of heavy metals have been isolated and characterized. There are also promoters that control gene expression during development or in a tissue, or organ specific fashion. In addition, prokaryotic promoters isolated from bacteria and virus have been isolated and characterized that are functional for driving the expression of a transgene in plants.

A typical eukaryotic promoter consists of a minimal promoter and other cis-elements. The minimal promoter is essentially a TATA box region where RNA polymerase II (polII), TATA-binding protein (TBP), and TBP-associated factors (TABs) may bind to initiate transcription.

However in most instances, sequence elements other than the TATA motif are required for accurate transcription. Such sequence elements (e.g., enhancers) have been found to elevate the overall level of expression of the nearby genes, often in a position- and/or orientation-independent manner. Other sequences near the transcription start site (e.g., INR sequences) of some polII genes may provide an alternate binding site for factors that also contribute to transcriptional activation, even alternatively providing the core promoter binding sites for transcription in promoters that lack functional TATA elements, See e.g., Zenzie-Gregory et at (1992) J. Biol. Chem, 267: 2823-30.

Other gene regulatory elements include sequences that interact with specific DNA-binding factors. These sequence motifs are sometimes referred to as cis-elements, and are usually position- and orientation-dependent, though they may be found 5′ or 3′ to a gene's coding sequence, or in an intron. Such cis-elements, to which tissue-specific or development-specific transcription factors bind, individually or in combination, may determine the spatiotemporal expression pattern of a promoter at the transcriptional level. The arrangement of upstream cis-elements, followed by a minimal promoter, typically establishes the polarity of a particular promoter. Promoters in plants that have been cloned and widely used for both basic research and biotechnological application are generally unidirectional, directing only one gene that has been fused at its 3′ end (i.e., downstream). See, for example, Xie et al. (2001) Nat. Biotechnol. 19(7):677-9; U.S. Pat. No. 6,388,170.

Many cis-elements (or “upstream regulatory sequences”) have been identified in plant promoters. These cis-elements vary widely in the type of control they exert on operably linked genes. Some elements act to increase the transcription of operably-linked genes in response to environmental responses (e.g., temperature, moisture, and wounding). Other cis-elements may respond to developmental cues (e.g., germination, seed maturation, and flowering) or to spatial information (e.g., tissue specificity). See, for example, Langridge et al. (1989) Proc. Natl. Acad. Sci. USA 86:3219-23. The type of control of specific promoter elements is typically an intrinsic quality of the promoter; i.e., a heterologous gene under the control of such a promoter is likely to be expressed according to the control of the native gene from which the promoter element was isolated. These elements also typically may be exchanged with other elements and maintain their characteristic intrinsic control over gene expression.

It is often necessary to introduce multiple genes into plants for metabolic engineering and trait stacking, which genes are frequently controlled by identical or homologous promoters. However, homology-based gene silencing (HBGS) is likely to arise when multiple introduced transgenes have homologous promoters driving them. See e.g., Mol et al, (1989) Plant Mol. Biol. 13:287-94. HBGS has been reported to occur extensively in transgenic plants. See e.g., Vaucheret and Fagard (2001) Trends Genet. 17:29-35. Several mechanisms have been suggested to explain the phenomena HBGS all of which include the feature that sequence homology in the promoter triggers cellular recognition mechanisms that result in silencing of the repeated genes. See e.g., Matzke and Matzke (1995) Plant Physiol, 107:679-85; Meyer and Saedler (1996) Ann. Rev. Plant Physiol. Plant Mol. Biol. 47:23-48; Fire (1999) Trends Genet. 15:358-63; Hamilton and Baulcombe (1999) Science 286:950-2; and Steimer et al. (2000) Plant Cell 12:1165-78.

Strategies to avoid HBGS in transgenic plants frequently involve the development of synthetic promoters that are functionally equivalent but have minimal sequence homology. When such synthetic promoters are used for expressing transgenes in crop plants, they may aid in avoiding or reducing HBGS. See e.g., Mourrain et al. (2007) Planta 225(2):365-79; Bhullar et al. (2003) Plant Physiol, 132:988-98. Such promoters can be generated by introducing known cis-elements in a novel or synthetic stretch of DNA, or alternatively by “domain swapping,” wherein domains of one promoter are replaced with functionally equivalent domains from other heterologous promoters.

Thus, there remains a need for constructs and methods for stable expression of multiple transgenes effectively with minimum risk for recombination or loss of transgenes through breeding or multiple generations in transgenic plants.

### SUMMARY OF THE INVENTION

Described herein are methods for converting an Ubi1 polar promoter into synthetic bidirectional promoters, such that one synthetic promoter can direct the expression of two genes flanking the promoter. In some embodiments, a method for converting an Ubi1 polar promoter into a synthetic bidirectional promoter may comprise, for example and without limitation, identifying the minimal promoter element nucleotide sequence of an Ubi1 promoter; and/or providing a nucleic acid comprising two minimal Ubi1 promoter element nucleotide sequences oriented in opposite directions. In particular embodiments, a nucleic acid may comprise two minimal Ubi1 promoter element nucleotide sequences oriented in opposite directions, such that the end of each minimal promoter element that is closest to the corresponding native Ubi1 gene is further from the other minimal promoter element than an end of the nucleic acid that is proximate to a coding sequence operably linked to the promoter element. In some examples, the minimal Ubi1 promoter element is isolated from maize. Additional elements of a native Ubi1 promoter that may be engineered to be included in a synthetic bidirectional promoter include Ubi1 introns, Ubi1 exons, and/or all or part of an Ubi1 upstream promoter region. In some examples, a synthetic bidirectional promoter may comprise more than one of any of the foregoing.

Also described herein are Ubi1 minimal promoters that may be useful in constructing synthetic promoters (e.g., synthetic bidirectional promoters), and particular synthetic promoters produced by the foregoing methods. In some embodiments, a synthetic bidirectional promoter is a promoter that is able to control transcription of an operably linked nucleotide sequence in a plant cell. For example, a synthetic bidirectional promoter may be able in particular embodiments to control transcription in a plant cell of two operably linked nucleotide sequences that flank the promoter.

Particular embodiments of the invention include cells (e.g., plant cells) comprising an Ubi1 minimal promoter or functional equivalent thereof For example, specific embodiments include a cell comprising a synthetic promoter (e.g., a synthetic bidirectional promoter) that includes an Ubi1 minimal promoter or functional equivalent thereof. Plant cells according to particular embodiments may be present in a cell culture, a tissue, a plant part, and/or a whole plant. Thus, a plant (e.g., a monocot or dicot) comprising a cell comprising an Ubi1 minimal promoter or functional equivalent thereof is included in some embodiments.

Some embodiments of the invention include a means for initiating transcription in a direction-independent manner. Means for initiating transcription in a direction-independent manner include the Ubi1 minimal promoter of SEQ NO: 1. Some embodiments of the invention include a means for initiating transcription of two operably linked nucleotide sequences of interest. Means for initiating transcription of two operably linked nucleotide sequences of interest include the synthetic bidirectional promoter of SEQ ID NO: 5.

The foregoing and other features will become more apparent from the following detailed description of several embodiments, which proceeds with reference to the accompanying figures.

Also provided are constructs and methods for expressing multiple genes in plant cells and/or plant tissues. The constructs provided comprise at least one bi-directional promoter link to multiple gene expression cassettes. In some embodiments, the constructs and methods provided employ a bi-directional promoter based on a minimal core promoter element from a Zea mays Ubiquitin-1 gene, or a functional equivalent thereof. In some embodiments, the constructs and methods provided allow expression of genes between three and twenty.

In one aspect, provided is a synthetic polynucleotide comprising a bi-directional promoter comprising:
(a) a first minimal core promoter element selected from SEQ. ID NOs: 1 and 15-39;
(b) a second minimal core promoter element selected from SEQ ID NOs: 1 and 15-39; wherein the two minimal core promoter elements are in reverse complementary orientation with respect to each other in the synthetic polynucleotide; and
(c) a heterologous nucleotide sequence of interest operably linked to the bi-directional promoter.

In one embodiment, the synthetic polynucleotide further comprises a different heterologous nucleotide sequence of interest operably linked to the bi-directional promoter. In another embodiment, the synthetic polynucleotide further comprises an exon from an Ubiquitin-1 gene and an intron from an Ubiquitin-1 gene. In another embodiment, the synthetic polynucleotide further comprises an upstream regulatory sequence from an Ubiquitin-1 gene. In another embodiment, the synthetic polynucleotide further comprises an element selected from the group consisting of an upstream regulatory sequence (URS), an enhancer element, an exon, an intron, a transcription start site, a TATA box, a heat shock consensus element, and combinations thereof.

In another aspect, provided is a method for producing a transgenic cell, the method comprises transforming the cell with the synthetic polynucleotide provided herein. In another aspect, provided is a plant cell comprising the synthetic polynucleotide provided herein.

In another aspect, provided is a nucleic acid construct for expressing multiple genes in plant cells and/or tissues, comprising,
(a) a bi-directional promoter; and
(b) two gene expression cassettes on opposite ends of the bi-directional promoter; wherein at least one of the gene expression cassettes comprises two or more genes linked via a translation switch selected from the group consisting of:
   (i) a polynucleotide sequence coding a 2A peptide or 2A-like peptide selected from SEQ ID NOs: 52-55;
   (ii) an internal ribosome entry site (IRES) selected from SEQ. ID NOs: 56-61;
   (iii) a polynucleotide sequence encoding an intein peptide selected from SEQ ID NOs: 62-77; and
   (iv) a linker peptide selected from SEQ NOs: 78-79.

In one embodiment, the bi-directional promoter comprises:
(a) a first minimal core promoter element selected from SEQ ID NOs: 1 and 15-39; and
(b) a second minimal core promoter element selected from SEQ ID NOs: 1 and 15-39;
   wherein the two minimal core promoter elements are in reverse complementary orientation with respect to each other in the synthetic polynucleotide.

In another aspect, provided is a method for generating a transgenic plant. The method comprises transforming a plant cell with the nucleic acid construct provided herein. In another aspect, provided is a method for generating a transgenic cell. The method comprises transforming the cell with the nucleic acid construct provided herein. In another aspect, provided is a plant cell comprising the nucleic acid construct provided herein. In one embodiment, the nucleic acid construct is stably transformed into the plant cell. In another aspect, provided is a transgenic plant comprising the nucleic acid construct provided herein. In another aspect, provided is a method for expressing multiple genes in plant cells and/or tissues, comprising introducing into the plant cells and/or tissues the nucleic acid construct provided herein.

In one aspect, provided is a synthetic polynucleotide comprising a minimal core promoter element from an Ubiquitin-1 gene of Zea mays or Zea luxurians. In one embodiment, the minimal core promoter element comprises a polynucleotide sequence that is at least 65%, 70%, 75%, 80%, 85%, 90%, 95%, or 100% identical to SEQ ID NO: I or its complement. In a further or alternative embodiment, the minimal core promoter element comprises a polynucleotide sequence selected from the group consisting of SEQ NOs: 1 and 15-39. In a further embodiment, the minimal core promoter element comprising SEQ ID NO: 1 or its complement. In a further embodiment, the minimal core promoter element consists essentially of SEQ ID NO: 1 or its complement. In another embodiment, the synthetic polynucleotide further comprises an exon from an Ubiquitin-1 gene and an intron from an Ubiquitin-1 gene. In a further embodiment, the exon or intron is from an Ubiquitin-1 gene of Zea mays or Zea luxurians.

In another embodiment, the synthetic polynucleotide further comprises an upstream regulatory sequence from an Ubiquitin-1 gene. In a further embodiment, wherein the upstream regulatory sequence comprises a polynucleotide sequence that is at least 65%, 70%, 75%, 80%, 85%, 90%, 95%, or 100% identical to SEQ ID NO: 4 or its complement. In a further embodiment, wherein the upstream regulatory sequence comprises SEQ ID NO: 4 or its complement. In a further embodiment, wherein the upstream regulatory sequence consists essentially of SEQ ID NO: 4 or its complement.

In another embodiment, the synthetic polynucleotide further comprises at least one element selected from a list comprising an upstream regulatory sequence (URS), an enhancer element, an exon, an intron, a transcription start site, a TATA box, and a heat shock consensus element. In another embodiment, the synthetic polynucleotide further comprises a nucleotide sequence of interest operably linked to the minimal core promoter element. In another embodiment, the synthetic polynucleotide further comprises an element selected from the group consisting of an upstream regulatory sequence (URS), an enhancer element, an exon, an intron, a transcription start site, a TATA box, a heat shock consensus element, and combinations thereof. In another embodiment, the synthetic polynucleotide further comprises a nucleotide sequence of interest operably linked to the minimal core promoter element.

In another embodiment, the synthetic polynucleotide further comprises a second minimal core promoter element from Zea mays or Zea luxurians, wherein the two minimal core promoter elements are in reverse complimentary orientation with respect to each other in the polynucleotide. In a further or alternative embodiment, the synthetic polynucleotide further comprises an exon from an Ubiquitin-1 gene and an intron from an Ubiquitin-1 gene. In a further embodiment, the synthetic polynucleotide comprises a polynucleotide sequence that is at least 65%, 70%, 75%, 80%, 85%, 90%, 95%, or 100% identical to SEQ ID NO: 3 or its complement. In a further embodiment, the synthetic polynucleotide comprises SEQ ID NO: 3 or its complement. In a further embodiment, the synthetic polynucleotide consists essentially of SEQ ID NO: 3 or its complement.

In a further or alternative embodiment, the synthetic polynucleotide further comprises an upstream regulatory sequence from an Ubiquitin-1 gene. In a further embodiment, wherein the upstream regulatory sequence comprises a polynucleotide sequence that is at least 65%, 70%, 75%, 80%, 85%, 90%, 95%, or 100% identical to SEQ ID NO: 4 or its complement. In a further embodiment, the upstream regulatory sequence comprises SEQ ID NO: 4 or its complement. In a further embodiment, the upstream regulatory sequence consists essentially of SEQ ID NO: 4 or its complement.

In another embodiment, the synthetic polynucleotide comprising two minimal core promoter elements further comprises at least one element selected from a list comprising an upstream regulatory sequence (URS), an exon, an intron, a transcription start site, a TATA box, a heat shock consensus element, and a translational START and/or STOP nucleotide sequence. In a further or alternative embodiment, the synthetic polynucleotide comprising two minimal core promoter elements further comprises an element selected from the group consisting of an upstream regulatory sequence (URS), an exon, an intron, a transcription start site, a TATA box, a heat shock consensus element, a translational START and/or STOP nucleotide sequence, and combinations thereof. In a further embodiment, the synthetic polynucleotide comprises SEQ ID NO: 5 or its complement. In a further embodiment, the synthetic polynucleotide consists essentially of SEQ ID NO: 5 or its complement.

In another embodiment, the synthetic polynucleotide comprising two minimal core promoter elements comprises a first nucleotide sequence of interest operably linked to one of the minimal core promoter elements. In a further embodiment, the synthetic polynucleotide comprises a second nucleotide sequence of interest operably linked to the minimal core promoter element that is not operably linked to the first nucleotide sequence of interest.

In one embodiment of the synthetic polynucleotide provided, the exon is from an Ubiquitin-1 gene of a Zea spp. In one embodiment of the synthetic polynucleotide provided, the exon is from an Ubiquitin-1 gene of Zea mays or Zea luxurians. In another embodiment, the intron is from an Ubiquitin-1 gene of a Zea spp. In another embodiment, the intron is from an Ubiquitin-1 gene of Zea mays or Zea luxurians. In a further or alternative embodiment, the Zea spp. is Zea mays. In another embodiment, the Zea spp. is Zea luxurians.

In another aspect, provided is a method for producing a transgene cell. The methods comprise transforming the cell with the synthetic polynucleotide described herein. In one embodiment, the cell is a plant cell. In another aspect, provided is a plant cell comprising the synthetic polynucleotide described herein. In another aspect, provided is a plant comprising a plant cell comprising the synthetic polynucleotide described herein.

In another aspect, provided is a method for expressing a nucleotide sequence of interest in a plant cell. The method comprises introducing into the plant cell the nucleotide sequence of interest operably linked to a means for initiating transcription in a direction-independent manner. In another aspect, provided is a method for expressing a nucleotide sequence of interest in a plant cell. The method comprises introducing into the plant cell the nucleotide sequence of interest operably linked to a means for initiating transcription of two operably linked nucleotide sequences of interest. In a further embodiment, the method comprising introducing into the plant cell a nucleic acid comprising: (a) the nucleotide sequence of interest operably linked to the means for initiating transcription of two operably linked nucleotide sequences of interest; and (b) a second nucleotide sequence of interest operably linked to the means for initiating transcription of two operably linked nucleotide sequences of interest.

In a further or alternative embodiment, the means for initiating transcription of two operably linked nucleotide sequences of interest comprises SEQ ID NO: 5 or its complement. In a further or alternative embodiment, the means for initiating transcription of two operably linked nucleotide sequences of interest comprises SEQ ID NO: 5. In another embodiment, the means for initiating transcription of two operably linked nucleotide sequences of interest comprises complement of SEQ ID NO: 5. In another embodiment, the nucleic acid is introduced into the plant cell so as to target to a predetermined site in the DNA of the plant cell the nucleotide sequence of interest operably linked to the means for initiating transcription of two operably linked nucleotide sequences of interest. In a further or alternative embodiment, the nucleotide sequence of interest operably linked to the means for initiating transcription of two operably linked nucleotide sequences of interest is targeted to the predetermined site utilizing Zinc finger nuclease-mediated recombination.

In another aspect, provided is a nucleic acid construct for expressing multiple genes in plant cells and/or tissues. The nucleic acid construct comprises (a) a bi-directional promoter; and (b) two gene expression cassettes on opposite ends of the bi-directional promoter; wherein at least one of the gene expression cassettes comprises two or more genes linked via a translation switch.

In one embodiment, the nucleic acid construct does not comprise a viral sequence. In another embodiment, the bi-directional promoter does not comprise a viral sequence. In another embodiment, the bi-directional promoter comprises at least one enhancer. In another embodiment, the bi-directional promoter does not comprise an enhancer. In another embodiment, the nucleic acid construct comprises a binary vector for Agrobacterium-mediated transformation.

In one embodiment, the bi-directional promoter comprises an element selected from the group consisting of a cis-element or upstream regulatory sequence (URS), an enhancer element, an exon, an intron, a transcription start site, a TATA box, a heat shock consensus element, and combinations thereof. In a further or alternative embodiment, the bi-directional promoter comprises an upstream regulatory sequence (URS) from an Ubiquitin gene. In a further embodiment, the bi-directional promoter comprises (a) a promoter different from a promoter of an Ubiquitin gene and (ii) an upstream regulatory sequence (URS) from an Ubiquitin gene.

In another embodiment, the bi-directional promoter comprises a minimal core promoter element from an Ubiquitin-1 gene of Zea mays or Zea luxurians. In another embodiment, the bi-directional promoter further comprises a second minimal core promoter from Zea mays or Zea luxurians, wherein the two minimal core promoter elements are in reverse complimentary orientation with respect to each other. In a further embodiment, the minimal core promoter element comprises a polynucleotide sequence at least 65%, 70%, 75%, 80%, 85%, 90%, 95%, or 100% identical to SEQ ID NO:1 or its complement. In a further or alternative embodiment, the minimal core promoter element comprises a polynucleotide sequence selected from the group consisting of SEQ ID NOs: 1 and 15-39. In a further embodiment, the minimal core promoter element comprises a polynucleotide sequence selected from the group consisting of SEQ ID NOs: 1 and 15-34. In a further embodiment, the minimal core promoter element comprises a polynucleotide sequence selected from the group consisting of SEQ ID NOs: 1 and 15-29. In a further embodiment, the minimal core promoter element comprises a polynucleotide sequence selected from the group consisting of SEQ ID NOs: 1 and 15-24. In a further embodiment, the minimal core promoter element comprises a polynucleotide sequence selected from the group consisting of SEQ ID NOs: 1 and 15-19. In a further embodiment, the minimal core promoter element comprises a polynucleotide sequence of SEQ ID NO: 1.

In a further or alternative embodiment, the bi-directional promoter comprises an exon from an Ubiquitin-1 gene and/or an intron from an Ubiquitin gene. In a further embodiment, the bi-directional promoter comprises a polynucleotide of at least 75%, 80%, 85%, 90%, 95% or 100% identical to SEQ ID NO: 3 or its complement. In a further embodiment, the bi-directional promoter comprises a polynucleotide of SEQ ID NO: 3 or its complement. In another embodiment, the bi-directional promoter comprises an intron from an alcohol dehydrogenase gene. In one embodiment, the nucleic acid construct is stably transformed into transgenic plants. In one embodiment, the plants are monocotyledons plants. In another embodiment, the plants are dicotyledons plants. In another embodiment, the plants are not monocotyledons plants. In another embodiment, the plants are not dicotyledons plants.

In a further or alternative embodiment, the bi-directional promoter comprises an upstream regulatory sequence from an Ubiquitin gene. In a further embodiment, the upstream regulatory sequence from an Ubiquitin gene comprises a polynucleotide of sequence at least 75%, 80%, 85%, 90%, 95%, or 100% identical to SEQ ID NO: 4 or its complement. In a further embodiment, the upstream regulatory sequence from an Ubiquitin gene comprises a polynucleotide of SEQ ID NO: 4 or its complement. In another embodiment, the bi-directional promoter comprises a polynucleotide of at least 75%, 80%, 85%, 90%, 95%, or 100% identical to SEQ ID NO: 5 or its complement. In another embodiment, the bi-directional promoter comprises a polynucleotide of SEQ ID NO: 5 or its complement.

In one embodiment, both the gene expression cassettes comprise two or more genes linked via a translation switch. In a further or alternative embodiment, the translation switch is selected from the group consisting of an internal ribosome entry site (RES), an alternative splicing site, a ribozyme cleavage site, a polynucleotide sequence coding a 2A peptide, a polynucleotide sequence coding a 2A-like peptide, a polynucleotide sequence coding an intein, polynucleotide sequence coding a protease cleavage site, and combinations thereof. In a further or alternative embodiment, the translation switch comprises a cis-acting hydrolase element (CHYSEL), In a further embodiment, the CHYSEL is a 2A or 2A-like peptide sequence. In another embodiment, a gene upstream of the translational switch does not comprise a translation stop codon. In another embodiment, the nucleic acid construct enables or allows expression of at least four genes. In a further embodiment, all four genes are transgenes. In another embodiment, the nucleic acid construct enables expression of genes between three and twenty. In another embodiment, the nucleic acid construct enables expression of genes between four and eight. In a further or alternative embodiment, the genes are transgenes. In another embodiment, at least one gene expression cassette comprises a polynucleotide sequence encoding a fusion protein. In a further embodiment, the fusion protein comprises three to five genes.

In some embodiments, expression of genes from the bi-directional promoter is at least four-fold higher as compared to a uni-directional promoter. In some embodiments, expression of genes from the bi-directional promoter is from three to ten folds higher as compared to a uni-directional promoter. In some embodiments, expression of genes from the bi-directional promoter is from four to eight folds higher as compared to a uni-directional promoter. In some embodiments, a selection marker gene is placed at far end from the promoter (i.e., at the 3′ end of a gene expression cassette downstream of another gene).

In another aspect, provided is a method for generating a transgenic plant, comprising transforming a plant cell with the nucleic acid construct provided herein. In another aspect, provided is a method for generating a transgenic cell, comprising transforming the cell with the nucleic acid construct provided herein. In another aspect, provided is a plant cell comprising the nucleic acid construct provided herein. In a further or alternative embodiment, the nucleic acid construct is stably transformed into the plant cell. In another aspect, provided is a transgenic plant comprising the nucleic acid construct provided herein. In a further or alternative embodiment, the nucleic acid construct is stably transformed into cells of the transgenic plant. In another aspect, provide is a method for expressing multiple genes in plant cells and/or tissues, comprising introducing into the plant cells and/or tissues the nucleic acid construct provided herein. In a further or alternative embodiment, the plant cells and/or tissues are stably transformed with the nucleic acid construct provided herein. In another aspect, provided is a binary vector for Agrobacterium-mediated transformation. In one embodiment, the binary vector comprises the nucleic acid construct provided herein. In another embodiment, the binary vector comprises the synthetic polynucleotide provided herein. In another aspect, provided is the use of the bi-directional promoter provided herein for multiple-transgenes expression in plants.

### BRIEF DESCRIPTION OF THE DRAWINGS AND SEQUENCES

FIG. 1 shows an exemplary (not to scale) maize Ubi1 (ZmUbi1) promoter, which comprises an approximately 900 bp Upstream Element located 5′ of the transcription start site (TSS). The upstream element contains a TATA box (located approximately −30 bp of the TSS), and two overlapping heat shock consensus elements (located approximately −200 bp of the TSS). This promoter also comprises about 1100 bp 3′ of the TSS region. This 3′ region contains an adjacent leader sequence (ZmUbi1 exon), and an intron.

FIG. 2 shows an exemplary embodiment of the synthetic Ubi1 bidirectional promoter provided, which includes a minUbi1P minimal core element cloned upstream (in the reverse complementary orientation) of a ZmUbi1 promoter.

FIG. 3 shows an exemplary schematic drawing of yfp and GUS gene expression cassettes, which are each operably linked to a synthetic Ubi1 bidirectional promoter.

FIG. 4 shows a representative plasmid map of pDAB105801. FIG. 5 shows a representative plasmid map of pDAB108706. FIG. 6 shows a representative plasmid map of pDAB101556.

FIG. 7A shows SEQ ID NO: 1, which comprises a 215 bp region of a Zea mays Ubi1 minimal core promoter (minUbi1P). FIG. 7B shows SEQ ID NO:2, which comprises a Z. mays Ubi1 intron.

FIG. 8A shows SEQ ID NO: 3, which comprises the reverse complement of a polynucleotide comprising a Z. mays minUbi1P minimal core promoter (underlined); a Z. mays Ubi1 leader (ZmUbi1 exon; bold font); and a Z. mays Ubi1 intron (lower case). FIG. 8B shows SEQ ID NO: 4, which comprises a segment of a Z. mays Ubi1 upstream element, where element (and/or its reverse complement) may be located in a synthetic Ubi1 promoter with a minUbi1P element adjacent to its 5′ or 3′ end.

FIG. 9 shows SEQ ID NO: 5, which comprises an exemplary synthetic Ubi1 bidirectional promoter, wherein the reverse complement of a first minUbi1P, and a second minUbi1P, are underlined. FIG. 10 shows SEQ ID NO: 6, which comprises an exemplary nucleic acid comprising yfP and GUS gene expression cassettes driven by a synthetic Ubi1 bidirectional promoter.

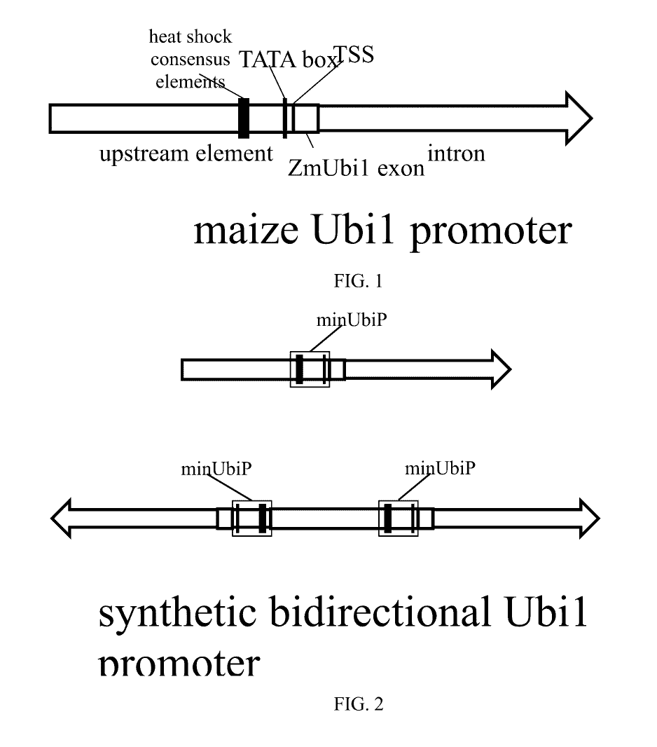

SEQ ID NO: 7 comprises a YFP Forward Primer: 5′- GATGCCTCAG TGGGAAAGG-3′. SEQ ID NO: 8 comprises a YFP Reverse Primer: 5′- CCATAGGTGA GAGTGGTGAC AA-3′. SEQ ID NO: 9 comprises an Invertase Forward Primer: 5′-TGGCGGACGA CGACTTGT-3′. SEQ ID NO: 10 comprises an Invertase Reverse Primer: 5′-AAAGTTTGGA GGCTGCCGT-3′. SEQ ID NO: 11 comprises an Invertase Probe: 5′-CGAGCAGACC GCCGTGTACT TCTACC-3′. SEQ ID NO: 12 comprises an AAD1 Forward Primer: 5′-TGTTCGGTFC CCTCTACCAA-3′. SEQ ID NO: 13 comprises an AAD1 Reverse Primer: 5′-CAACATCCAT CACCTTGACTGA-3′ SEQ ID NO: 14 comprises an AAD1 Probe: 5″-CACAGAACCG TCGCTTCAGC AACA-3′ (see also Table 7).

FIG. 11 shows a representative Western blot analysis confirming stable YFP and GUS expression driven by a bidirectional Z. mays Ubiquitin1 :Promoter construct (pDAB108706) in maize T₀ plants. Representative plants showed stable YFP expression in leaf driven by the Min-UbiP1 minimal core promoter element. The amount of protein which was produced is indicated as parts per million (ppm).

FIG. 12 shows a representative Western blot analysis showing stable YFP and GUS expression from the control construct containing a ZmUbi1 promoter that only drives expression of N′TP (pDAB101556); a GUS coding sequence is not contained in this construct. The amount of protein which was produced is indicated as parts per million (ppm).

FIG. 13A shows exemplary constructs of four-gene cassette stacks pDAB105843 [showing two cassettes of AAD1-2A-YFP (or Phiyfp) plus Cry34-2A-Cry35] and pDAB105846 [showing two cassettes of YFP (or Phiyfp)-2A-AND1 plus Cry34-2A-Cry35]. Shaded arrows indicate direction of transcription from the bi-directional promoter. Ubi1-minP comprises 200 nt sequence upstream of transcriptional start site of maize Ubi1 promoter. Ubi1-URS comprises maize Ubi1 promoter upstream regulatory region consisting of sequence upstream of transcription start site excluding the 200 nt minimal promoter (shown as arrow). Ubi1-Int comprises an intron of maize Ubi1 promoter. FIG. 13B shows additional exemplary binary constructs of four-gene cassette stacks from pDAB108717 and pDAB108718.

FIG. 14 shows exemplary schematic presentations of multi-gene constructs provided herein. Translation switches are shown using a special symbol.

FIG. 15 shows representative maps for plasmids pDAB105818 and pDAB105748.

FIG. 16 shows representative maps of plasmids pDAB105803 and pDAB105840.

FIG. 17 shows representative maps for plasmids pDAB105841 and pDAB105842.

FIG. 18 shows representative maps of plasmids pDA13105843 and pDA13101917.

FIG. 19 shows a representative map of plasmid pDAB108717.

FIG. 20 shows representative maps for plasmids pDA13105844 and pDAB105845.

FIG. 21 shows representative maps of plasmids pDAB105846 and pDAB108718.

FIG. 22A shows exemplary protein expression data for Cry35 of pDAB108717 and

FIG. 22B pDAB108718. FIG. 23 shows nucleic acid sequence for gene expression cassettes of pDAB108717, where each gene and element is illustrated.

FIGS. 24A-E shows additional minimal core promoters (min-Ubi1P or Ubi1-minP) of SEQ NOs: 15-39.

FIG. 25 shows two exemplary sequences for yellow fluorescent proteins from Phialidium sp. SL-2003 (Phiyfp, SEQ ID NO: 50; and Phiyfpv3, SEQ ID NO: 51).

FIG. 26 shows exemplary embodiments of the synthetic Ubi1 bidirectional promoter and constructs provided, including pDAB108706 (ZMUbi1 bidirectional (−200)) and pDAB108707 (ZMUbi bidirectional (−90)). pDAB101556 (ZmUbi1-YFP control) and pDAB108716 (ZMUbi1 without minimal promoter) serve as control constructs with uni-directional promoters.

FIG. 27A shows exemplary expression results (V6) from the four constructs shown in FIG. 26 for YFP protein (LCMS) in ng/cm2. FIG. 27B shows exemplary relative expression results (V6) from the four constructs shown in FIG. 26 for YFP RNA.

FIG. 28A shows exemplary expression results (V6) from the four constructs shown in FIG. 26 for GUS protein (LCMS) in ng/cm2. FIG. 28B shows exemplary relative expression results (V6) from the four constructs shown in FIG. 26 for GUS RNA.

FIG. 29A shows exemplary expression results (V6) from the four constructs shown in FIG. 26 for AAD1 protein (LCMS) in ng/cm2, FIG. 2913 shows exemplary relative expression results (V6) from the four constructs shown in FIG. 26 for AAD1 RNA.

FIG. 30A shows a statistical analysis of expression results (V6) from the four constructs shown in FIG. 26 for YFP protein (LCMS) in ng/cm2. The mean values for pDAB108707, pDAB108706, pDAB101556, and pDAB108716 are 57.63, 52.66, 49.75, and 0 respectively. FIG. 30B shows a statistical analysis of relative expression results (V6) from the four constructs shown in FIG. 26 for YFP RNA. The mean values for pDAB108706, pDAB108707, pDAB101556, and pDAL3108716 are 9.96, 8.07, 6.95, and 1.01 respectively.

FIG. 31A shows a statistical analysis of expression results (V6) from the four constructs shown in FIG. 26 for GUS protein (LCMS) in ng/cm2. The mean values for pDAB108706, pDAB108707, pDAB101556, and pDAB108716 are 151.27, 143.22, 0, and 213.17 respectively. FIG. 31B shows a statistical analysis of relative expression results (V6) from the four constructs shown in FIG. 26 for GUS RNA. The mean values for pDAB108706, pDAB108707, pDAB101556, and pDAB108716 are 0.65, 0.78, 0, and 3.03 respectively.

FIG. 32A shows a statistical analysis of expression results (V6) from the four constructs shown in FIG. 26 for AAD1 protein (LCMS) in ng/cm2. The mean values for pDAB108706, pDAB108707, pDAB101556, and pDAB108716 are 710.88, 1417.01, 856.58, and 1795.43 respectively. FIG. 32B shows a statistical analysis of relative expression results (V6) from the four constructs shown in FIG. 26 for AND1 RNA. The mean values for pDAB108706, pDAB108707, pDAB101556, and pDAB108716 are 1.33, 1.37, 1.93, and 2.93 respectively.

FIGS. 33A, 33B, and 33C show exemplary expression results (V10) from the four constructs shown in FIG. 26 for YFPAAD1, and GUS protein (LCMS) in ng/cm2 respectively.

FIGS. 34A, 34B, and 34C show statistical analysis of expression results (V10) from the four constructs shown in FIG. 26 for YFP, GUS, and AAD1 protein (LEMS) in ng/cm2 respectively. The mean values for pDAB108706, pDAB108707, pDAB101556, and pDAB108716 for YFP (FIG. 34A) are 71.77, 81.81, 49.58, and 23.01 respectively. The mean values for pDAB108706, pDAB1.08707, pDAB101556, and pDAB108716 for GUS (FIG. 34B) are 109.63, 98.25, 0, and 138.02 respectively. The mean values for pDAB108706, pDAB108707, pDAB101556, and pDAB108716 for AAD1 (FIG. 34C) are 666.11, 597.80, 715,12, and 1002.84 respectively.

FIGS. 35A, 35B, and 35C show exemplary expression results (R3) from the four constructs shown in FIG. 26 for YFP, GUS, and AAD1 protein (LCMS) in ng/cm2 respectively.

FIGS. 36A, 36B, and 36C show statistical analysis of expression results (R3) from the four constructs shown in FIG. 26 for YFP, GUS, and AAD1 protein (LCMS) in ng/cm2 respectively. The mean values for pDAB108706, pDAB108707, pDAB101556, and pDAB108716 for YFP (FIG. 36A) are 91.38, 49.49, 21.67, and 0.40 respectively. The mean values for pDAB108706, pDAB108707, pDAB101556, and pDAB108716 for GUS (FIG. 36B) are 5.52, 16.81, 1.07, and 46,60 respectively. The mean values for pDAB108706, pDAB108707, pDAB101556, and pDAB108716 for AAD1 (FIG. 36C) are 156.71, 153.44, 165.40, and 197.80 respectively.

FIG. 37A shows exemplary relative expression results (V6) of Cry34 RNA from the four constructs pDAB105748 (ZMUbi1-YFP), pDAB105818 (ZMUbi1-Cry34/ZMUbi1-Cry35/ZMUbi1-AAD1), pDAB108717 (YFP/AAD-1-ZMUbi1 bidirectional-Cry34-Cry35), and pDAB108718 (AAD1/YFP-ZMUbi1 bidirectinal-Cry34-Cry35). FIG. 37B shows exemplary relative expression results (V6) of Cry34 protein (LCMS) from the same four constructs pDAB105748, pDAB105818, pDAB108717, and pDAB108718.

FIG. 38A shows exemplary relative expression results (V6) of AAD1. RNA from the four constructs pDAB105748, pDAB105818, pDAB108717and pDAB10871.8. FIG. 38B shows exemplary relative expression results (V6) of AAD1 protein (LCMS) from the same four constructs pDAB105748, pDAB105818, pDAB108717, and pDAB108718.

FIG. 39A shows exemplary relative expression results (V6) of YFP RNA from the four constructs pDAB105748, pDAB105818, pDAB108717, and pDAB108718. FIG. 39B shows exemplary relative expression results (V6) of YFP protein (LOVIS) from the same four constructs pDAB105748, pDAB105818, pDAB108717, and pDAB108718.

FIG. 40A shows exemplary relative expression results (V6) of Cry35 RNA from the four constructs pDAB105748, pDAB105818, pDAB108717, and pDAB108718. FIG. 40B shows exemplary relative expression results (V6) of Cry35 protein (ELISA) from the same four constructs pDAB105748, pDAB105818, pDAB108717, and pDAB108718.

FIG. 41 shows exemplary relative expression results (V6) of PAT RNA from the four constructs pDAB105748, pDAB105818, pDAB108717, and pDAB108718.

FIG. 42A shows a statistical analysis of expression results (V6) of Cry 34 RNA from the four constructs pDAB105748, pDAB105818, pDAB108717, and pDAB108718 with the mean values 0, 2.42, 2.67, and 2.25 respectively. FIG. 42B shows a statistical analysis of expression results (V6) of Cry-34 protein from the same four constructs pDAB105748, pDAB105818, pDAB108717, and pDAB108718 with the mean values 0, 596.94, 2044.73, and 719.18 respectively.

FIG. 43A shows a statistical analysis of expression results (V6) of AAD1 RNA from the four constructs pDAB105748, pDAB105818, pDAB108717, and pDAB10871.8 with the mean values 0, 1.98, 2.68, and 2.03 respectively. FIG. 43B shows a statistical analysis of expression results (V6) of AAD1 protein from the same four constructs pDAB105748, pDAB105818, pDAB108717, and pDA13108718 with the mean values 0, 2237.54, 5763.88, and 2379.15 respectively.

FIG. 44A shows a statistical analysis of expression results (V6) of YFP RNA from the four constructs pDAB105748, pDAB105818, pDAB108717, and pDAB108718 with the mean values 3.59, 0, 2,78, and 1.95 respectively. FIG. 44B shows a statistical analysis of expression results (V6) of YFP protein from the same four constructs pDAB105748, pDAB105818, pDAB108717, and pDAB108718 with the mean values 1420.69, 251.68, 1154.04, and 706.04 respectively.

FIG. 45A shows a statistical analysis of expression results (V6) of Cry35 RNA from the four constructs pDAB105748, pDA13105818, pDAB108717, and pDAB108718 with the mean values 0, 1.12, 3.74, and 3.20 respectively. FIG. 45B shows a statistical analysis of expression results (V6) of Cry35 protein from the same four constructs pDAB105748, pDAB105818, pDAB108717, and pDAB108718 with the mean values 0, 283.54, 635.83, and 90.97 respectively.

FIG. 46 shows a statistical analysis of expression results (V6) of PAT RNA from the four constructs pDAB105748, pDAB105818, pDAB108717, and pDAB108718 with mean values 1,56, 0.07, 1.46, and 1.01 respectively.

FIGS. 47A, 47B, 47C, and 47D show exemplary protein expression results (V10) of YFP, AAD1, Cry34, and Cry35 respectively from the four constructs pDAB105748, pDAB105818, pDAB108717, and DAB108718.

FIGS. 48A, 48B, 48C, and 48D show statistical analysis of protein expression results (V10) of YFP, AAD1, Cry-34, and Cry35 respectively from the four constructs pDAB105748, pDAB105818, pDAB108717, and pDAB108718. The mean values for YFP (FIG. 48A) are 1033.47, 27.51, 136.18, and 119.06 respectively. The mean values for AAD1 (FIG. 48B) are 80.89, 1323.80, 1544.69, and 802.50 respectively. The mean values for Cry34 (FIG. 48C) are 0.246.05, 1089,18, and 769.81 respectively. The mean values for Cry35 (FIG. 48D) are 0, 90.75, 106.09, and 88.80 respectively.

FIGS. 49A, 49B, 49C, and 49D show exemplary protein expression results (R3) of YFP, AAD1, Cry34, and Cry35 respectively from the four constructs pDAB105748, pDAB105818, pDAB108717, and pDAB108718.

FIGS. 50A, 50B, 50C, and 50D show statistical analysis of protein expression results (R3) of YFP, AAD1, Cry34, and Cry35 respectively from the four constructs pDAB105748, pDAB105818, pDAB108717, and pDAB108718. The mean values for YFP (FIG. 50A) are 2589.63, 43.62, 1305.27, and 1727.96 respectively. The mean values for AAD1 (FIG. 50B) are 244.41, 1803.99, 1642.44, and 1279.17 respectively. The mean values for Cry34 (FIG. 50C) are 422.45, 7258.15, 9285.74, and 7544.75 respectively. The mean values for Cry35 (FIG. 50D) are 0, 373.35, 441.11, and 348.45 respectively.

FIG. 51 shows exemplary results of Western blot for protein expression of Cry34, Cry35, and AAD1 from pDAB108718 and pDAB108717.

### DETAILED DESCRIPTION OF THE INVENTION

Development of transgenic products is becoming increasingly complex, which requires pyramiding multiple transgenes into a single locus. Traditionally each transgene usually requires a unique promoter for expression, so multiple promoters are required to express different transgenes within one gene stack. In addition to increasing the size of the gene stack, this frequently leads to repeated use of the same promoter to obtain similar levels of expression patterns of different transgenes controlling the same trait. Multi-gene constructs driven by the same promoter are known to cause gene silencing, thus making transgenic products less efficacious in the field. Excess of transcription factor (TF)-binding sites due to promoter repetition can cause depletion of endogenous TFs leading to transcriptional inactivation.

Provided are constructs and methods combining the bidirectional promoter system with bicistronic organization of genes on either one or both ends of the promoter, for example with the use of a 2A sequence from Thosea asigna virus. The 2A protein, which is only 16-20 amino acids long, cleaves the polyprotein at its own carboxyl-terminus. This “self-cleavage” or “ribosome skip” property of the 2A or 2A-like peptide can be used to process artificial polyproteins produced in transgenic plants. In one embodiment, Cry34 and Cry35 genes are fused in one gene expression cassette, where YFP (or Phiyfp) and AAD1 genes are fused into another gene expression cassette (with a single open reading frame (ORF) with a copy of the 2A protein gene placed between the two genes in each combination). For example, each of these gene expression cassettes (or gene pairs) can be placed on the either end of the bidirectional promoter to drive 4 transgenes using a single promoter. Thus, the constructs and methods provided herein are useful to avoid repeated use of the same promoter and significantly reduce the size of commercial constructs. In addition, driving four or more genes with one promoter also provides ability to co-express genes controlling a single trait.

Plant promoters used for basic research or biotechnological application are generally unidirectional, directing only one gene that has been fused at its 3′ end (downstream). It is often necessary to introduce multiple genes into plants for metabolic engineering and trait stacking and therefore, multiple promoters are typically required in future transgenic crops to drive the expression of multiple genes. It is desirable to design strategies that can save the number of promoters deployed and allow simultaneous co-regulated expression for gene pyramiding. In some embodiment, the bi-directional promoters provided can drive transcription of multiple transcription units, including RNAi, artificial miRNA, or hairpin-loop RNA sequences.

One approach for reducing the number of promoters deployed is the use of critical transcription activating switches that may drive transcription in both directions. These promoters are called bi-directional promoters. Synthetic promoters can be designed to limit the level of homology among multiple promoters to be used for genetic engineering in crop plants, which may avoid homology based gene silencing. Artificially designed bi-directional promoters can be valuable tools for the development of transgenic plants. Bi-directional function of promoters in plants has been reported in some cases, including the CaMV 35S and the mannopine synthase promoter (mas) promoters. However, suitability of using such promoters has not been examined for predictable, stable and simultaneous expression of genes in the two directions.

Another method for coordinate expression of multiple genes is to encode a single open reading frame into a polyprotein precursor containing short intervening motif with self processing properties between two coding sequences. Autocatalytic processing of the polyprotein precursor leads to release of multiple independent proteins resulting into their synchronized coordinated expression. A synthetic self-hydrolyzing 2A peptide sequence has been used both in plant and animal system to express two transgenes. The 2A peptide sequence is utilized by several known viruses and consists of 16-20 amino acids. This 2A peptide sequence self-cleaves (or ribosome skip) co-translationally by modifying the activity of the ribosome to allow hydrolysis of the 2A between two proteins resulting in the release of the two protein products.

Provided are constructs and methods combining bi-directional promoter approach with polyprotein processing using intervening synthetic motifs, where expression of at least 4 transgenes using a single promoter can be readily achieved. Genes of Cry34 and Cry35, and genes of YFP (or Phiyfp) and AAD1 have been fused as gene expression cassettes or gene pairs into single open reading frames (ORF) with a copy of the 2A protein gene placed between the genes. The gene pairs can be placed on either end of the bidirectional promoter to drive four transgenes using one single promoter. The constructs and/or methods provided herein are useful to avoid repeated use of the same promoter avoiding potential transgene silencing problems. In addition, this transgene design approach can significantly reduce the size of the transgene stacks containing multiple transgenes. Driving four or more genes with one promoter also provides ability to co-express genes controlling a single trait ensuring long-term efficacy of transgenic products.

Development of transgenic plants is becoming increasingly complex, and typically requires stacking multiple transgenes into a single locus. See Xie et al. (2001) Nat. Biotechnol. 19(7):677-9, Since each transgene usually requires a unique promoter for expression, multiple promoters are required to express different transgenes within one gene stack. In addition to increasing the size of the gene stack, this frequently leads to repeated use of the same promoter to obtain similar levels of expression patterns of different transgenes. This approach is often problematic, as the expression of multiple genes driven by the same promoter may lead to gene silencing or HBGS. An excess of competing transcription factor (TF)-binding sites in repeated promoters may cause depletion of endogenous TFs and lead to transcriptional down regulation. The silencing of transgenes will likely undesirably affect the performance of a transgenic plant produced to express the transgenes. Repetitive sequences within a transgene may lead to gene intra-locus homologous recombination resulting in polynucleotide rearrangements.

Plant promoters used for basic research or biotechnological application are generally unidirectional, and regulate only one gene that has been fused at its 3′ end (downstream). To produce transgenic plants with various desired traits or characteristics, it would be useful to reduce the number of promoters that are deployed to drive expression of the transgenes that encode the desired traits and characteristics. It is often necessary to introduce multiple transgenes into plants for metabolic engineering and trait stacking, thereby necessitating multiple promoters to drive the expression of multiple transgenes. By developing a single synthetic bidirectional promoter that can drive expression of two transgenes that flank the promoter, the total numbers of promoters needed for the development of transgenic crops may be reduced, thereby lessening the repeated use of the same promoter, reducing the size of transgenic constructs, and/or reducing the possibility of HBGS.

Embodiments herein utilize a process wherein a unidirectional promoter from a maize ubiquitin-1 gene (e.g., ZmUbi1) is used to design a synthetic bidirectional promoter, such that one promoter can direct the expression of two genes, one on each end of the promoter. Processes as utilized herein may comprise identification of the Ubi1 minimal core promoter element (minUbi1P) from a ZmUbi1 gene, and engineering of this element into new contexts to construct certain synthetic bidirectional promoters. Synthetic bidirectional promoters, such as may be created by a process according to some embodiments of the invention, may allow those in the art to stack transgenes in plant cells and plants while lessening the repeated use of the same promoter and reducing the size of transgenic constructs. Furthermore, regulating the expression of two genes with a single synthetic bidirectional promoter may also provide the ability to co-express the two genes under the same conditions, such as may be useful, for example, when the two genes each contribute to a single trait in the host. The use of bidirectional promoters in plants has been reported in some cases, including the CAMV 35 promoters (Barfield and Pua (1991) Plant Cell Rep. 10(6-7):308-14; Xie et al. (2001), and the mannopine synthase promoter (mas) promoters (Velten et al, (1984) EMBO J. 3(12):2723-30; Langridge et al. (1989) Proc. Natl. Acad. Sci. USA 86:3219-23).

Transcription initiation and modulation of gene expression in plant genes is directed by a variety of DNA sequence elements that are collectively arranged within the promoter. Eukaryotic promoters consist of minimal core promoter element (minP), and further upstream regulatory sequences (URSs). The core promoter element is a minimal stretch of contiguous DNA sequence that is sufficient to direct accurate initiation of transcription. Core promoters in plants also comprise canonical regions associated with the initiation of transcription, such as CAAT and TATA boxes. The TATA box element is usually located approximately 20 to 35 nucleotides upstream of the initiation site of transcription.

The activation of the minP is dependent upon the URS, to which various proteins bind and subsequently interact with the transcription initiation complex. URSs comprise DNA sequences that determine the spatiotemporal expression pattern of a promoter comprising the URS. The polarity of a promoter is often determined by the orientation of the minP, while the LYRS is bipolar (i.e., it functions independent of its orientation). For example, the CaMV 35S synthetic unidirectional polar promoter may be converted to a bidirectional promoter by fusing a minP at the 5′ end of the promoter in the opposite orientation. See, for example, Xie et al. (2001) Nat. Biotechnol, 19(7):677-9.

Certain abbreviations disclosed are listed in Table 1.

TABLE 1
Abbreviations used in the disclosure

| Phrase | Abbreviation |
|---|---|
| bicinchoninic acid | BCA |
| cauliflower mosaic virus | CaMV |
| chloroplast transit peptide | CTP |
| homology-based gene silencing | HBGS |
| ZmUbi1 minimal core promoter | minUbi1P |
| oligo ligation amplification | OLA |
| phosphate buffered saline | PBS |
| phosphate buffered saline with 0.05% Tween 20 | PBST |
| polymerase chain reaction | PCR |
| rolling circle amplification | RCA |
| reverse transcriptase PCR | RT-PCR |
| single nucleotide primer extension | SNuPE |
| upstream regulatory sequence | URS |
| Zea mays Ubiquitin-1 gene | ZmUbi1 |

In specific examples of some embodiments, modified elements of a maize Ubi1 (ZmUbi1) promoter derived from the Z. mays inbred line, B73, are used to engineer synthetic bidirectional promoters that may function in plants to provide expression control characteristics that are unique with respect to previously available bidirectional promoters. This ZmUbi1 promoter originally derived from B73 comprises sequences located in the maize genome within about 899 bases 5′ of the transcription start site, and further within about 1093 bases 3′ of the transcription start site. Christensen et al. (1992) Plant Mol. Biol. 18(4):675-89 (describing a B73 ZmUbi1 gene). A modified ZmUbi1 promoter derived from B73 that is used in some examples is an approximately 2 kb promoter that contains a TATA box; two overlapping heat shock consensus elements; an 82 or 83 nucleotide (depending on the reference strand) leader sequence immediately adjacent to the transcription start site, which is referred to herein as ZmUbi1 exon; and a 1015-1016 nucleotide intron (see FIG. 1 for example). Other maize ubiquitin promoter variants derived from Zea species and Zea mays genotypes may exhibit high sequence conservation around the minP element consisting of the TATA element and the upstream heat shock consensus elements. Thus, embodiments of the invention are exemplified by the use of this short (−200 nt) highly-conserved region (e.g., SEQ ID NO: 1) of a ZmUbi1 promoter as a minimal core promoter element for constructing synthetic bidirectional plant promoters.

As used herein, the articles, “a,” “an,” and “the” include plural references unless the context clearly and unambiguously dictates otherwise.

As used herein, the phrase “backcrossing” refers to a process in which a breeder crosses hybrid progeny back to one of the parents, for example, a first generation hybrid F1 with one of the parental genotypes of the F1 hybrid.

As used herein, the phrase “intron” refers to any nucleic acid sequence comprised in a gene (or expressed nucleotide sequence of interest) that is transcribed but not translated. Intron is different from 5′ end untranslated leader sequence which is not considered as part of a gene. Introns include untranslated nucleic acid sequence within an expressed sequence of DNA, as well as the corresponding sequence in RNA molecules transcribed therefrom.

As used herein, the phrase “isolated” refers to biological component (including a nucleic acid or protein) has been substantially separated, produced apart from, or purified away from other biological components in the cell of the organism in which the component naturally occurs (i.e., other chromosomal and extra-chromosomal DNA and RNA, and proteins) while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods. The phrase “isolated” also embraces nucleic acids and proteins prepared by recombinant expression in a host cell, as well as chemically-synthesized nucleic acid molecules, proteins, and peptides.

As used herein, the phrase “gene expression” refers to a process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation. Northern blot, RT-PCR, Western blot, or in vitro, in situ, or in vivo protein activity assay(s).

As used herein, the phrase “homology-based gene silencing” (HBGS) refers to a generic term that includes both transcriptional gene silencing and posttranscriptional gene silencing. Silencing of a target locus by an unlinked silencing locus can result from transcription inhibition (transcriptional gene silencing; TGS) or mRNA degradation (post-transcriptional gene silencing; PTGS ), owing to the production of double-stranded RNA (dsRNA) corresponding to promoter or transcribed sequences, respectively. The involvement of distinct cellular components in each process suggests that dsRNA-induced TGS and PTGS likely result from the diversification of an ancient common mechanism. However, a strict comparison of TGS and PTGS has been difficult to achieve because it generally relies on the analysis of distinct silencing loci. A single transgene locus can be described to trigger both TGs and PTGS, owing to the production of dsRNA corresponding to promoter and transcribed sequences of different target genes. See, for example, Mourrain et al. (2007) Planta 225:365-79. It is likely that siRNAs are the actual molecules that trigger TGS and PTGS on homologous sequences: the siRNAs would in this model trigger silencing and methylation of homologous sequences in cis and in trans through the spreading of methylation of transgene sequences into the endogenous promoter.

As used herein, the phrase “nucleic acid molecule” (or “nucleic acid” or “polynucleotide”) refers to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide.” A nucleic acid molecule is usually at least 10 bases in length, unless otherwise specified. The term may refer to a molecule of RNA or DNA of indeterminate length. The term includes single- and double-stranded forms of DNA. A nucleic acid molecule may include either or both naturally-occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internudeotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anotneric nucleic acids, etc.). The term “nucleic acid molecule” also includes any topological conformation, including single-stranded, double-stranded, partially duplexed, triplexed, hairpinned, circular, and padlocked conformations.

Transcription proceeds in a 5′ to 3′ manner along a DNA strand. This means that RNA is made by the sequential addition of ribonucleotide-5′-triphosphates to the 3′ terminus of the growing chain (with a requisite elimination of the pyrophosphate). In either a linear or circular nucleic acid molecule, discrete elements (e.g., particular nucleotide sequences) may be referred to as being “upstream” relative to a further element if they are bonded or would be bonded to the same nucleic acid in the 5′ direction from that element. Similarly, discrete elements may be “downstream” relative to a further element if they are or would be bonded to the same nucleic acid in the 3′ direction from that element.

As used herein, the phrase “base position,” refers to the location of a given base or nucleotide residue within a designated nucleic acid. The designated nucleic acid may be defined by alignment (see below) with a reference nucleic acid.

As used herein, the phrase “hybridization” refers to a process where oligonucleotides and their analogs hybridize by hydrogen bonding, which includes Watson-Crick, Hoogsteen or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

As used herein, the phrases “specifically hybridizable” and “specifically complementary” refers to a sufficient degree of complementarity such that stable and specific binding occurs between the oligonucleotide and the DNA or RNA target. The oligonucleotide need not be 100% complementary to its target sequence to be specifically hybridizable. An oligonucleotide is specifically hybridizable when binding of the oligonucleotide to the target DNA or RNA molecule interferes with the normal function of the target DNA or RNA, and there is sufficient degree of complementarity to avoid non-specific binding of the oligonucleotide to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the chosen hybridization method and the composition and length of the hybridizing nucleic acid sequences. Generally, the temperature of hybridization and the ionic strength (especially the Na+ and/or Mg2+ concentration) of the hybridization buffer will contribute to the stringency of hybridization, though wash times also influence stringency. Calculations regarding hybridization conditions required for attaining particular degrees of stringency are discussed in Sambrook et al. (ed.), Molecular Cloning: A Laboratory Manual, 2nd ed., vol. 1-3, Cold Spring Harbor Laboratory Press, Cold Spring Harbor, New York, 1989, chs, 9 and 11.

As used herein, the phrase “stringent conditions” encompass conditions under which hybridization will only occur if there is less than 50% mismatch between the hybridization molecule and the DNA target. “Stringent conditions” include further particular levels of stringency. Thus, as used herein, “moderate stringency” conditions are those under which molecules with more than 50% sequence mismatch will not hybridize; conditions of “high stringency” are those under which sequences with more than 20% mismatch will not hybridize; and conditions of “very high stringency” are those under which sequences with more than 10% mismatch will not hybridize.

In particular embodiments, stringent conditions can include hybridization at 65° C., followed by washes at 65° C. with 0.1×SSC/0.1% SDS for 40 minutes.

The following are representative, non-limiting hybridization conditions:
Very High Stringency: Hybridization in 5×SSC buffer at 65° C. for 16 hours; wash twice in 2×SSC buffer at room temperature for 15 minutes each; and wash twice in 0.5×SSC buffer at 65 for 20 minutes each.
High Stringency: Hybridization in 5×-6×SSC buffer at 65-70 for 16-20 hours; wash twice in 2×SSC buffer at room temperature for 5-20 minutes each; and wash twice in 1×SSC buffer at 55-70° C. for 30 minutes each.
Moderate Stringency: Hybridization in 6×SSC buffer at room temperature to 55° C. for 16-20 hours; wash at least twice in 2×-3×SSC buffer at room temperature to 55° C. for 20-30 minutes each.

In particular embodiments, specifically hybridizable nucleic acid molecules can remain bound under very high stringency hybridization conditions. In these and further embodiments, specifically hybridizable nucleic acid molecules can remain bound under high stringency hybridization conditions. In these and further embodiments, specifically hybridizable nucleic acid molecules can remain bound under moderate stringency hybridization conditions.

As used herein, the phrase “oligonucleotide” refers to a short nucleic acid polymer. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of small DNA sequences. In PCR, the oligonucleotide is typically referred to as a “primer,” which allows a DNA polymerase to extend the oligonucleotide and replicate the complementary strand.

As used herein, the phrase “sequence identity” or “identity,” refers to a context where two nucleic acid or polypeptide sequences, may refer to the residues in the two sequences that are the same when aligned for maximum correspondence over a specified comparison window.

As used herein, the phrase “percentage of sequence identity” refers to the value determined by comparing two optimally aligned sequences (e.g., nucleic acid sequences, and amino acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et at (1990) J. Mol. Biol. 215: 403-10,

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method.

As used herein, the phrase “operably linked” refers to a context where the first nucleic acid sequence is operably linked with a second nucleic acid sequence when the first nucleic acid sequence is in a functional relationship with the second nucleic acid sequence. For instance, a promoter is operably linked with a coding sequence when the promoter affects the transcription or expression of the coding sequence. When recombinantly produced, operably linked nucleic acid sequences are generally contiguous and, where necessary to join two protein-coding regions, in the same reading frame. However, elements need not be contiguous to be operably linked,

As used herein, the phrase “promoter” refers to a region of DNA that generally is located upstream (towards the 5′ region of a gene) that is needed for transcription. Promoters may permit the proper activation or repression of the gene which they control. A promoter may contain specific sequences that are recognized by transcription factors. These factors may bind to the promoter DNA sequences and result in the recruitment of RNA polymerase, an enzyme that synthesizes RNA from the coding region of the gene.

As used herein, the phrase “transforms” or “transduces” refers to a process where a virus or vector transfers nucleic acid molecules into a cell. A cell is “transformed” by a nucleic acid molecule “transduced” into the cell when the nucleic acid molecule becomes stably replicated by the cell, either by incorporation of the nucleic acid molecule into the cellular genome or by episomal replication. As used herein, the term “transformation” encompasses all techniques by which a nucleic acid molecule can be introduced into such a cell. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterim-mediated transfer (Fraley et al. (1983) Proc. Natl. Acad. Sci. USA 80:4803-7); direct DNA uptake; whiskers-mediated transformation; and microprojectile bombardment (Klein et al. (1987) Nature 327:70).

As used herein, the phrase “transgene” refers to an exogenous nucleic acid sequence. In one example, a transgene is a gene sequence (e.g., an herbicide-resistance gene), a gene encoding an industrially or pharmaceutically useful compound, or a gene encoding a desirable agricultural trait. In yet another example, the transgene is an antisense nucleic acid sequence, wherein expression of the antisense nucleic acid sequence inhibits expression of a target nucleic acid sequence. A transgene may contain regulatory sequences operably linked to the transgene (e.g., a promoter). In some embodiments, a nucleic acid sequence of interest is a transgene. However, in other embodiments, a nucleic acid sequence of interest is an endogenous nucleic acid sequence, wherein additional genomic copies of the endogenous nucleic acid sequence are desired, or a nucleic acid sequence that is in the anti sense orientation with respect to the sequence of a target nucleic acid molecule in the host organism.

As used herein, the phrase “vector” refers to a nucleic acid molecule as introduced into a cell, thereby producing a transformed cell. A vector may include nucleic acid sequences that permit it to replicate in the host cell, such as an origin of replication. Examples include, but are not limited to, a plasmid, cosmid, bacteriophage, or virus that carries exogenous DNA into a cell. A vector can also include one or more genes, antisense molecules, and/or selectable marker genes and other genetic elements known in the art. A vector may transduce, transform, or infect a cell, thereby causing the cell to express the nucleic acid molecules and/or proteins encoded by the vector. A vector may optionally include materials to aid in achieving entry of the nucleic acid molecule into the cell (e.g., a liposome).

As used herein, the phrase “plant” includes plants and plant parts including but not limited to plant cells and plant tissues such as leaves, stems, roots, flowers, pollen, and seeds. The class of plants that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicotyledons plants and monocotyledons plants. Examples of dicotyledons plants include tobacco, Arabidopsis, soybean, tomato, papaya, canola, sunflower, cotton, alfalfa, potato, grapevine, pigeon pea, pea. Brassica, chickpea, sugar beet, rapeseed, watermelon, melon, pepper, peanut, pumpkin, radish, spinach, squash, broccoli, cabbage, carrot, cauliflower, celery, Chinese cabbage, cucumber, eggplant, and lettuce. Examples of monocotyledons plants include corn, rice, wheat, sugarcane, barley, rye, sorghum, orchids, bamboo, banana, cattails, lilies, oat, onion, millet, and triticale.

As used herein, the phrase “plant material” refers to leaves, stems, roots, flowers or flower parts, fruits, pollen, egg cells, zygotes, seeds, cuttings, cell or tissue cultures, or any other part or product of a plant. In some embodiment, plant material includes cotyledon and leaf.

As used herein, the phrase “translation switch” refers to a mechanism at end of a gene allowing translation of an immediate downstream gene. The mechanism of translation switch can function at nucleic acid level (for example, viral or eukaryotic internal ribosome entry site (IRES), an alternative splicing site, or a ribozyme cleavage site or at peptide/protein leve or example, a 2A peptide, a 2A-like peptide, an intein peptide, or a protease cleavage site). Previously 2A and 2A-like peptides have been disclosed in US. Pat. No. 8,945,876, and the content of which is hereby incorporated by reference in its entirety. In addition, a linker sequence for multi-gene expression has been disclosed in US. Patent Application Publication Nos. 2007/0277263 and 2014/0259231, and the content of which are hereby incorporated by reference it their entireties.

These mechanisms of translation switch at nucleic acid level or at peptide/protein level are well known in the art. See e.g., Ii, Z., HM. Schumacher, et al. (2010) J Biotechnol 145(1): 9-16; Chen, Y., K. Perumal, et al. (2000) Gene Expr 9(3): 133-143; Dinkova, T. D., H. Zepeda, et al. (2005) Plant J 41(5): 722-731; Dorokhov, Y. L., M. V. Skulachev, et al. (2002) Proc Nati Acad Sci U S A 99(8): 5301-5306; Fernandez-Miragal I, O. and C. Hernandez (2011) PLoS One 6(7): e22617; Groppelli, E., G. J. Belsham, et al. (2007) J Gen Virol 88(Pt 5): 1583-1588; Ha, S. H., Y. S. Liang, et al. (2010) Plant Biotechnol J 8(8): 928-938; Karetnikov, A. and K. Lehto (2007) J Gen Virol 88(Pt 1): 286-297; Karetnikov, A. and K. Lehto (2008) Virology 371(2): 292-308; Khan, M. A., H. Yumak, et al. (2009) J Biol Chem 284(51): 35461-35470, and Koh, D. C., S. M. Wong, et al. (2003) J Biol Chem 278(23): 20565-20573, the content of which are hereby incorproated by reference in their entireties. Multi-gene expression constructs containing modified inteins have been disclosed in U.S. Pat. Nos. 7,026,526 and 7,741,530, as well as U.S. Patent application 2008/0115243, the content of which are hereby incorporated by reference in their entireties.

As used herein, the phrase “selectable marker” or “selectable marker gene” refers to a gene that is optionally used in plant transformation to, for example, protect the plant cells from a selective agent or provide resistance/tolerance to a selective agent. Only those cells or plants that receive a functional selectable marker are capable of dividing or growing under conditions having a selective agent. Examples of selective agents can include, for example, antibiotics, including spectinomycin, neomycin, kanamycin, paromomycin, gentamicin, and hygromycin. These selectable markers include gene for neomycin phosphotransferase (npt II), which expresses an enzyme conferring resistance to the antibiotic kanamycin, and genes for the related antibiotics neomycin, paromomycin, gentamicin, and G418, or the gene for hygromycin phosphotransferase (hpt), which expresses an enzyme conferring resistance to hygromycin. Other selectable marker genes can include genes encoding herbicide resistance including Bar (resistance against BASTA® (glufosinate ammonium), or phosphinothricin (PPT)), a.cetolactate synthase (ALS, resistance against inhibitors such as sulfonylureas (SUs), imidazolinones triazolopyrimidines (TPs), pyrimidinyl oxybenzoates (POBs), and sulfonylamino carbonyl triazolinones that prevent the first step in the synthesis of the branched-chain amino acids), glyphosate, 2,4-D, and metal resistance or sensitivity. The phrase “marker-positive” refers to plants that have been transformed to include the selectable marker gene.

Various selectable or detectable markers can be incorporated into the chosen expression vector to allow identification and selection of transformed plants, or transformants. Many methods are available to confirm the expression of selection markers in transformed plants, including for example DNA sequencing and PCR (polymerase chain reaction), Southern blotting, RNA blotting, immunological methods for detection of a protein expressed from the vector, e g., precipitated protein that mediates phosphinothricin resistance, or other proteins such as reporter genes β-glucuronidase (GUS), luciferase, green fluorescent protein (GFP), DsRed, β-galactosidase, chloramphenicol acetyltransferase (CAT), alkaline phosphatase, and the like (See Sambrook, et at, Molecular Cloning: A Laboratory Manual, Third Edition, Cold Spring Harbor Press, N.Y., 2001, the content of which is incorporated herein by reference in its entirety).

Selectable marker genes are utilized for the selection of transformed cells or tissues. Selectable marker genes include genes encoding antibiotic resistance, such as those encoding neomycin phosphotransferase II (NEO) and hygromycin phosphotransferase (HPT) as well as genes conferring resistance to herbicidal compounds. Herbicide resistance genes generally code for a modified target protein insensitive to the herbicide or for an enzyme that degrades or detoxifies the herbicide in the plant before it can act For example, resistance to glyphosate or has been obtained by using genes coding for the mutant target enzymes, 5-enolpyruvylshikimate-3-phosphate synthase (EPSPS). Genes and mutants for EPSPS have been disclosed in U.S. Pat. Nos. 4,940,835, 5,188,642, 5,310,667, 5,633,435, 5,633,448, and 6,566,587, the contents of which are incorporated by reference in their entireties. Resistance to glufosinate ammonium, bromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides. Enzymes/genes for glufosinate resistance/tolerance have been disclosed in U.S. Pat. Nos. 5,273,894, 5,276,268, 5,550,318, and 5,561,236, the contents of which are incorporated by reference in their entireties. Enzymes/genes for 2,4-D resistance have been previously disclosed in U.S. Pat. Nos. 6,100,446 and 6,153,401, as well as patent applications US 2009/0093366 and WO 2007/053482, the contents of which are hereby incorporated by reference in their entireties. Enzymes/genes for nitrilase has been previously disclosed in U.S. Pat. Nos. 4,810,648, the content of which is incorporated by reference in its entirety.

Other herbicides can inhibit the growing point or meristem, including imidazolinone or sulfonylurea, and genes for resistance/tolerance of acetohydroxyacid synthase (AHAS) and acetolactate synthase (ALS) for these herbicides have been described. Genes and mutants for AHAS and mutants have been disclosed in U.S. Pat. Nos. 4,761,373, 5,304,732, 5,331,107, 5,853,973, and 5,928,937, the contents of which are incorporated by reference in their entireties. Genes and mutants for ALS have been disclosed in U.S. Pat. Nos. 5,013,659 and 5,141,870, the contents of which are incorporated by reference in their entireties.

Glyphosate resistance genes include mutant 5-enolpyruvylshikimate-3-phosphate synthase (EPSPs) genes (via the introduction of recombinant nucleic acids and/or various forms of in viva mutagenesis of native EPSPs genes), aroA genes and glyphosate acetyl transferase (GAT) genes, respectively). Resistance genes for other phosphono compounds include glufosinate (phosphinothricin acetyl transferase (PAT) genes from Streptomyces species, including Streptomyces hygroscopicus and Streptomyces viridichromogenes), and pyridinoxy or phenoxy proprionic acids and cyclohexones (ACCase inhibitor-encoding genes). Herbicide resistance/tolerance genes of acetyl coemzyme A carboxylase (ACCas) have been described in U.S. Pat. Nos. 5,162,602 and 5,498,544, the contents of which are incorporated by reference in their entireties.

A DNA molecule encoding a mutant aroA gene can be obtained under ATCC accession number 39256, and the nucleotide sequence of the mutant gene is disclosed in U.S. Pat. No. 4,769,061 to Comai, European patent application No. 0 333 033 to Kumada et al., and U.S. Pat. No. 4,975,374 to Goodman et al., disclosing nucleotide sequences of glutamine synthetase genes which confer resistance to herbicides such as L-phosphinothricin. The nucleotide sequence of a PAT gene is provided in European application No. 0 242 246 to Leemans et al. Also DeGreef et al., BiolTechnology 7:61 (1989), describes the production of transgenic plants that express chimeric bar genes coding for PAT activity. Exemplary of genes conferring resistance to phenoxy proprionic acids and cyclohexones, including sethoxydim and haloxyfop, are the Acc1-S1, Ace1-52 and Acc1-53 genes described by Marshall et al. Theon. Appl. Genet. 83:435 (1992). GAT genes capable of conferring glyphosate resistance are described in WO 2005012515 to Castle et al. Genes conferring resistance to 2,4-D, fop and pyridyloxy auxin herbicides are described in WO 2005107437 and U.S. patent application Ser. No. 11/587,893.

Other herbicides can inhibit photosynthesis, including triazine (psbA and ls+ genes or benzonitrile (nitrilase gene). Przibila et al., Plant Cell 3:169 (1991), describes the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442, Cloning and expression of DNA coding for a glutathione S-transferase is described by Hayes et al., Biochem. J. 285:173 (1992).

For purposes of the present invention, selectable marker genes include, but are not limited to genes encoding: neomycin phosphotransferase II (Fraley et al. (1986) CRC Critical Reviews in Plant Science, 4:1-25); cyanamide hydratase (Maier-Greiner et al. (1991) Proc. Natl. Acad. Sci. USA, 88:4250-4264); aspartate kinase; dihydrodipicolinate synthase (Peri et al. (1993) Bio/Technology, 11:715-718); tryptophan decarboxylase (Goddijn et al. (1993) Plant Mol. Bio., 22:907-912); dihydrodipicolinate synthase and desensitized aspartade kinase (Peri et al. (1993) Bio/Technology, 11:715-718); bar gene (Toki et al. (1992) Plant Physiol., 100:1503-1507 and Meagher et al. (1996) and Crop Sci., 36:1367); tryptophan decarboxylase (Goddijn et al. (1993) Plant Mol. Biol., 22:907-912); neomycin phosphotransferase (NEO) (Southern et al. (1982) J. Mol. Appl. Gen., 1:327; hygromycin phosphotransferase (EIPT or HYG) (Shimizu et al, (1986) Mol. Cell Biol., 6:1074); dihydrofolate reductase (DHFR) (Kwok et al. (1986) PNAS USA 4552); phosphinothricin acetyltransferase (DeBlock et al. (1987) EMBO J., 6:2513); 2,2-dichloropropionic acid dehalogenase (Buchanan-Wollatron et al. (1989) J. Cell. Biochem. 13D: 330); acetohydroxyacid synthase (Anderson et al., U.S. Pat. No. 4,761,373; Haughn et al. (1988) Mol. Gen. Genet. 221:266); 5-enolpyruvyl-shikimate-phosphate synthase (aroA) (Comai et al. (1985) Nature 317:741); haloarylnitrilase (Stalker et al., published PCT application WO87/04181); acetyl-coenzyme A carboxylase (Parker et al. (1990) Plant Physiol. 92:1220); dihydropteroate synthase (sul I) (Guerineau et al. (1990) Plant Mol. Biol. 15:127); and 32 kD photosystem II polypeptide (psbA) (Hirschberg et al. (1983) Science, 222:1346).

Also included are genes encoding resistance to: chloramphenicol (Herrera-Estrella et al. (1983) EMBO J., 2:987-992); methotrexate (Herrera-Estrella et al. (1983) Nature, 303:209-213; Meijer et al. (1991) Plant Mol Bio., 16:807-820 (1991); hygromycin (Waldron et al. (1985) Plant Mol. Biol., 5:103-108; Zhijian et al, (1995) Plant Science, 108:219-227 and Meijer et al. (1991) Plant Mol. Bio. 16:807-820); streptomycin (Jones et al. (1987) Mol. Gen. Genet., 210:86-91); spectinomycin (Bretagne-Sagnard et al. (1996) Transgenic Res., 5:131-137); bleomycin (Hille et al. (1986) Plant Mol. Biol., 7:171-176); sulfonamide (Guerineau et al. (1990) Plant Mol. Bio., 15:127-136); bromoxynil (Stalker et al. (1988) Science, 242:419-423); 2,4-D (Streber et al. (1989) Bio/Technology, 7:811-816); glyphosate (Shaw et al. (1986) Science, 233:478-481); and phosphinothricin (DeBlock et al. (1987) EMBO J., 6:2513-2518). All references recited in the disclosure are hereby incorporated by reference in their entireties unless stated otherwise.

The above list of selectable marker and reporter genes are not meant to be limiting. Any reporter or selectable marker gene are encompassed by the present invention. If necessary, such genes can be sequenced by methods known in the art.

The reporter and selectable marker genes are synthesized for optimal expression in the plant. That is, the coding sequence of the gene has been modified to enhance expression in plants. The synthetic marker gene is designed to be expressed in plants at a higher level resulting in higher transformation efficiency. Methods for synthetic optimization of genes are available in the art. In fact, several genes have been optimized to increase expression of the gene product in plants.

The marker gene sequence can be optimized for expression in a particular plant species or alternatively can be modified for optimal expression in plant families. The plant preferred codons may be determined from the codons of highest frequency in the proteins expressed in the largest amount in the particular plant species of interest. See, for example, EPA 0359472; EPA 0385962.; WO 91/16432.; Perlak et al. (1991) Proc. Natl. Acad. Sci. USA, 88:3324-3328; and Murray et al. (1989) Nucleic Acids Research, 17: 477-498; U.S. Pat. No. 5,380,831; and U.S. Pat. No. 5,436,391, herein incorporated by reference. In this manner, the nucleotide sequences can be optimized for expression in any plant. It is recognized that all or any part of the gene sequence may be optimized or synthetic. That is, fully optimized or partially optimized sequences may also be used.

Genes that Confer Resistance to an Herbicide:

A. Resistance/tolerance of acetohydroxyacid synthase (AHAS) and acetolactate synthase (ALS) against herbicides imidazolinone or sulfonylurea. Genes and mutants for AHAS and mutants have been disclosed in U.S. Pat. Nos. 4,761,373, 5,304,732, 5,331,107, 5,853,973, and 5,928,937. Genes and mutants for ALS have been disclosed in U.S. Pat. Nos. 5,013,659 and 5,141,870.

B. Resistance/tolerance genes of acetyl coemzyme A carboxylase (ACCas) against herbicides cyclohexanediones and/or aryloxyphenoxypropanoic acid (including Haloxyfop, Diclofop, Fenoxyprop, Fluazifop, Quizalopfop) have been described in U.S. Pat. Nos. 5,162,602 and 5,498,544.

C. Genes for glyphosate resistance/tolerance. Gene of 5-enolpyruvyl-3-phosphoshikimate synthase (ES3P synthase) has been described in U.S. Pat. No. 4,769,601, Genes of 5-enolpyruvylshikimate-3-phosphate synthase (EPSPS) and mutants have been described in U.S. Pat. Nos. 4,940,835, 5,188,642, 5,310,667, 5,633,435, 5,633,448, and 6,566,587.

D. Genes for glufosinate (bialaphos, phosphinathficin (PPT)) resistance/tolerance. Gene for phosphinothricin acetyltransferase (Pat) has been described in U.S. Pat. Nos. 5,273,894, 5,276,268, and 5,550,318; and gene for bialaphos resistance gene (Bar) has been described in U.S. Pat. Nos. 5,561,236 and 5,646,024, 5,648,477, and 7,112,665. Gene for glutamine synthetase (GS) has been described in U.S. Pat. No. 4,975,372 and European patent application EP 0333033 A1.

E. Resistance/tolerance genes of hydroxy phenyl pyruvate dioxygenase (HPPD) against herbicides isoxazole, diketonitriles, and/or tri ketones including sulcotrione and mesotrione have been described in U.S. Pat. Nos. 6,268,549 and 6,069,115.

F. Genes for 2,4-D resistance/tolerance. Gene of 2,4-D-monooxygenase has been described in U.S. Pat. Nos. 6,100,446 and 6,153,401. Additional genes for 2,4-D resistance/tolerance are disclosed in US 2009/0093366 and WO 2007/053482,

G. Gene of imidazoleglycerol phosphate dehydratase (IGPD) against herbicides imidazole and/or triazole has been described in U.S. Pat. No. 5,541,310. Genes of Dicamba degrading enzymes (oxygenase, ferredoxin, and reductase) against herbicide Dicamba have been disclosed in U.S. Pat. Nos. 7,022,896 and 7,105,724.

H. Genes for herbicides that inhibit photosynthesis, including triazine (psbA and ls+ genes) or a benzonitrile (nitrilase gene). See e.g., Przibila et at, Plant Cell 3:169 (1991) disclosing transformation of Chlamydomonas with plasmids encoding mutant psbA genes. Nucleotide sequences for nitrilase genes are disclosed in U.S. Pat. No. 4,810,648 and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

Unless otherwise specifically explained, all technical and scientific terms used herein have the same meaning as commonly understood by those of ordinary skill in the art to which this disclosure belongs. Definitions of common terms in molecular biology can be found in, for example: Lewin, Genes V, Oxford University Press, 1994 (ISBN 0-19-854287-9); Kendrew et al. (eds.), The Encyclopedia of Molecular Biology, Blackwell Science Ltd. 1994 (ISBN 0-632-02182-9); and Meyers (ed.), Molecular Biology and Biotechnology: A Comprehensive Desk Reference, VCH Publishers, Inc., 1995 (ISBN 1-56081-569-8).

Provided are nucleic acid molecules comprising a synthetic nucleotide sequence that may function as a bidirectional promoter. In some embodiments, a synthetic bidirectional promoter may be operably linked to one or two nucleotide sequence(s) of interest. For example, a synthetic bidirectional promoter may be operably linked to one or two nucleotide sequence(s) of interest (e.g., two genes, one on each end of the promoter), so as to regulate transcription of at least one (e.g., one or both) of the nucleotide sequence(s) of interest. By incorporating a URS from a promoter in the synthetic bidirectional promoter, particular expression and regulatory patterns (e.g., such as are exhibited by genes under the control of the native promoter) may be achieved with regard to a nucleotide sequence of interest that is operably linked to the synthetic bidirectional promoter.

Some embodiments of the invention are exemplified herein by incorporating a minimal core promoter element from a unidirectional maize ubiquitin-1 gene (ZMUbi1) promoter into a molecular context different from that of the native promoter to engineer a synthetic bidirectional promoter. This minimal core promoter element is referred to herein as “minUbi1P,” and is approximately 200 nt in length. Sequencing and analysis of minUbi1P elements from multiple Zea species and Z. mays genotypes has revealed that functional minUbi1P elements are highly conserved, such that a minUbi1P element may preserve its function as an initiator of transcription if it shares, for example, at least about 75%; at least about 80%; at least about 85%; at least about 90%, at least about 91%; at least about 92%; at least about 93%, at least about 94%; at least about 95%, at least about 96%; at least about 97%; at least about 98%; at least about 99%; and/or at least about 100% sequence identity to the mintihil P element of SEQ ID NO:1. Characteristics of minUbi1P elements that may be useful in some embodiments of the invention may include, for example and without limitation, the aforementioned high conservation of nucleotide sequence; the presence of at least one TATA box; and/or the presence of at least one (e.g., two) heat shock consensus element(s). In particular minUbi IP elements, more than one heat shock consensus elements may be overlapping within the mintibilP sequence.

The process of incomorating a minUbi1P element into a molecular context different from that of a native promoter to engineer a synthetic bidirectional promoter may comprise reversing the orientation of the minUbi1P element in a nucleic acid with respect to the remaining sequence of the promoter, including its native minimal core promoter. Thus, a synthetic bidirectional promoter may comprise a first minUbi1P element incorporated 5′ of a second minimal core promoter element (e.g., a second minUbi1P element) in the promoter in the reverse orientation, such that it may be operably linked to a nucleotide sequence of interest located 5′ of the first minUbi1P element. For example, the first minUbi1P element may be incorporated at the 5′ end of a ZmUbi1 promoter in reverse orientation.

A synthetic bidirectional Ubi1 promoter may also comprise one or more additional sequence elements in addition to at least one minUbi1P element. In some embodiments, a synthetic bidirectional Ubi1 promoter may comprise a promoter URS; an exon (e.g., a leader or signal peptide); an intron; a spacer sequence; and or combinations of one or more of any of the foregoing. For example and without limitation, a synthetic bidirectional Ubi1 promoter may comprise a URS sequence from a Ubi1 promoter (e.g., the maize Ubi1 promoter); an exon encoding a leader peptide from a Ubi1 gene; an intron from a Ubi1 gene; and combinations of these.

In some of those examples comprising a synthetic bidirectional Ubi1 promoter comprising a promoter URS, the URS may be selected to confer particular regulatory properties on the synthetic promoter. Known promoters vary widely in the type of control they exert on operably linked genes (e.g., environmental responses, developmental cues, and spatial information), and a URS incorporated into a heterologous promoter typically maintains the type of control the URS exhibits with regard to its native promoter and operably linked gene(s). Langridge et al. (1989), supra. Examples of eukaryotic promoters that have been characterized and may contain a URS comprised within a synthetic bidirectional Ubi1 promoter according to some embodiments include, for example and without limitation: those promoters described in U.S. Pat. No. 6,437,217 (maize RS81 promoter); U.S. Pat. No. 5,641,876 (rice actin promoter); U.S. Pat. No. 6,426,446 (maize RS324 promoter); U.S. Pat. No. 6,429,362 (maize PR-1 promoter); U.S. Pat. No. 6,232,526 (maize A3 promoter); U.S. Pat. No. 6,177,611 (constitutive maize promoters); U.S. Pat. No. 6,433,252 (maize L3 oleosin promoter); U.S. Pat. No. 6,429,357 (rice actin 2 promoter, and rice actin 2 intron); U.S. Pat. No. 5,837,848 (root-specific promoter); U.S. Pat. No. 6,294,714 (light-inducible promoters); U.S. Pat. No. 6,140,078 (salt-inducible promoters); U.S. Pat. No. 6,252,138 (pathogen-inducible promoters); U.S. Pat. No. 6,175,060 (phosphorous deficiency-inducible promoters); U.S. Pat. No. 6,388,170 (bidirectional promoters); U.S. Pat. No. 6,635,806 (gamma-coixin promoter); and U.S. patent application Ser. No. 09/757,089 (maize chloroplast aldolase promoter).

Additional exemplary prokaryotic promoters include the nopaline synthase (NOS) promoter (Ebert et al. (1987) Proc. Natl. Acad. Sci, USA 84(16):5745-9); the octopine synthase (OCS) promoter (which is carried on tumor-inducing plasmids of Agrobacterium tumelitciens); the caulimovirus promoters such as the cauliflower mosaic virus (CaMV) 19S promoter (Lawton et al. (1987) Plant Mol. Biol. 9:315-24); the CAMV 35S promoter (Odell et al. (1985) Nature 313:810-2; the figwort mosaic virus 35S-promoter (Walker et al. (1987) Proc. Natl. Acad. Sci. USA 84(19):6624-8); the sucrose synthase promoter (Yang and Russell (1990) Proc. Natl. Acad. Sci. USA 87:4144-8); the R gene complex promoter (Chandler et al. (1989) Plant Cell 1:1175-83); CaMV35S (U.S. Pat. Nos. 5,322,938, 5,352,605, 5,359,142, and 5,530,196); FMV35S (U.S. Pat. Nos. 6,051,753, and 5,378,619); a PC1SV promoter (U.S. Pat. No. 5,850,019); the SCP1 promoter (U.S. Pat. No. 6,677,503); and AGRtu.nos promoters (GenBank Accession No. V00087; Depicker et al. (1982) J. Mol. Appl. Genet. 1:561-73; Bevan et al. (1983) Nature 304:184-7), and the like.

In some embodiments, a synthetic bidirectional Ubi1 promoter may further comprise an exon in addition to minUbi1P element(s). For example, it may be desirable in particular embodiments to target or traffic a polypeptide encoded by a nucleotide sequence of interest operably linked to the promoter to a particular subcellular location and/or compartment. In these and other embodiments, a coding sequence (exon) may be incorporated into a nucleic acid molecule between the minUbi1P element and a nucleotide sequence encoding a polypeptide. These elements may be arranged according to the discretion of the skilled practitioner such that the synthetic bidirectional Ubi1 promoter promotes the expression of a polypeptide (or one or both of two polypeptide-encoding sequences that are operably linked to the promoter) comprising the peptide encoded by the incorporated coding sequence in a functional relationship with the remainder of the polypeptide. In particular examples, an exon encoding a leader, transit, or signal peptide (e.g., the Ubi1 leader peptide) may be incorporated.

Peptides that may be encoded by an exon incorporated into a synthetic bidirectional Ubi1 promoter include, for example and without limitation: a Ubiquitin (e.g., Ubi1) leader exon; and a chloroplast transit peptide (CTP) (e.g., the A. thahana EPSPS CTP (Klee et al. (1987) Mol. Gen. Genet. 210:437-42), and the Petunia hybrida EPSPS CTP (della-Cioppa et a/. (1986) Proc. Natl. Acad. Sci, USA 83:6873-7)), as exemplified for the chloroplast targeting of dicamba monooxygenase (DMO) in International PCT Publication No. WO 2008/105890.

Introns may also be incorporated in a synthetic bidirectional Ubi1 promoter in some embodiments of the invention, for example, between a minUbi1P element and a nucleotide sequence of interest that is operably linked to the promoter. In some examples, an intron incorporated into a synthetic bidirectional Ubi1 promoter may be, without limitation, a 5′ UTR that functions as a translation leader sequence that is present in a fully processed mRNA upstream of the translation start sequence (such a translation leader sequence may affect processing of a primary transcript to mRNA, mRNA stability, and/or translation efficiency). Examples of translation leader sequences include maize and petunia heat shock protein leaders (U.S. Pat. No. 5,362,865), plant virus coat protein leaders, plant rubisco leaders, and others. See, e.g., Turner and Foster (1995) Molecular Biotech. 3(3):225-36. Non-limiting examples of 5′ UTRs include GmHsp (U.S. Pat. No. 5,659,122); PhDnaK (U.S. Pat. No. 5,362,865); AtAnt1; TEV (Carrington and Freed (1990) J. Virol. 64:1590-7); and AGRtu.nos (GenBank Accession No. V00087; and Bevan et al. (1983) Nature 304:184-7).

Additional sequences that may optionally be incorporated into a synthetic bidirectional Ubi1 promoter include, for example and without limitation:. 3′ non-translated sequences; 3′ transcription termination regions; and polyadenylation regions. These are genetic elements located downstream of a nucleotide sequence of interest (e.g., a sequence of interest that is operably linked to a synthetic bidirectional Ubi1 promoter), and include polynucleotides that provide polyadenylation signal, and/or other regulatory signals capable of affecting transcription, mRNA processing, or gene expression. A polyadenylation signal may function in plants to cause the addition of polyadenylate nucleotides to the 3′ end of a mRNA precursor. The polyadenylation sequence may be derived from the natural gene, from a variety of plant genes, or from T-DNA genes. Anon-limiting example of a 3′ transcription termination region is the nopaline synthase 3′ region (nos 3′; Fraley et at (1983) Proc. Natl. Acad, Sci. USA 80:4803-7). An example of the use of different 3′ nontranslated regions is provided in Ingelbrecht et al., (1989) Plant Cell 1:671-80. Non-limiting examples of polyadenylation signals include one from a Pisum sativum RbcS2 gene (PsRbeS2-E9; Coruzzi et al. (1984) EMBO J. 3:1671-9) and AGRtu.nos (GenBank Accession No. E01312).

In some embodiments, a synthetic bidirectional Ubi1 promoter comprises one or more nucleotide sequences that facilitate targeting of a nucleic acid comprising the promoter to a particular locus in the genome of a target organism. For example, one or more sequences may be included that are homologous to segments of genomic DNA sequence in the host (e.g., rare or unique genomic DNA sequences). In some examples, these homologous sequences may guide recombination and integration of a nucleic acid comprising a synthetic bidirectional Ubi1 promoter at the site of the homologous DNA in the host genome. In particular examples, a synthetic bidirectional Ubi1 promoter comprises one or more nucleotide sequences that facilitate targeting of a nucleic acid comprising the promoter to a rare or unique location in a host genome utilizing engineered nuclease enzymes that recognize sequence at the rare or unique location and facilitate integration at that rare or unique location. Such a targeted integration system employing zinc-finger endonucleases as the nuclease enzyme is described in U.S. patent application Ser. No. 13/011,735, the contents of the entirety of which are incorporated herein by this reference.

Nucleic acids comprising a synthetic bidirectional Ubi1 promoter may be produced using any technique known in the art, including for example and without limitation: RCA; PCR amplification; RT-PCR amplification; OLA; and SNuPE. These and other equivalent techniques are well known to those of skill in the art, and are further described in detail in, for example and without limitation: Sambrook et al. Molecular Cloning: A Laboratory Manual, 3′ Ed., Cold Spring Harbor Laboratory, 2001; and Ausubel et al. Current Protocols in Molecular Biology, John Wiley & Sons, 1998, All of the references cited above, including both of the foregoing manuals, are incorporated herein by this reference in their entirety, including any drawings, figures, and/or tables provided therein.

Delivery and/or transformation: The present disclosure also provides methods for transforming a cell with a nucleic acid molecule comprising a synthetic bidirectional UN1 promoter. Any of the large number of techniques known in the art for introduction of nucleic acid molecules into plants may be used to transform a plant with a nucleic acid molecule comprising a synthetic bidirectional tibit promoter according to some embodiments, for example, to introduce one or more synthetic bidirectional Ubi1 promoters into the host plant genome, and/or to further introduce one or more nucleic acid molecule(s) of interest operably linked to the promoter.

Suitable methods for transformation of plants include any method by which DNA can be introduced into a cell, for example and without limitation: electroporation (see, e.g., U.S. Pat. No. 5,384,253); microprojectile bombardment (see, e.g., U.S. Pat. Nos. 5,015,580, 5,550,318, 5,538,880, 6,160,208, 6,399,861, and 6,403,865); Agrobacterium-mediated transformation (see, e.g., U.S. Pat. Nos. 5,635,055, 5,824,877, 5,591,616; 5,981,840, and 6,384,301); and protoplast transformation (see, e.g., U.S. Pat. No. 5,508,184). Through the application of techniques such as the foregoing, the cells of virtually any plant species may be stably transformed, and these cells may be developed into transgenic plants by techniques known to those of skill in the art. For example, techniques that may be particularly useful in the context of cotton transformation are described in U.S. Pat. Nos. 5,846,797, 5,159,135, 5,004,863, and 6,624,344; techniques for transforming Brassica plants in particular are described, for example, in U.S. Pat. No. 5,750,871; techniques for transforming soya are described, for example, in U.S. Pat. No. 6,384,301; and techniques for transforming maize are described, for example, in U.S. Pat. Nos. 7,060,876 and 5,591,616, and international PCT Publication WO 95/06722.

After effecting delivery of an exogenous nucleic acid to a recipient cell, the transformed cell is generally identified for further culturing and plant regeneration. In order to improve the ability to identify transformants, one may desire to employ a selectable or screenable marker gene with the transformation vector used to generate the transformant. In this case, the potentially transformed cell population can be assayed by exposing the cells to a selective agent or agents, or the cells can be screened for the desired marker gene trait.

Cells that survive the exposure to the selective agent, or cells that have been scored positive in a screening assay, may be cultured in media that supports regeneration of plants. In some embodiments, any suitable plant tissue culture media (e.g., MS and N6 media) may be modified by including further substances, such as growth regulators. Tissue may be maintained on a basic media with growth regulators until sufficient tissue is available to begin plant regeneration efforts, or following repeated rounds of manual selection, until the morphology of the tissue is suitable for regeneration (e.g., at least 2 weeks), then transferred to media conducive to shoot formation. Cultures are transferred periodically until sufficient shoot formation has occurred. Once shoots are formed, they are transferred to media conducive to root formation. Once sufficient roots are formed, plants can be transferred to soil for further growth and maturity.

To confirm the presence of the desired nucleic acid molecule comprising a synthetic bidirectional UN1 promoter in the regenerating plants, a variety of assays may be performed. Such assays include, for example: molecular biological assays, such as Southern and Northern blotting and PCR; biochemical assays, such as detecting the presence of a protein product, e.g., by immunological means (ELISA and/or Western blots) or by enzymatic function; plant part assays, such as leaf or root assays; and analysis of the phenotype of the whole regenerated plant.

Targeted integration events may be screened, for example, by PCR amplification using, e.g., oligonucleotide primers specific for nucleic acid molecules of interest. PCR genotyping is understood to include, but not be limited to, polymerase-chain reaction (PCR) amplification of genomic DNA derived from isolated host plant callus tissue predicted to contain a nucleic acid molecule of interest integrated into the genome, followed by standard cloning and sequence analysis of PCR amplification products. Methods of PCR genotyping have been well described (see, e.g., Rios et al. (2002) Plant J. 32:243-53), and may be applied to genomic DNA derived from any plant species or tissue type, including cell cultures. Combinations of oligonucleotide primers that bind to both target sequence and introduced sequence may be used sequentially or multiplexed in PCR amplification reactions. Oligonucleotide primers designed to anneal to the target site, introduced nucleic acid sequences, and/or combinations of the two may be produced. Thus, PCR genotyping strategies may include, for example and without limitation: amplification of specific sequences in the plant genome; amplification of multiple specific sequences in the plant genome; amplification of non-specific sequences in the plant genome; and combinations of any of the foregoing. One skilled in the art may devise additional combinations of primers and amplification reactions to interrogate the genome. For example, a set of forward and reverse oligonucleotide primers may be designed to anneal to nucleic acid sequence(s) specific for the target outside the boundaries of the introduced nucleic acid sequence.

Forward and reverse oligonucleotide primers may be designed to anneal specifically to an introduced nucleic acid molecule, for example, at a sequence corresponding to a coding region within a nucleotide sequence of interest comprised therein, or other parts of the nucleic acid molecule. These primers may be used in conjunction with the primers described above. Oligonucleotide primers may be synthesized according to a desired sequence, and are commercially available (e.g., from Integrated DNA. Technologies, Inc., Coralvllne, Iowa). Amplification may be followed by cloning and sequencing, or by direct sequence analysis of amplification products. One skilled in the art might envision alternative methods for analysis of amplification products generated during PCR genotyping. In one embodiment, oligonucleotide primers specific for the gene target are employed in PCR amplifications.

Some embodiments of the present invention also provide cells comprising a synthetic bidirectional Ubi1 promoter, for example, as may be present in a nucleic acid construct. In particular examples, a synthetic bidirectional Ubi1 promoter according to some embodiments may be utilized as a regulatory sequence to regulate the expression of transgenes in plant cells and plants. In some such examples, the use of a synthetic bidirectional Ubi1 promoter operably linked to a nucleotide sequence of interest (e.g., a transgene) may reduce the number of homologous promoters needed to regulate expression of a given number of nucleotide sequences of interest, and/or reduce the size of the nucleic acid construct(s) required to introduce a given number of nucleotide sequences of interest. Furthermore, use of a synthetic bidirectional Ubi1 promoter may allow co-expression of two operably linked nucleotide sequence of interest under the same conditions (i.e., the conditions under which the promoter is active). Such examples may be particularly useful, e.g., when the two operably linked nucleotide sequences of interest each contribute to a single trait in a transgenic host comprising the nucleotide sequences of interest, and co-expression of the nucleotide sequences of interest advantageously impacts expression of the trait in the transgenic host.

In some embodiments, a transgenic plant comprising one or more synthetic bidirectional Ubi1 promoter(s) and/or nucleotide sequence(s) of interest may have one or more desirable traits conferred (e.g., introduced, enhanced, or contributed to) by expression of the nucleotide sequence(s) of interest in the plant. Such traits may include, for example and without limitation: resistance to insects, other pests, and disease-causing agents; tolerances to herbicides; enhanced stability, yield, or shelf-life; environmental tolerances; pharmaceutical production; industrial product production; and nutritional enhancements. In some examples, a desirable trait may be conferred by transformation of a plant with a nucleic acid molecule comprising a synthetic bidirectional Ubi1 promoter operably linked to a nucleotide sequence of interest. In some examples, a desirable trait may be conferred to a plant produced as a progeny plant via breeding, which trait may be conferred by one or more nucleotide sequences of interest operably linked to a synthetic bidirectional Ubi1 promoter that is/are passed to the plant from a parent plant comprising a nucleotide sequence of interest operably linked to a synthetic bidirectional Ubi1 promoter.

A transgenic plant according to some embodiments may be any plant capable of being transformed with a nucleic acid molecule of the invention, or of being bred with a plant transformed with a nucleic acid molecule of the invention. Accordingly, the plant may be a dicot or monocot. Non-limiting examples of dicotyledonous plants for use in some examples include: alfalfa; beans; broccoli; canola, cabbage; carrot; cauliflower; celery; Chinese cabbage; cotton; cucumber; eggplant; lettuce; melon; pea; pepper; peanut; potato; pumpkin; radish; rapeseed; spinach; soybean; squash; sugarbeet; sunflower; tobacco; tomato; and watermelon. Non-limiting examples of monocotyledonous plants for use in some examples include: corn; onion; rice; sorghum; wheat; rye; millet; sugarcane; oat; triticale; switchgrass; and turfgrass.

In some embodiments, a transgenic plant may be used or cultivated in any manner, Wherein presence a synthetic bidirectional Ubi1 promoter and/or operably linked nucleotide sequence of interest is desirable. Accordingly, such transgenic plants may be engineered to, inter cilia, have one or more desired traits, by being transformed with nucleic acid molecules according to the invention, and may be cropped and/or cultivated by any method known to those of skill in the art.

While the invention has been described with reference to specific methods and embodiments, it will be appreciated that various modifications and changes may be made without departing from the invention. All publications cited herein are expressly incorporated herein by reference for the purpose of describing and disclosing compositions and methodologies that might be used in connection with the invention. All cited patents, patent applications, and sequence information in referenced websites and public databases are also incorporated by reference.

The following examples are provided to illustrate certain particular features and/or embodiments. The examples should not be construed to limit the disclosure to the particular features or embodiments exemplified.

### EXAMPLES

### Example 1

### Transformation and Expression

Transformation of Agrobacterium tumetaciens: The pDAB108706 binary vector is transformed into Agrobacterium tumefaciens strain DAt13192 ternary (U.S. Prov. Pat. No. 61/368965). Bacterial colonies are isolated and binary plasmid DNA is isolated and confirmed via restriction enzyme digestion.

Corn Transformation: Ear Sterilization and Embryo Isolation. To obtain maize immature embryos, plants of Zea mays (c.v. B104) are grown in the greenhouse and self or sib-pollinated to produce ears. The ears are harvested approximately 9-12 days post-pollination. On the day of the experiment, ears are surface-sterilized by immersion in a 20% solution of household bleach, which contains 5% sodium hypochlorite, and shaken for 20-30 minutes, followed by three rinses in sterile water. After sterilization, immature zygotic embryos (1.5-2.2 mm) are aseptically dissected from each ear and randomly distributed into micro-centrifuge tubes containing liquid infection media (LS Basal Medium, 4.43 gm/L; N6 Vitamin Solution [1000×], 1.00 mL/L, L-proline, 700.0 mg/L; sucrose, 68.5 gm/L, glucose, 36.0 gm/L; 2,4-D, 1.50 mg/L. For a given set of experiments, pooled embryos from 2-3 ears are used for each treatment.

Agrobacterium Culture Initiation: Glycerol stocks of Agrobacterium containing the binary vectors described above are streaked on AB minimal medium plates containing appropriate antibiotics and are grown at 20° C. for 3-4 days. A single colony is picked and streaked onto YEP plates containing the same antibiotics and was incubated at 28° C. for 1-2 days.

Agrobacterium Culture and Co-cultivation: On the day of the experiment, Agrobacterium colonies are taken from the YEP plate, suspended in 10 mL of infection medium in a 50 mL disposable tube, and the cell density is adjusted to OD600=0.2-0.4 nm using a spectrophotometer. The Agrobacterium cultures are placed on a rotary shaker at 100 rpm, room temperature. While embryo dissection is performed. Immature zygotic embryos between 1.5-2.2 mm in size are isolated from the sterilized maize kernels and placed in 1 mL of the infection medium and washed once in the same medium. The Agrobacterium suspension (2 mL) is added to each tube and the tubes are inverted for about 20 times then shaken for 10-15 minutes. The embryos are transfected onto co-cultivation media (MS Salts, 4.33 gm/L; L-proline, 700.0 mg/L; myo-inositol, 100.0 mg/1₄ casein enzymatic hydrolysate 100.0 mg/L; Dicamba-3.30 mg/L; sucrose. 30.0 gm/L; Gelzan™, 3.00 gm/L; modified MS-Vitamin [1000×], 1.00 ml/L, AgNo₃, 15.0 mg/L; Acetosyringone, 100 μM), oriented with the scuteltum facing up, and incubated for 3-4 days in the light at 25° C.

GUS and YFP/Phiyfp Transient expression: Transient YFP/Phiyfp and GUS expression can be observed in transformed embryos and after 3 days of co-cultivation with Agrobacterium. The embryos are observed under a stereomicroscope (Leica. Microsystems, Buffalo Grove, Ill.) using YFP filter and 500 nm light source. Embryos showing YFP/Phiyfp expression are selected for GUS histochemical assay. GUS staining solution is prepared as described in Maniatis et al. (1989) and embryos are incubated in 1 mL solution for 24 hours at 37° C. The embryos are observed for GUS transient expression under the microscope.

Callus Selection and Regeneration of Putative Events: Following the co-cultivation period, embryos are transferred to resting media (MS salts, 4.33 gm/L; L-proline, 700.0 mg/L; myo-inositol, 100.0 mg/L; MES [(2-(n-morpholino)-ethanesulfonic acid), free acid] 500.0 mg/L; casein enzymatic hydrolysate 100.0 mg/L; Dicamba, 3.30 mg/L; sucrose, 30.0 gm/L; Gelzan 2.30 gm/L; modified MS-Vitamin [1000×], 1.00 ml/L; AgNo₃, 15.0 mg/L; Carbenicillin, 250.0 mg/L) without selective agent and incubated in 24 hours light with light intensity of 50 μmol⁻¹s⁻¹ for 7 days at 28° C. Embryos are transferred onto selection 1 media (MS salts, 4.33 gm/L; L-proline, 700.0 mg/L; myo-inositol, 100.0 mg/L; MES [(2-(n-morpholino)-ethanesulfonic acid), free acid] 500.0 mg/L; casein enzymatic kydrolysate 100.0 mg/L; Dicatnba, 3.30 mg/L; sucrose, 30.0 gm/L; Gelzan™ 2.30 gm/L; modified MS-Vitamin [1000×], 1.00 ml/L; AgNo₃, 15.0 mg/L, Carbenicillin, 250.0 mg/L) containing 100 nM haloxyfop and incubated in 24 hours light with light intensity of 50 μmol m⁻²s⁻¹ for 7 days at 28° C.

Embryos with proliferating embryogenic calli are transferred onto selection 2 media (MS salts, 4.33 gm/L; myo-inositol, 100.0 mg/L; L-proline, 700.0 mg/L; MES [(2-(n-morpholino)-ethanesulfonic acid), free acid] 500.0 mg/L; casein enzymatic hydrolysate 100.0 mg/L; Dicamba, 3.30 mg/L; sucrose, 30.0 gm/L; Gelzan™ 2.30 gm/L; modified MS-Vitamin [1000×], 1.00 ml/L; AgNo₃, 15.0 mg/L; Carbenicillin, 250.0 mg/L) containing 500 nM haloxyfop and are incubated in 24 hours light with light intensity of 50 μmol m⁻²s⁻¹ for another 14 days at 28° C. This selection step allows transgenic callus to further proliferate and differentiate. The callus selection period lasts for three weeks. Proliferating, embryogenic calli are transferred onto regeneration 1 media (MS salts, 4.33 gm/L; myo-inositol, 100.0 mg/L; L-proline, 350.0 mg/L; MES [(2-(n-morpholino)-ethanesulfonic acid), free acid] 250.0 mg/L; casein enzymatic hydrolysate 50.0 mg/L; NAA 0.500 mg/L; ABA 2.50 mg/L; BA 1.00 mg/L; sucrose, 45.0 gm/L; Gelzan™ 2.50 gm/L; modified MS-Vitamin [1000×], 1.00 ml/L; AgNo₃, 1.00 mg/L; Carbenicillin, 250.0 mg/L) containing 500 nM haloxythp and cultured in 24 hours light with light intensity of 50 μmol m⁻²s⁻¹ for 7 days at 28° C. Embryogenic calli with shoot/buds are transferred onto regeneration 2 media (MS salts, 4.33 gm/L; modified MS-Vitamin [1000×], 1.00 ml/L; myo-inositol, 100.0 mg/L; sucrose, 60.0 gm/L; Gellan Gum ^{G}434™ 3.00 gm/L; Carbenicillin, 250.0 mg/L) containing 500 nM haloxyfop. The cultures are incubated under 24 hours light with light intensity of 50 μmol m⁻²s⁻¹ for 7-10 days at 28° C. Small shoots with primary roots are transferred to shoot elongation and rooting media (MS salts, 4.33 gm/L; modified MS-Vitamin [1000×], 1.00 ml/L; myo-inositol, 100.0 mg/L; sucrose, 60.0 gm/L; Gellan Gum G434™ 3.00 gm/L; Carbenicillin, 250.0 mg/L) in MAGENTA™ boxes (Sigma-Aldrich, St. Louis, Mo.), and are incubated under 16/8 hours light/dark for 7 days at 28° C. Putative transgenic plantlets are analyzed for transgene copy number and transferred to the greenhouse.

### Example 2

### Construction of Synthetic Bidirectional Ubi1 Promoter and pDAB108706 Vector

An exemplary schematic drawing of the maize Ubiquitin-1 promoter (Ubi1) is shown in FIG. 1. An Ubi1 promoter is cloned from maize. A plasmid containing the Ubi1 promoter is PR amplified using a high-fidelity PCR amplification system. An approximately 200 nt region of the maize Ubi1 promoter is identified as a Zea mays Ubi1 minimal core promoter (minUbi1V (SEQ ID NO: 1). The minUbi1P of SEQ ID NO: 1 is then added to a polynucleotide (SEQ ID NO: 2) comprising a Zea mays Ubiquitin-1 exon (ZmUbi1 exon) and Zea mays Ubiquitin-1 intron (ZmUbi1intron) using cloning methods commonly known in the art to produce the polynucleotide of SEQ ID NO: 3. The resulting polynucleotide is then cloned upstream in reverse orientation of a nucleic acid comprising the maize Ubi1 promoter (including the Ubi1 upstream regulatory sequence (URS)); SEQ ID NO: 4) to produce the synthetic bidirectional Ubi1 promoter of SEQ ID NO: 5 (see FIG. 5 for example).

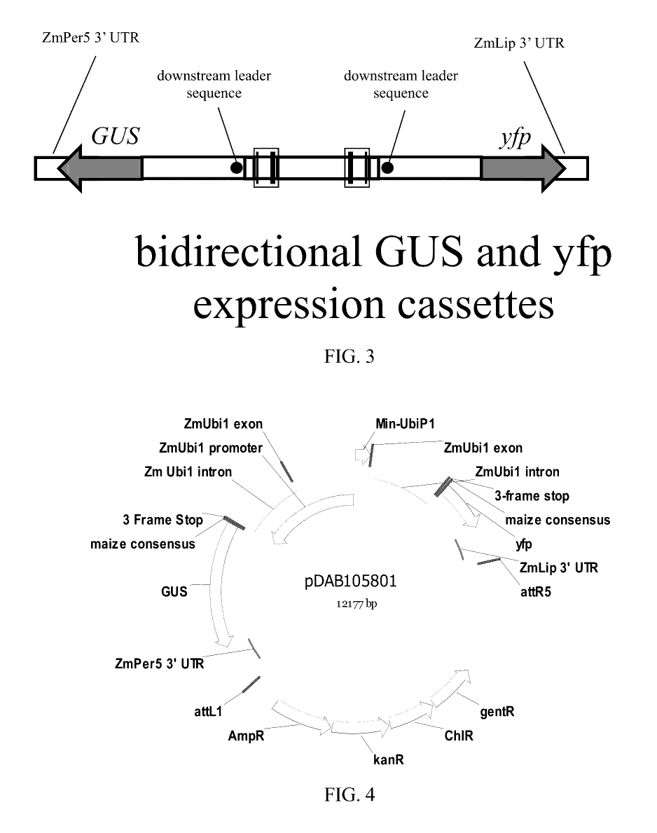

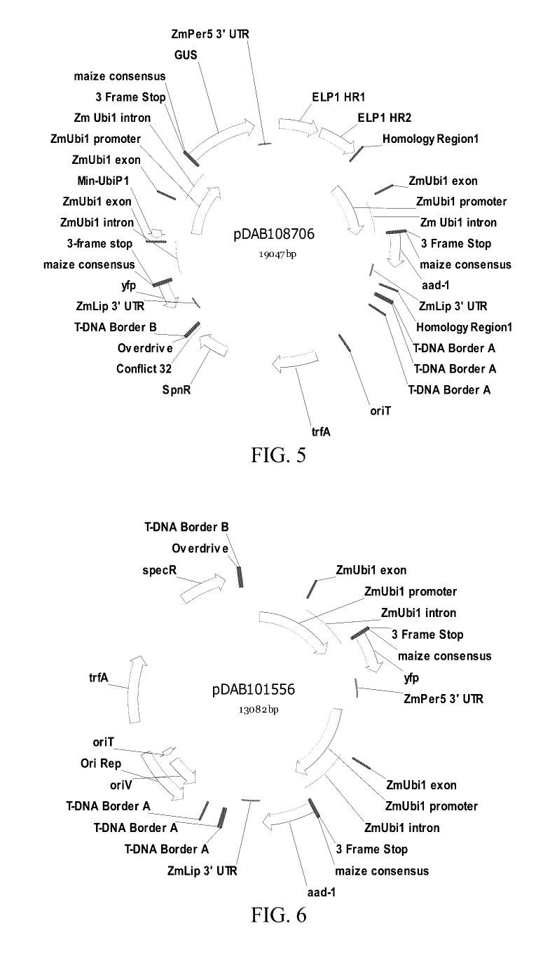

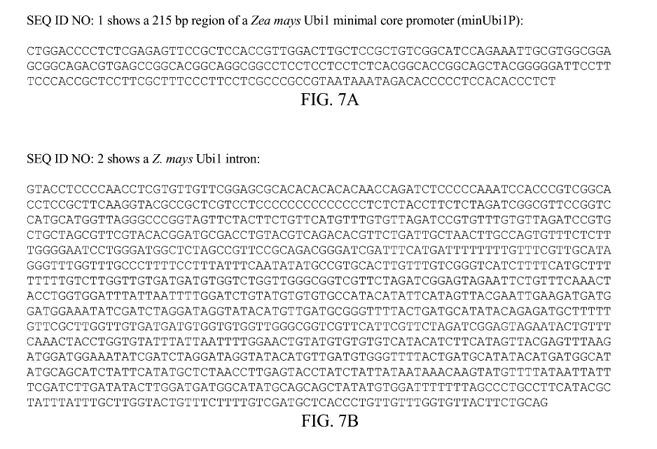

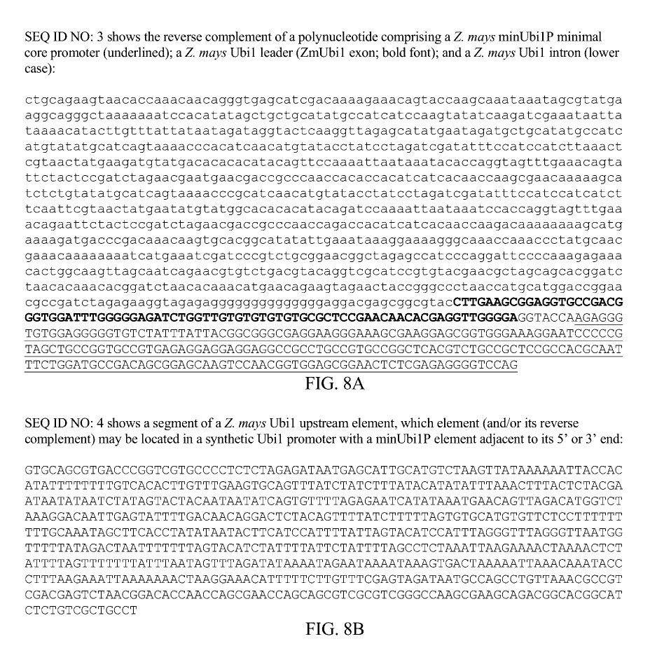

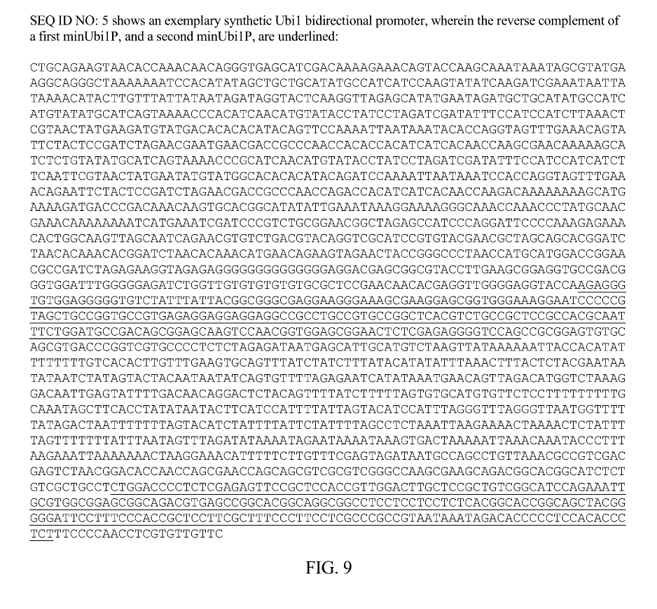

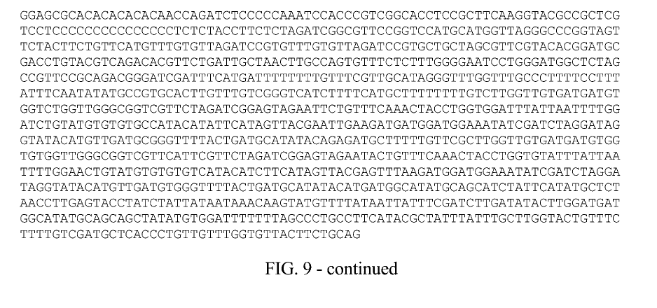

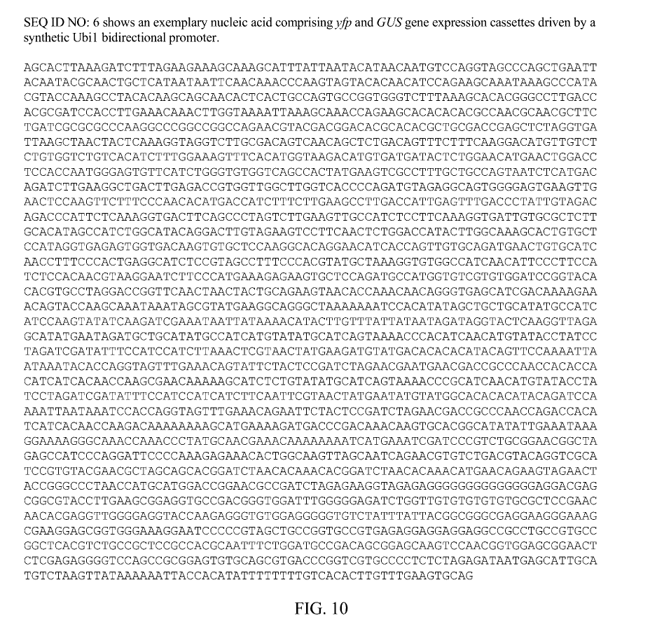

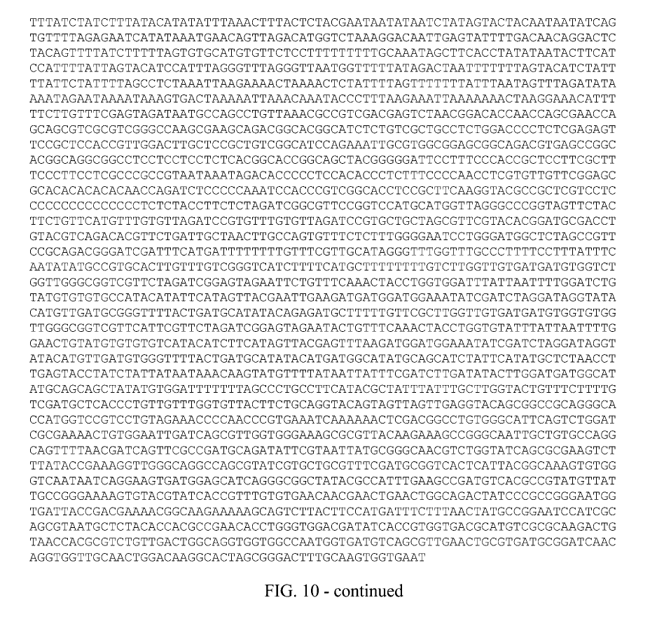

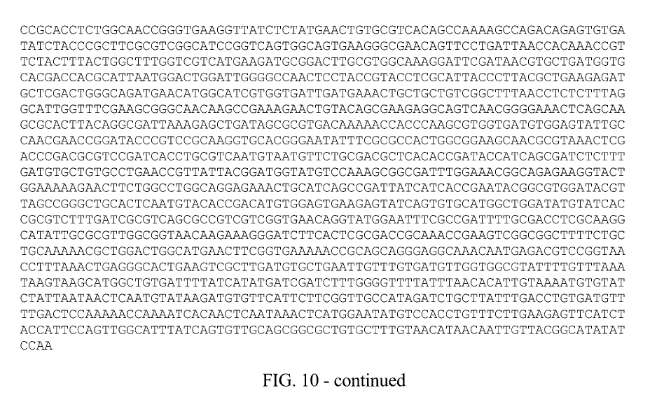

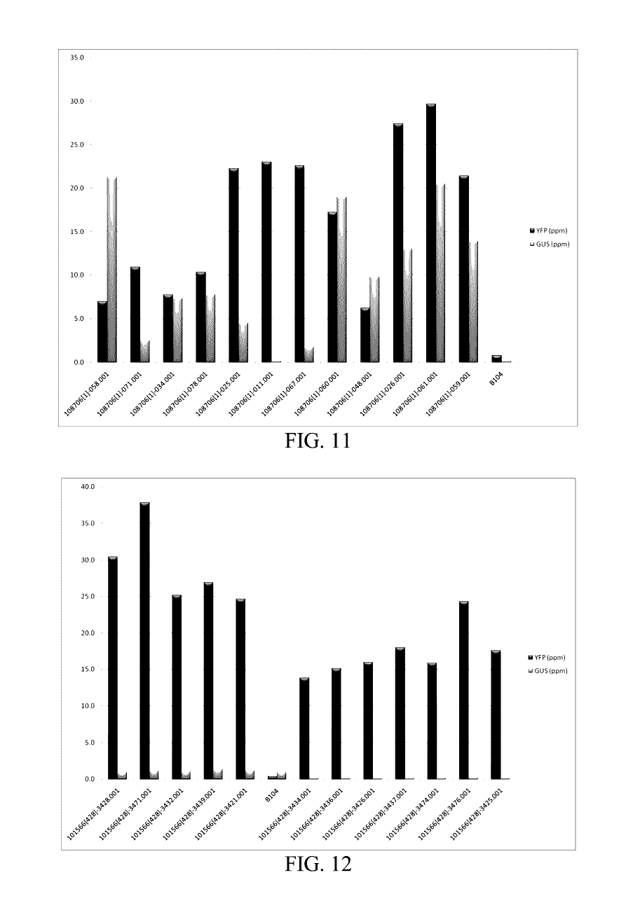

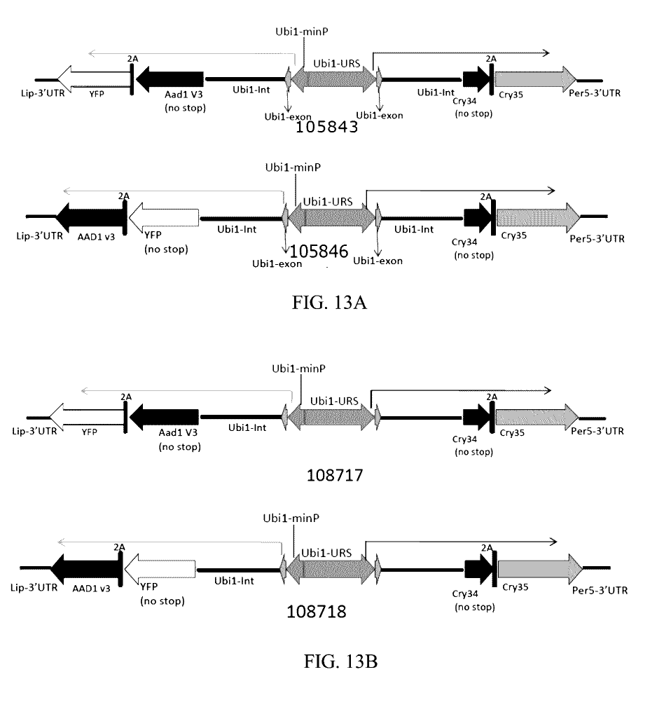

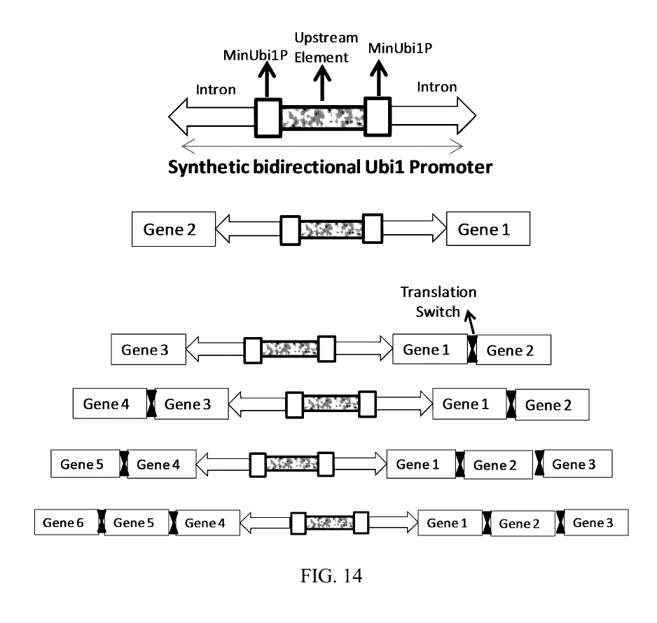

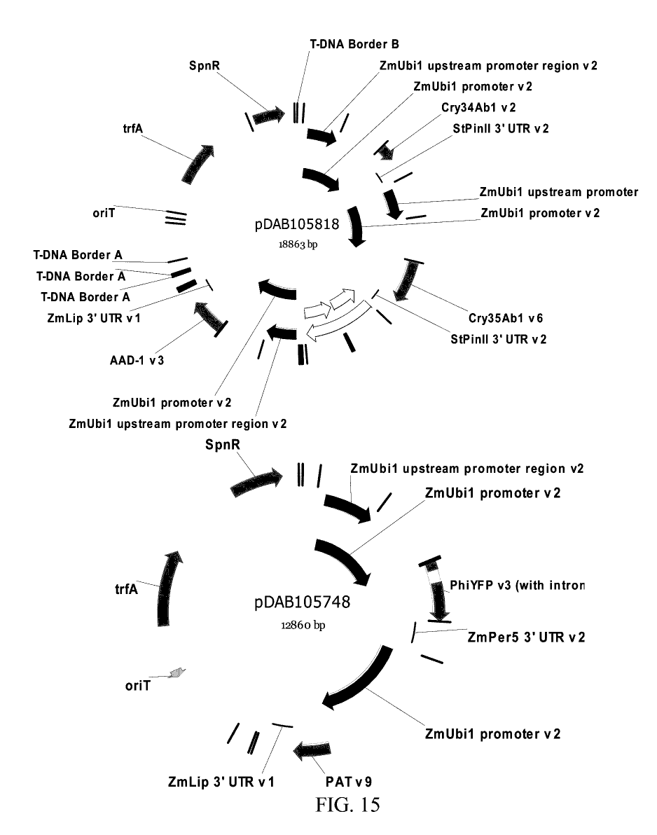

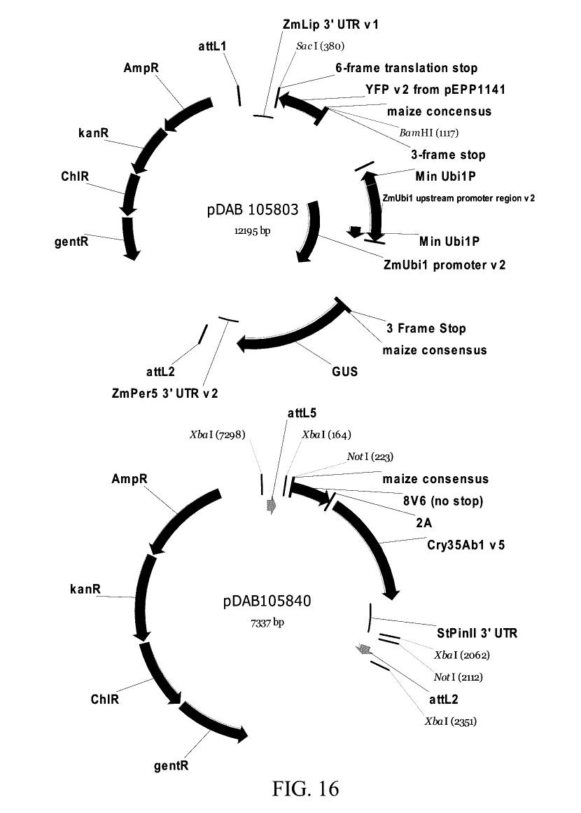

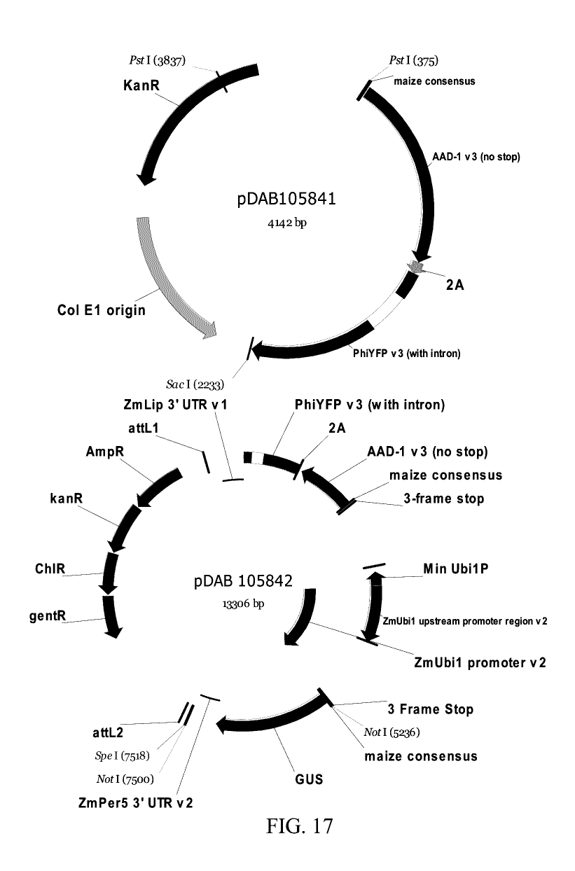

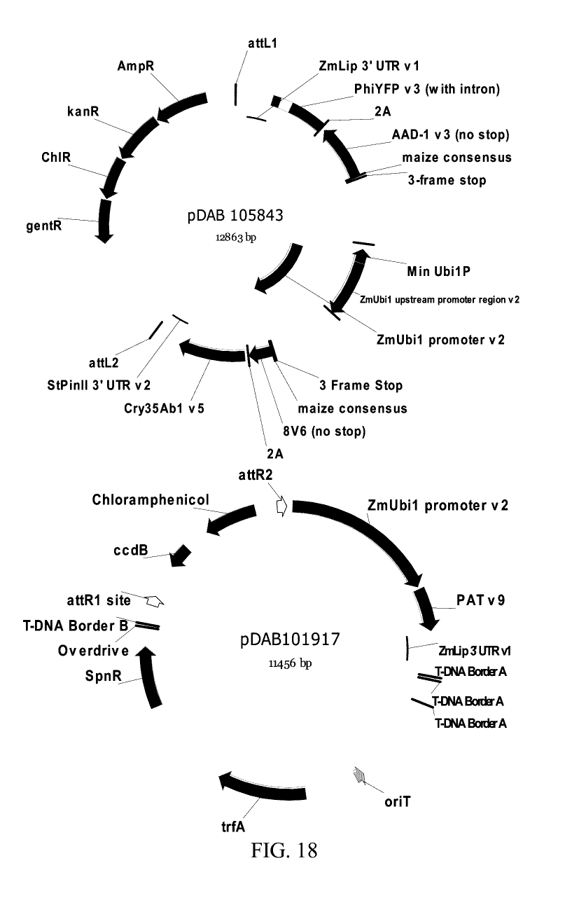

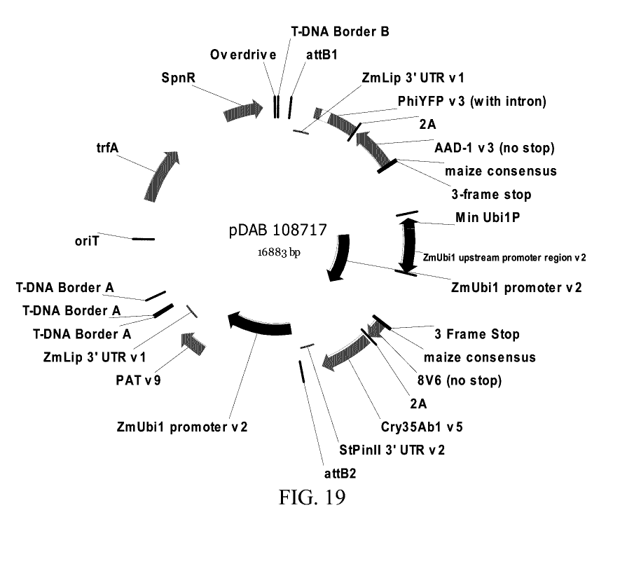

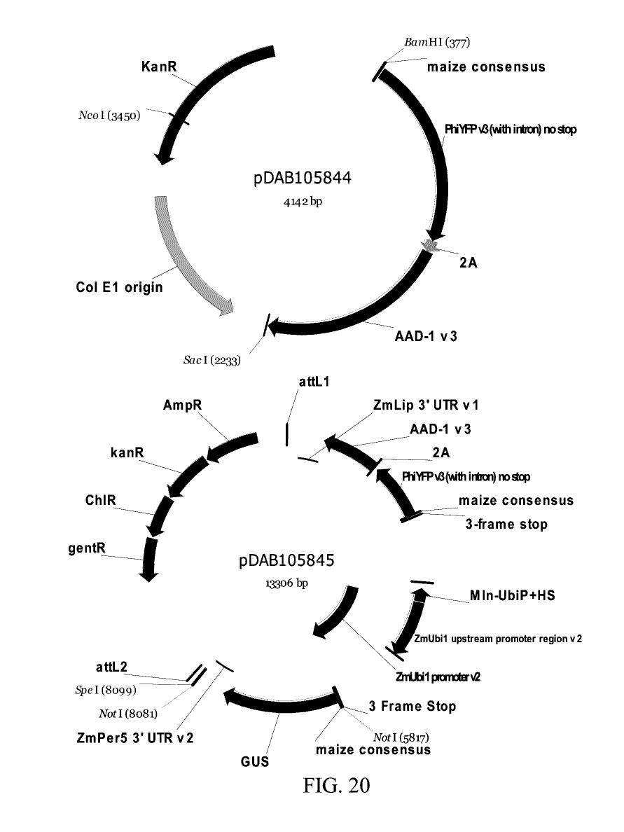

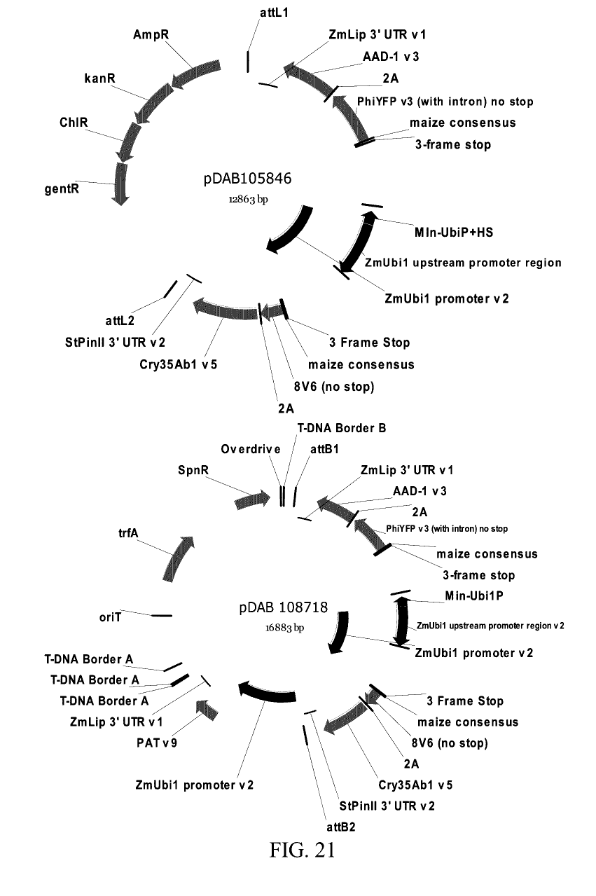

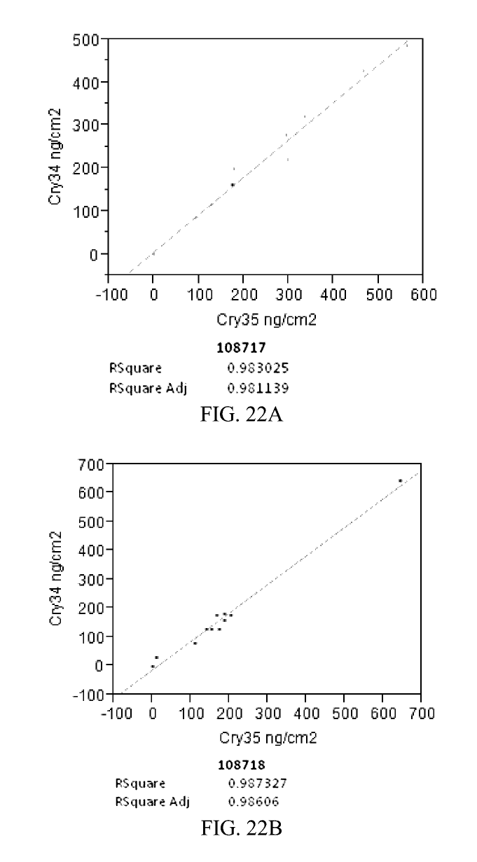

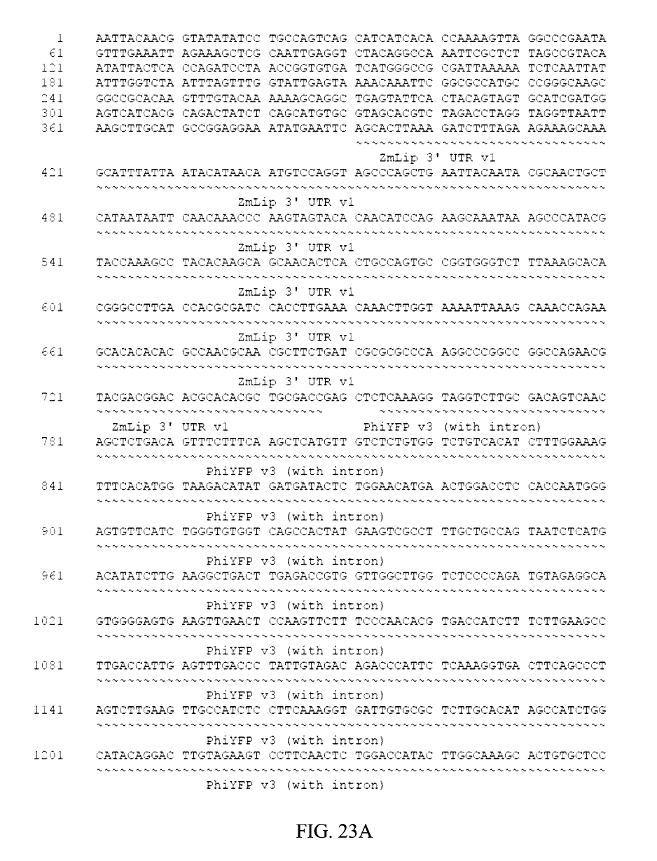

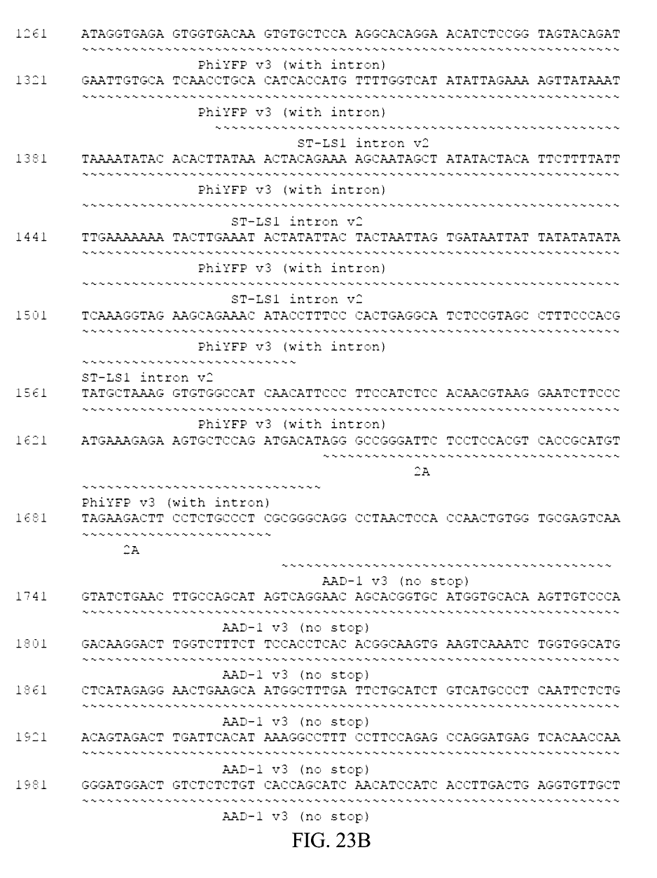

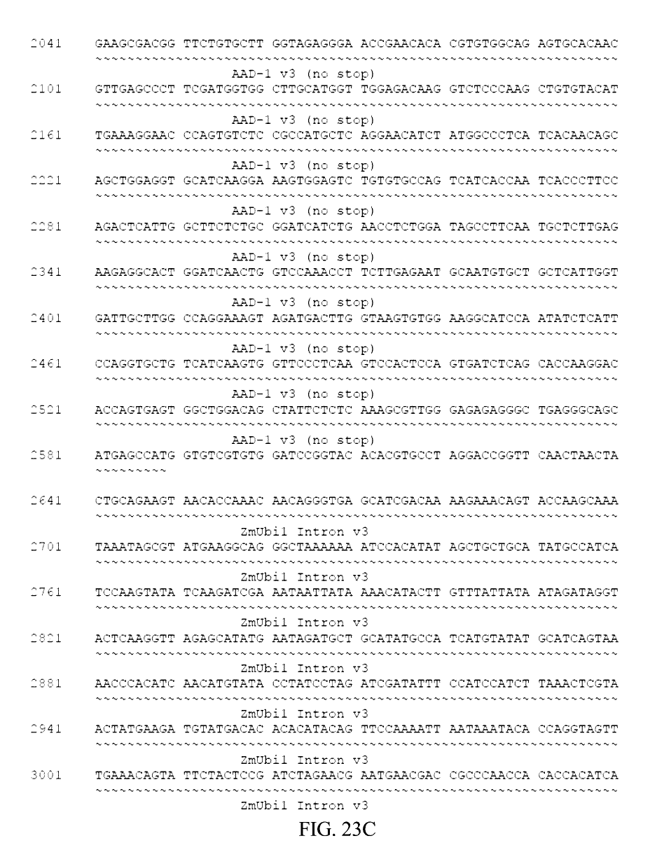

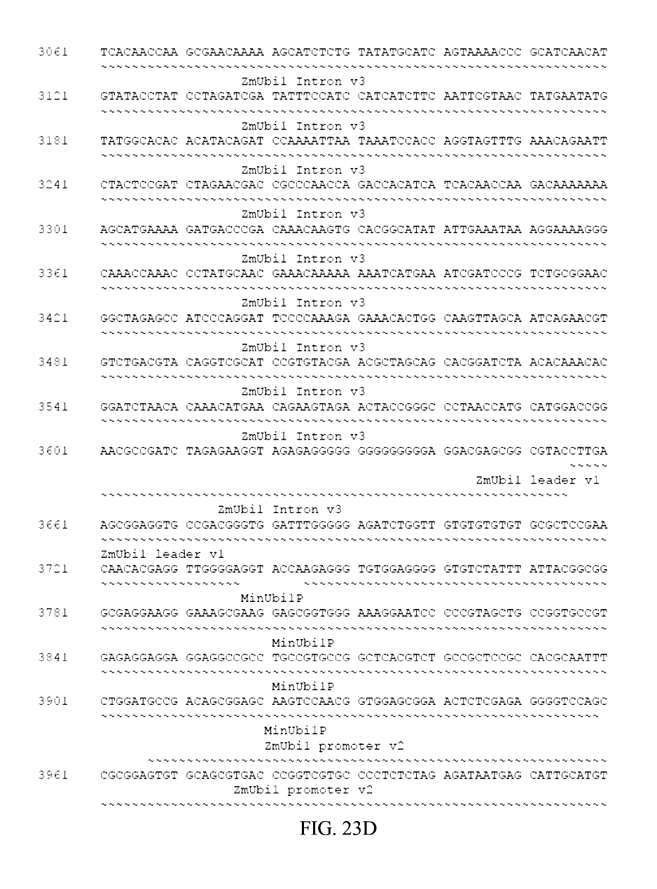

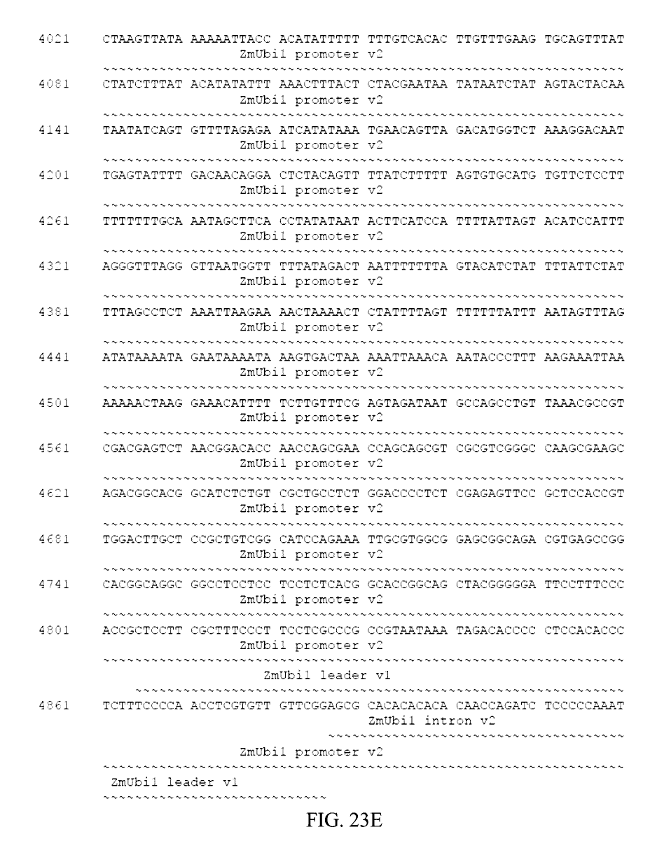

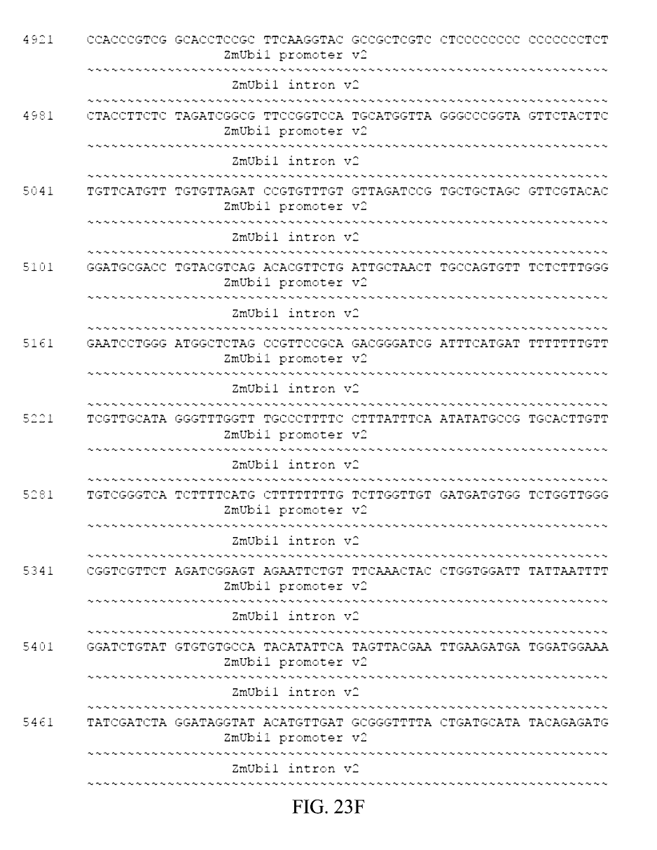

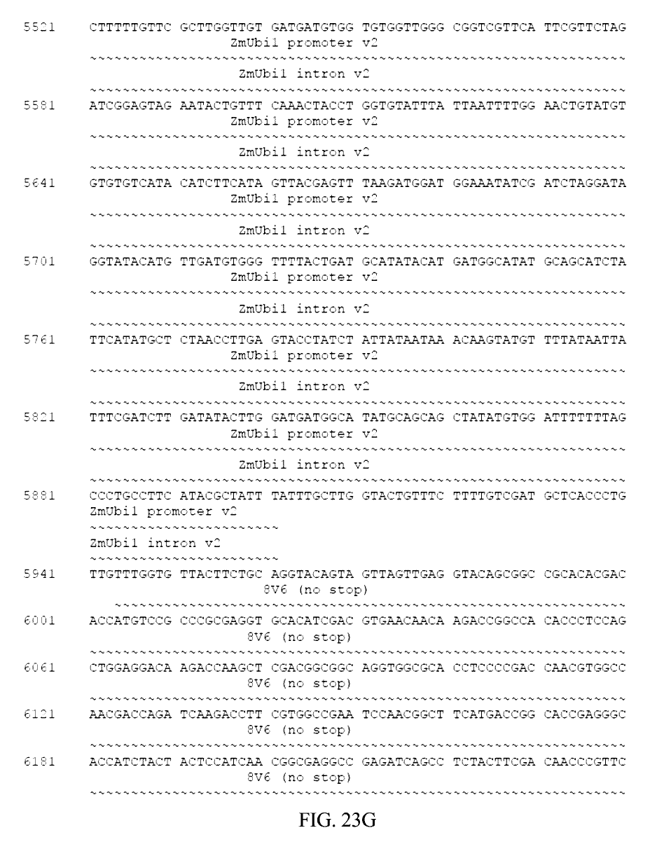

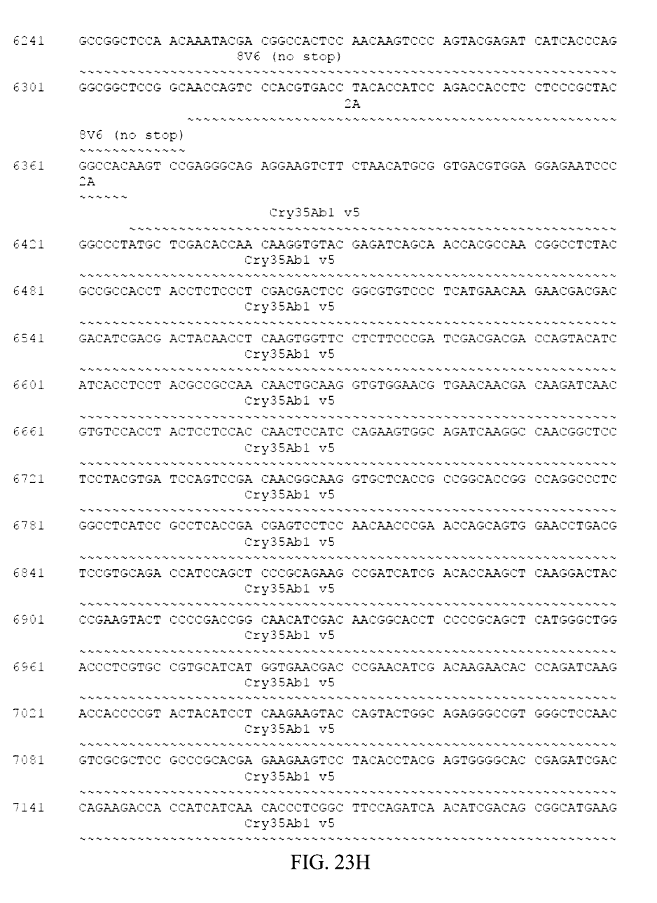

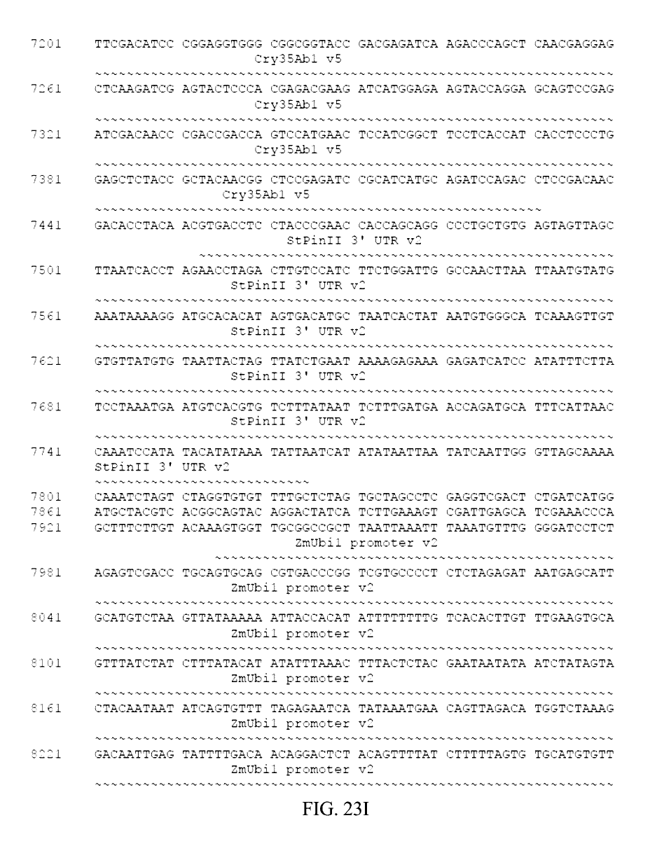

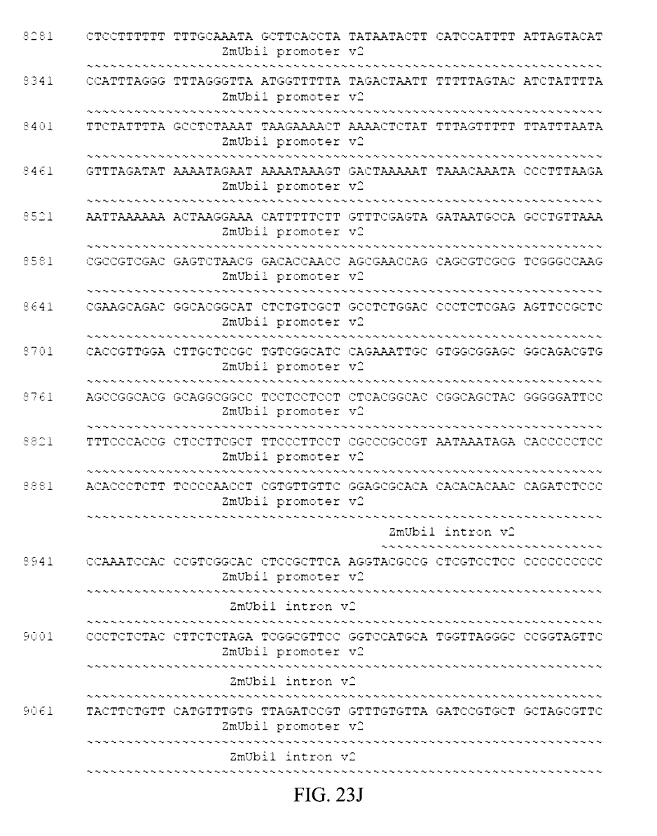

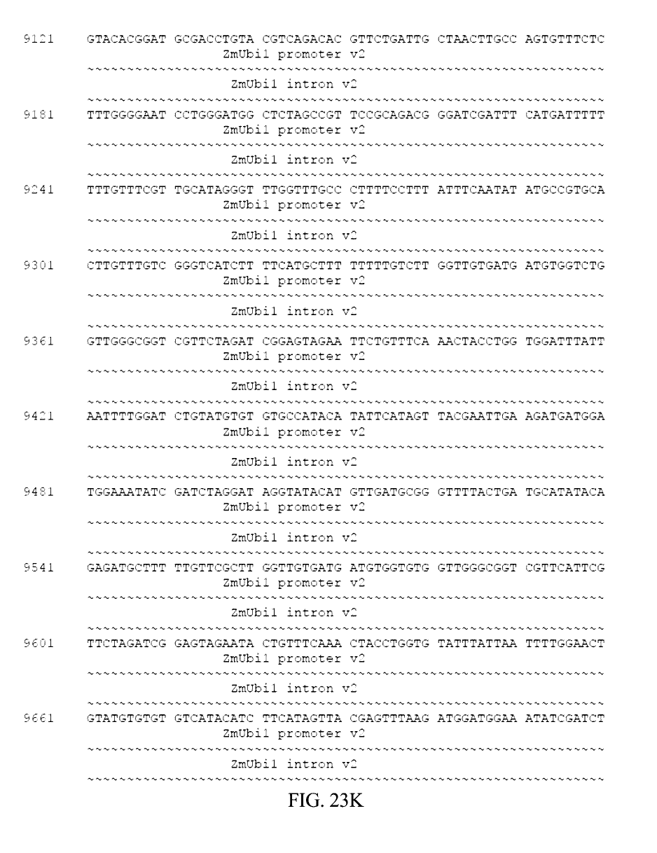

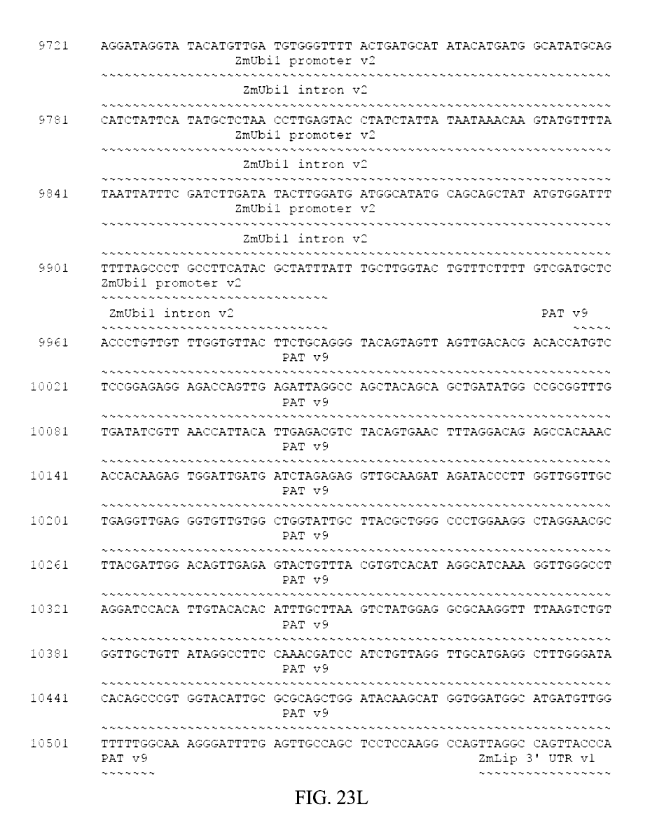

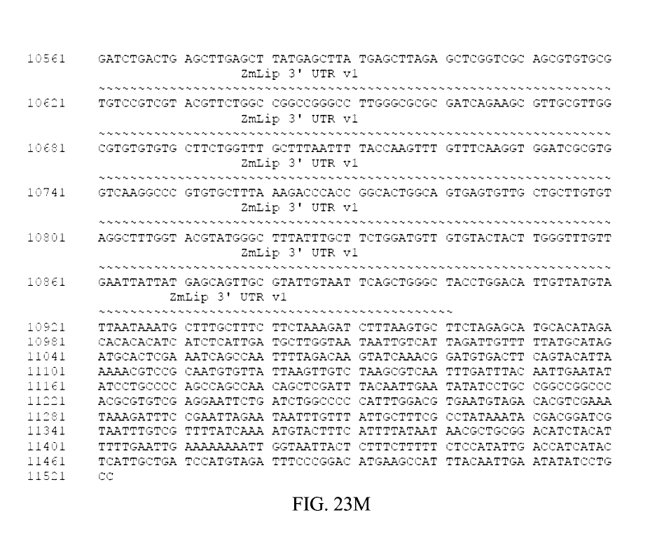

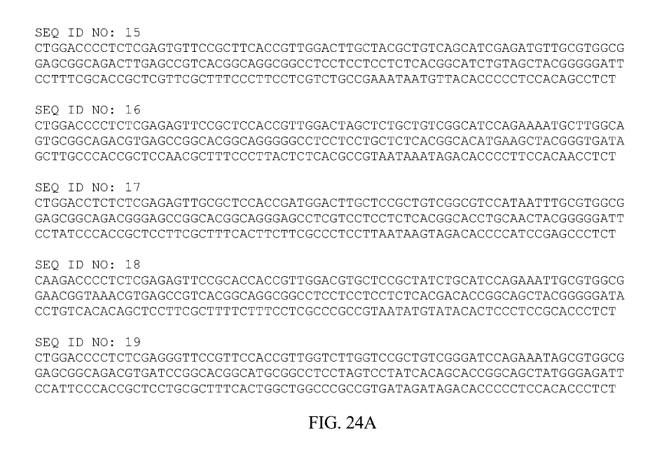

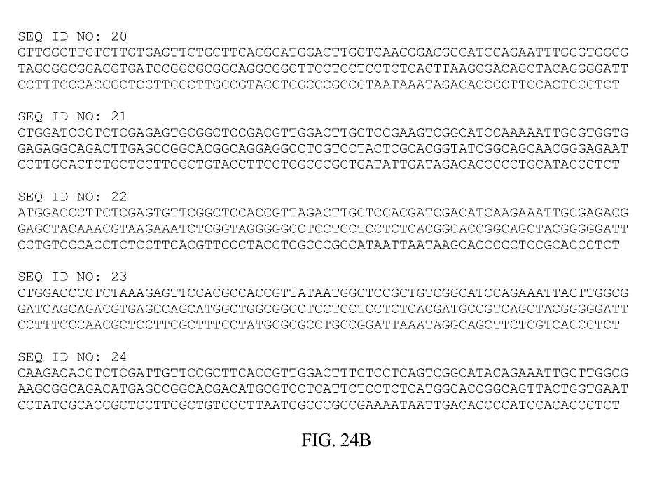

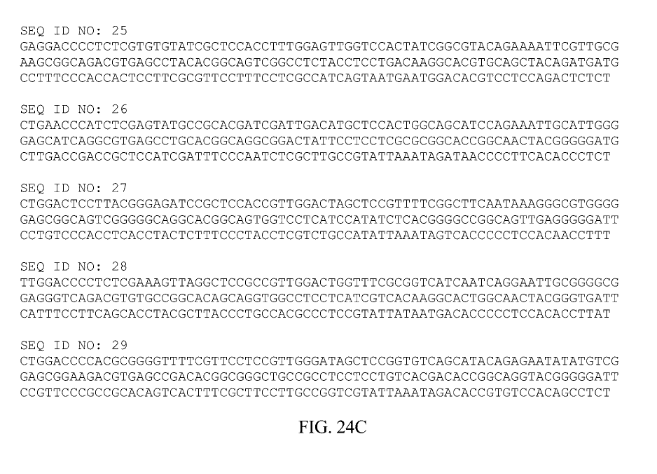

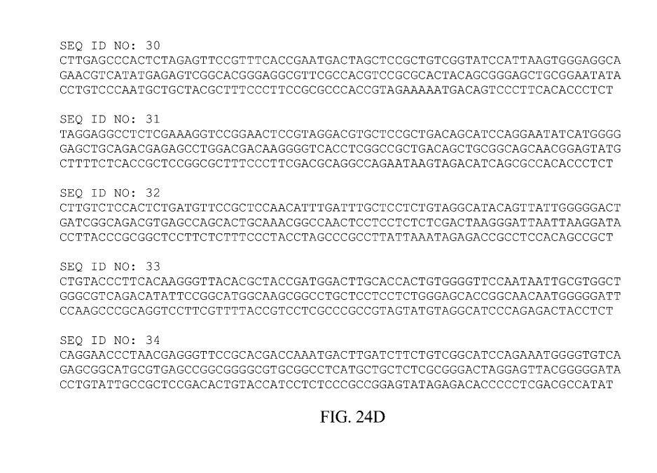

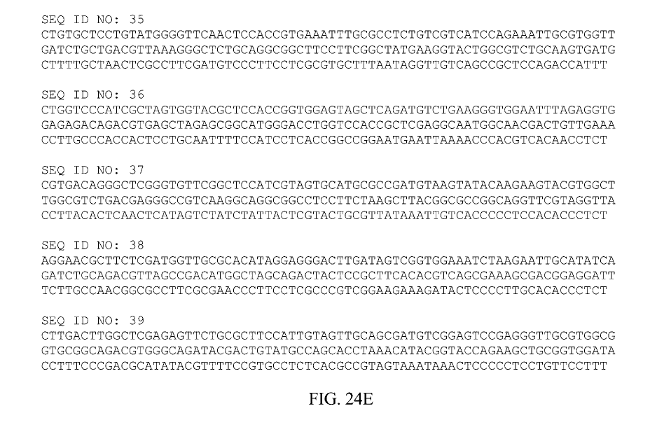

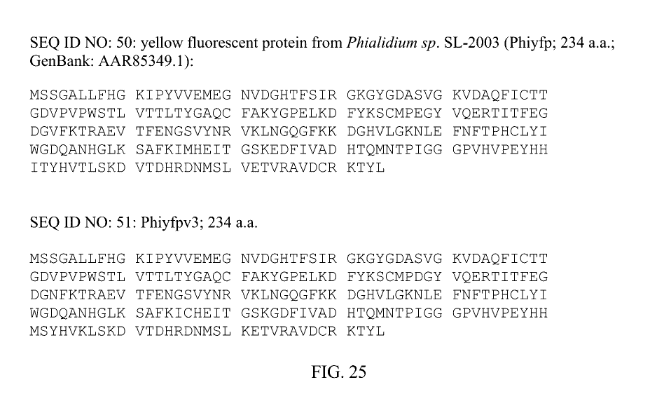

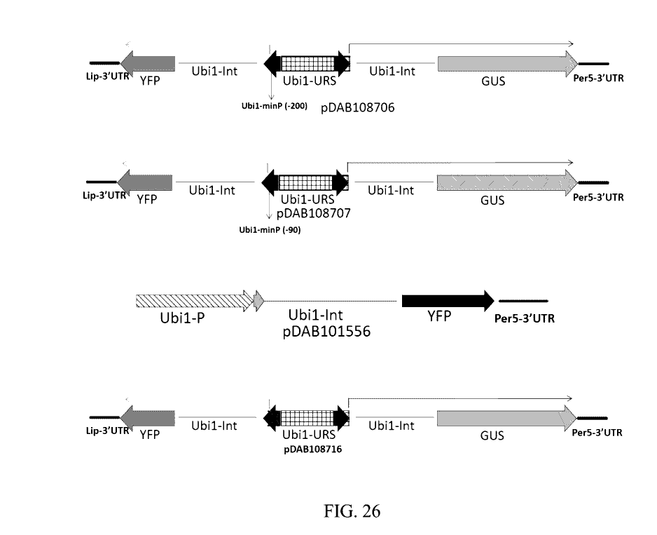

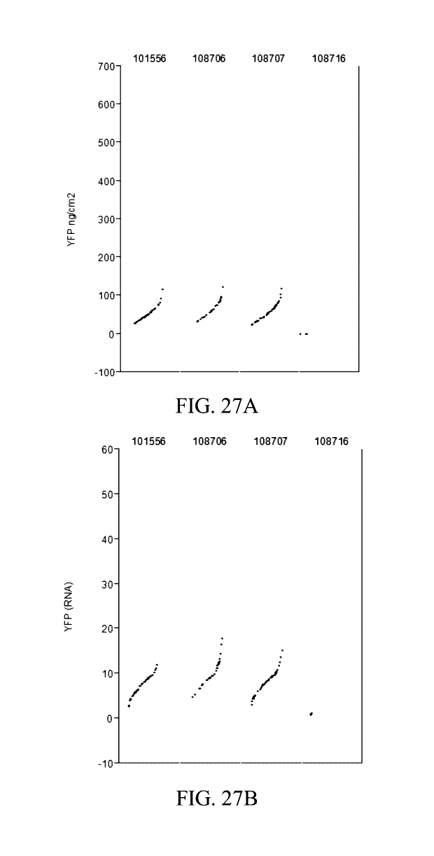

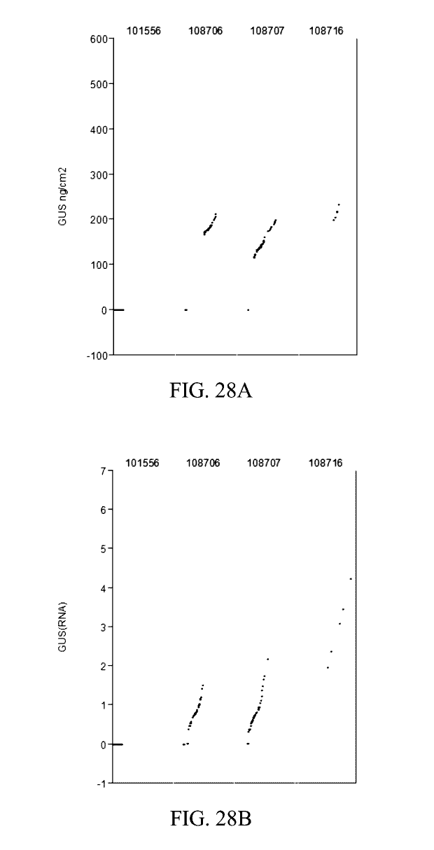

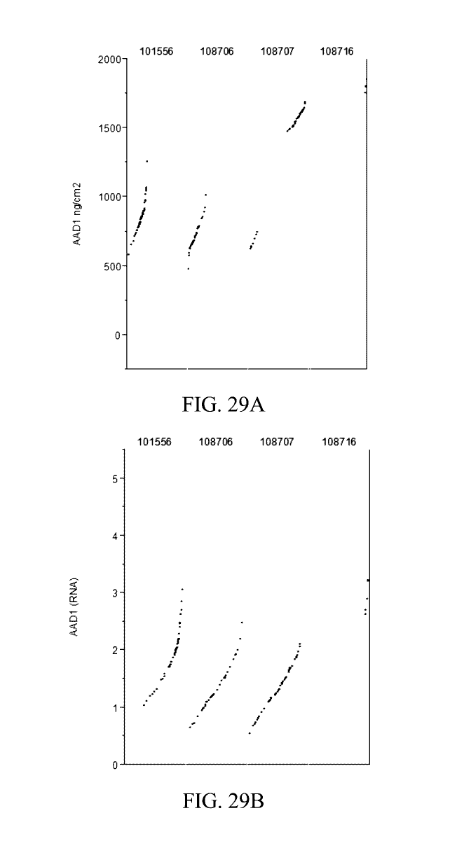

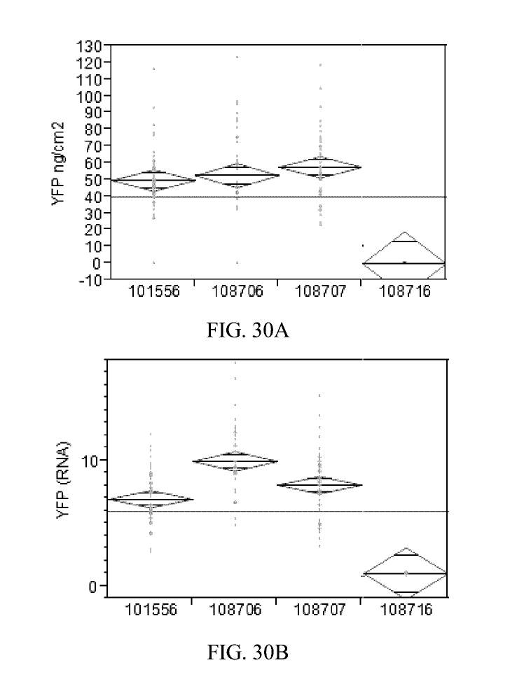

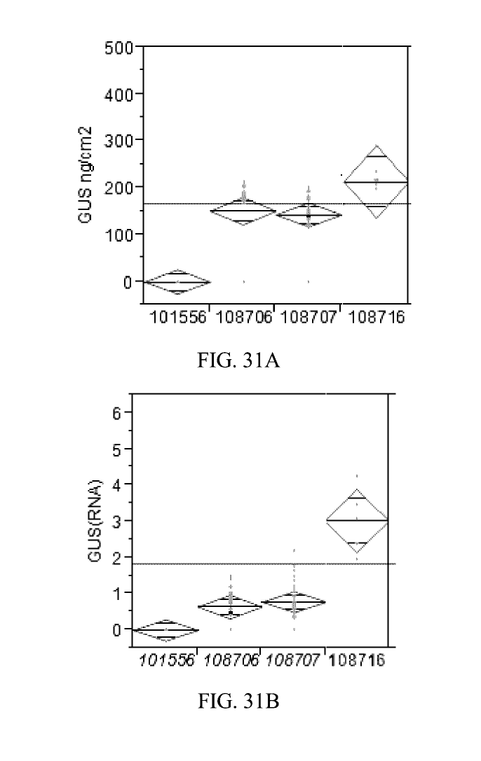

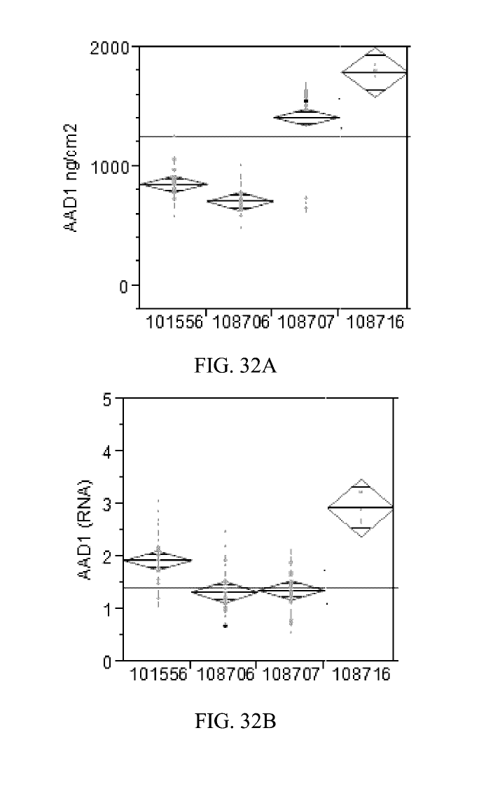

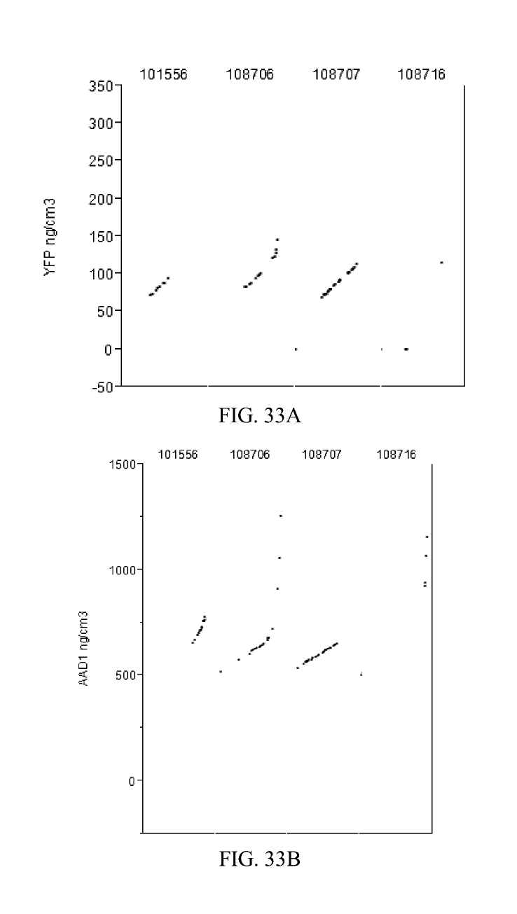

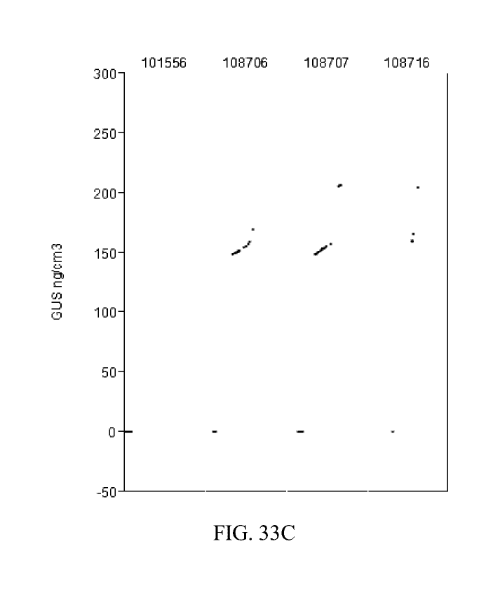

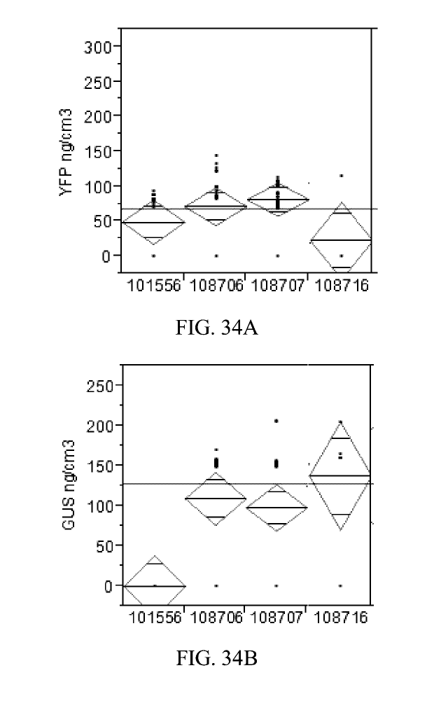

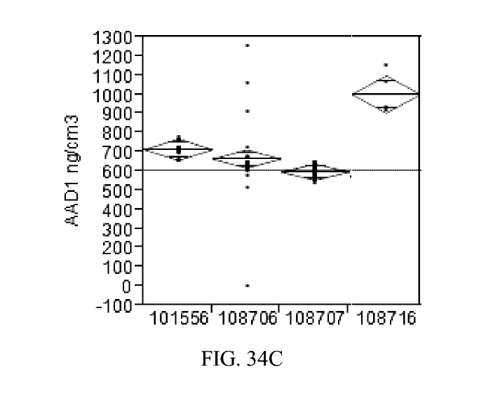

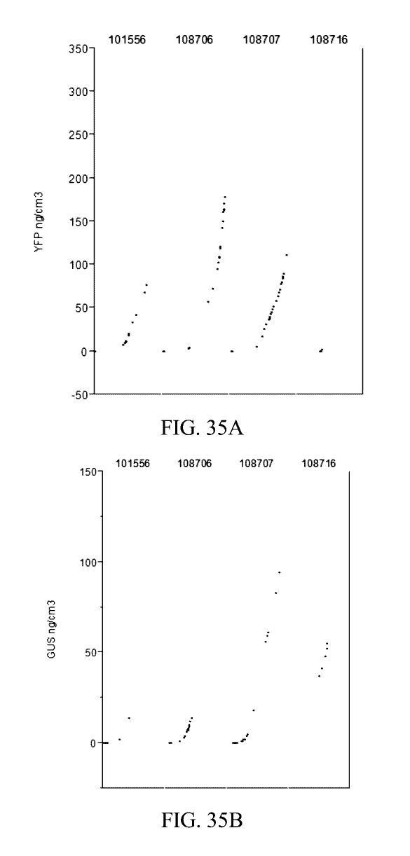

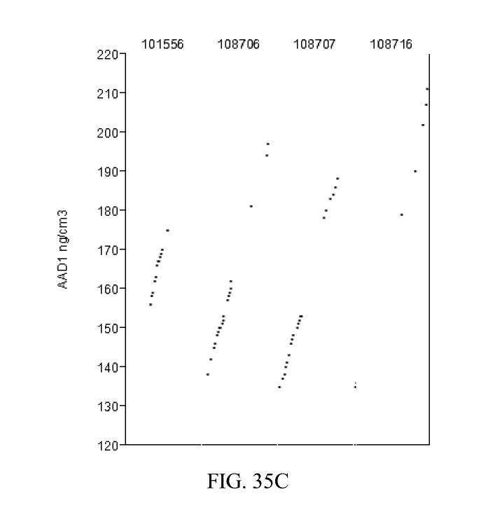

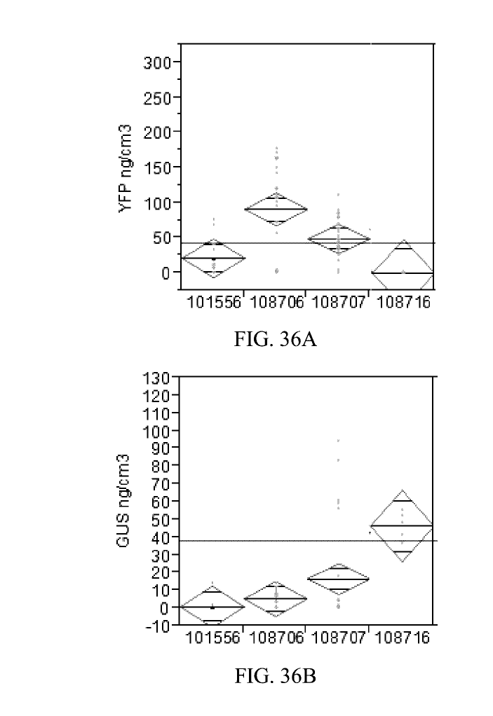

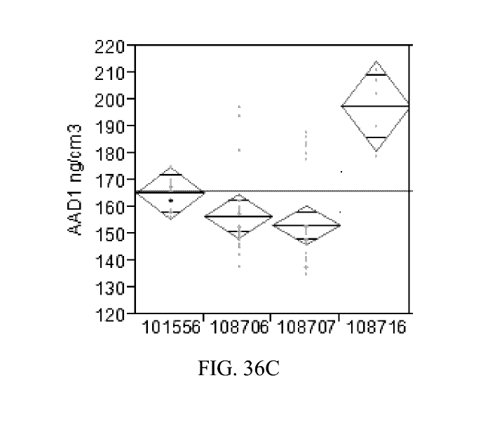

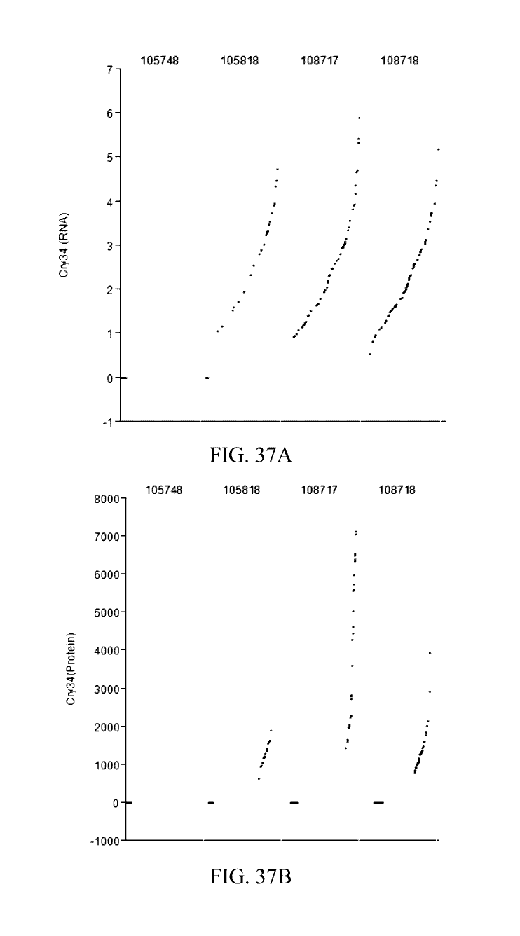

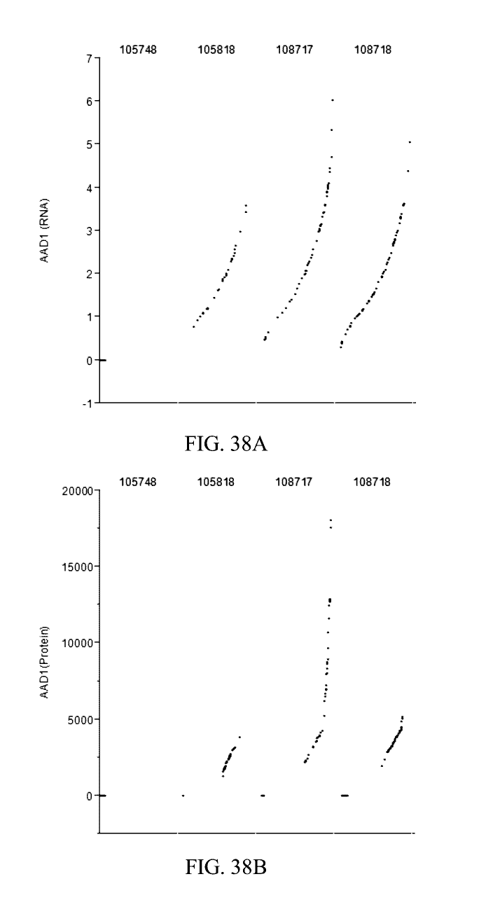

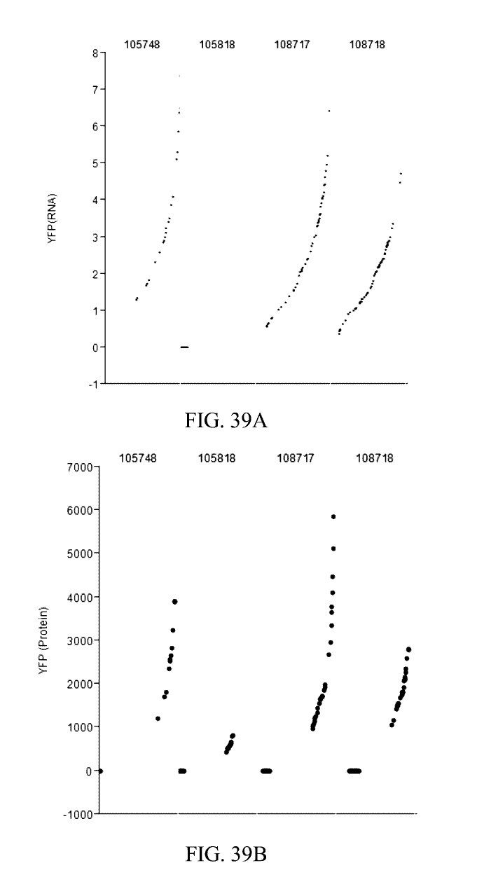

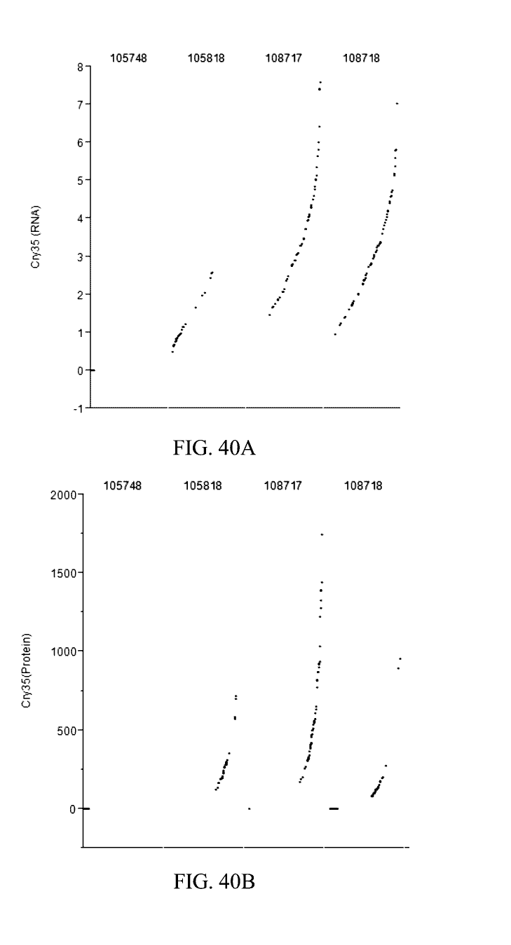

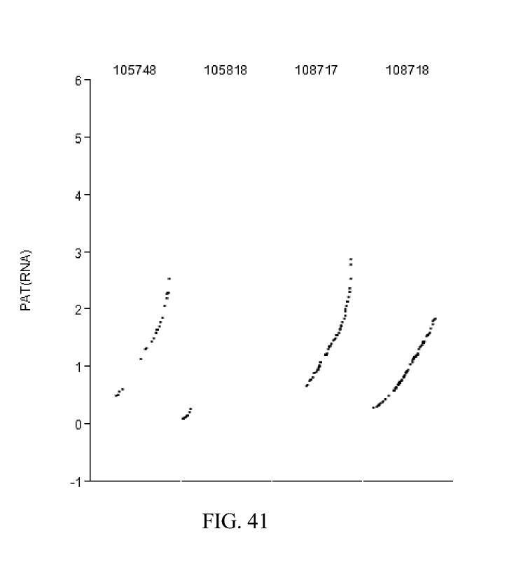

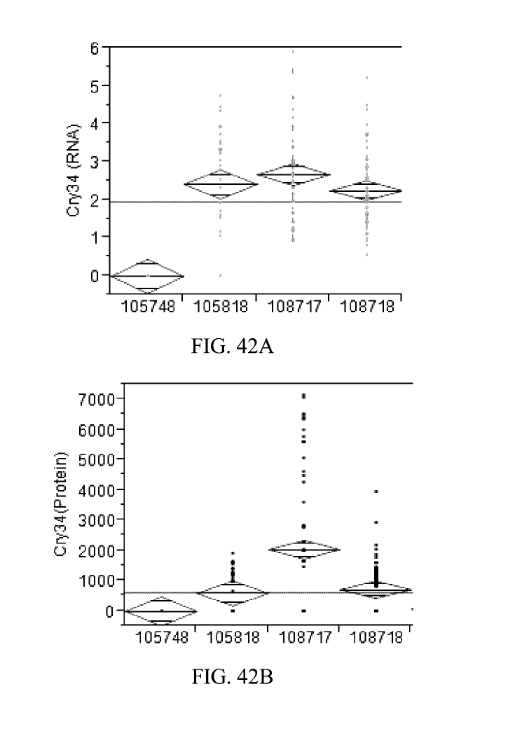

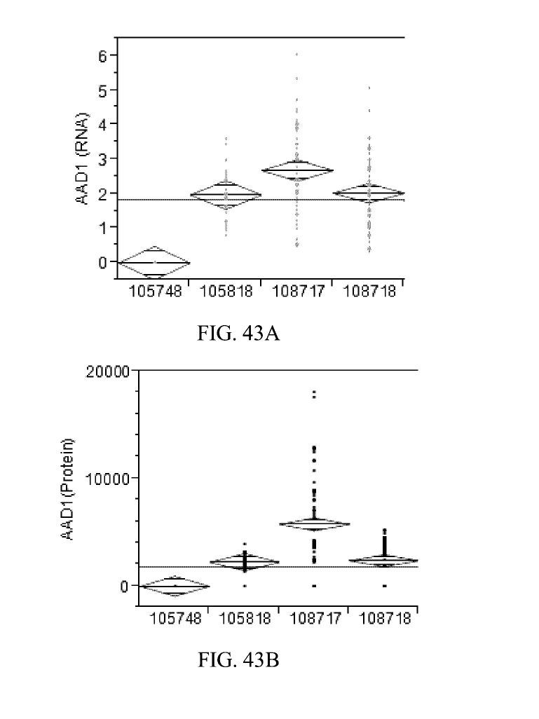

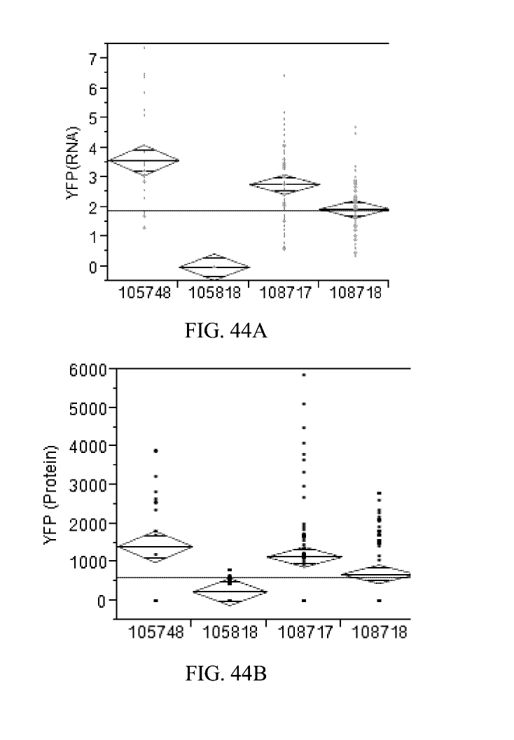

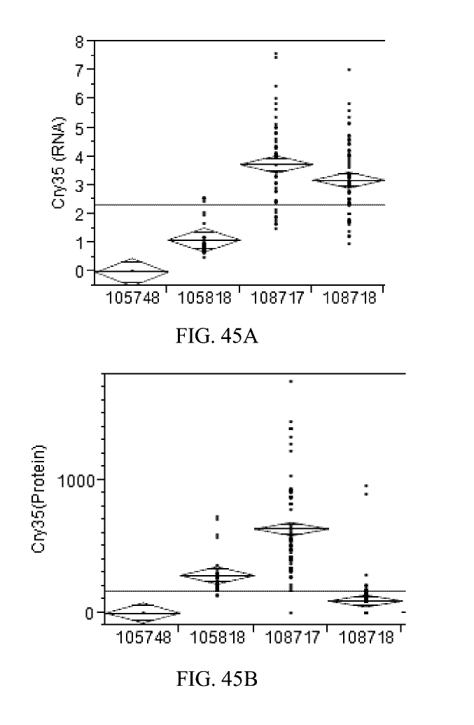

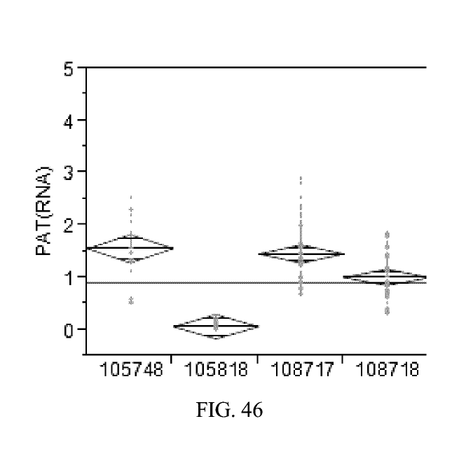

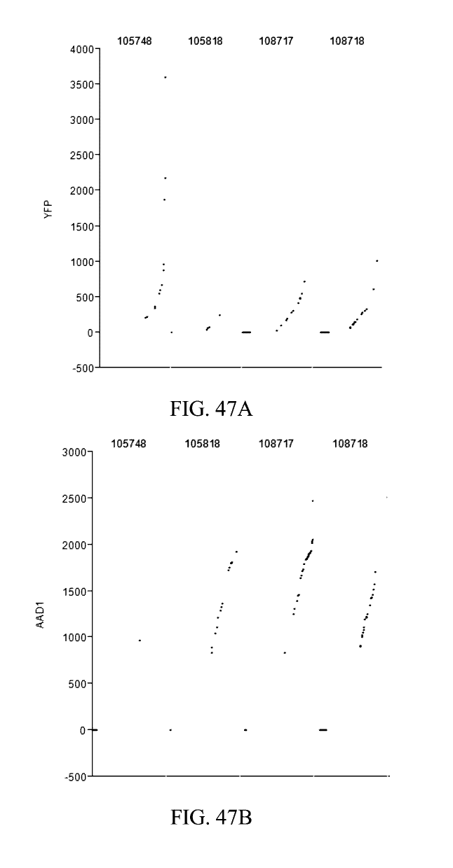

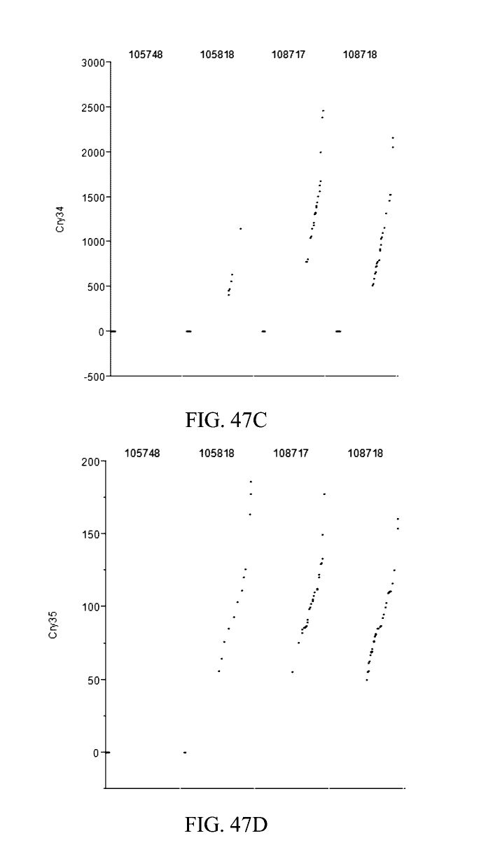

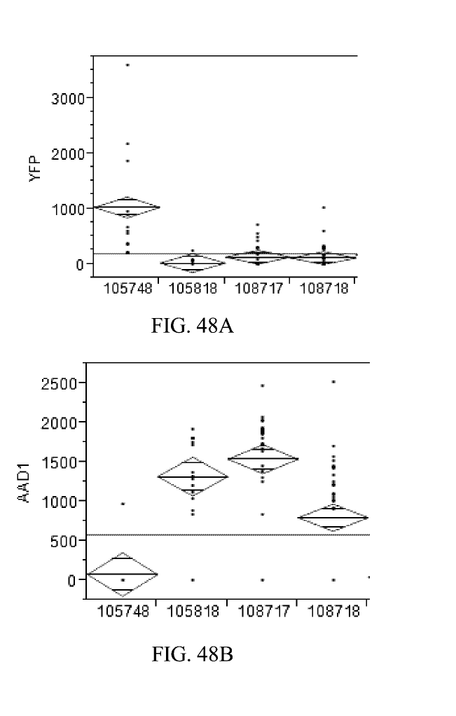

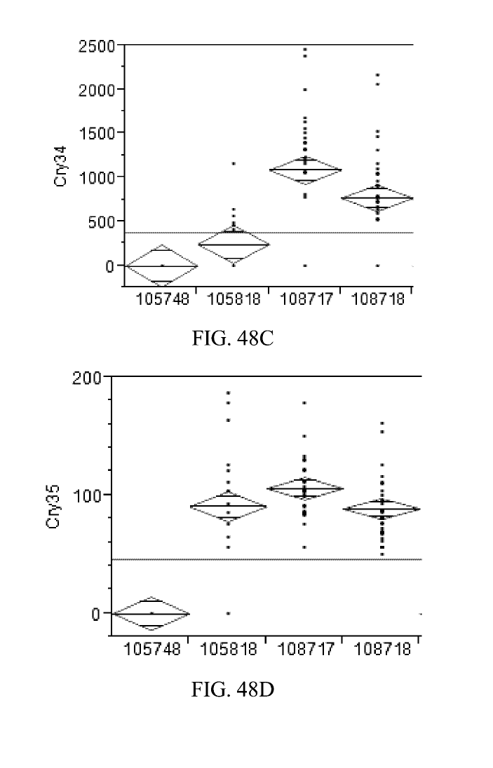

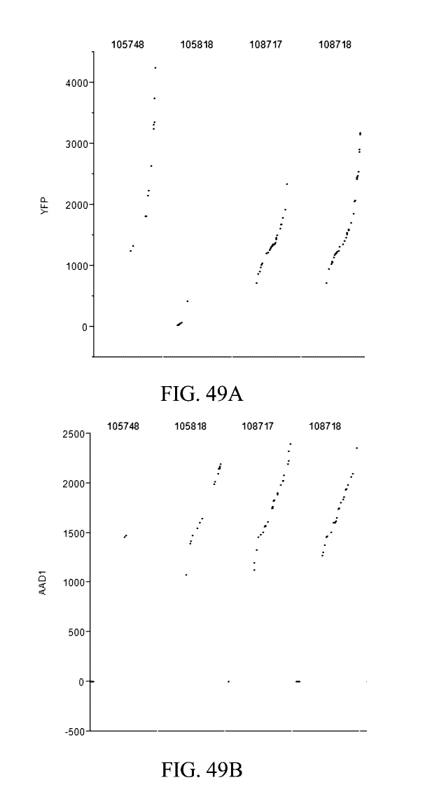

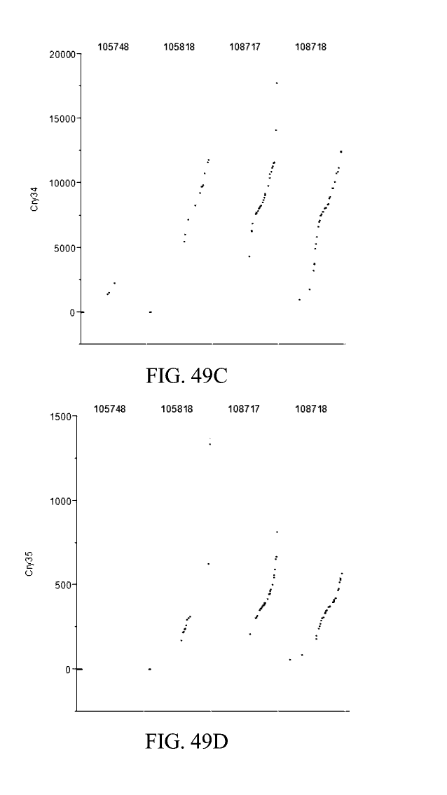

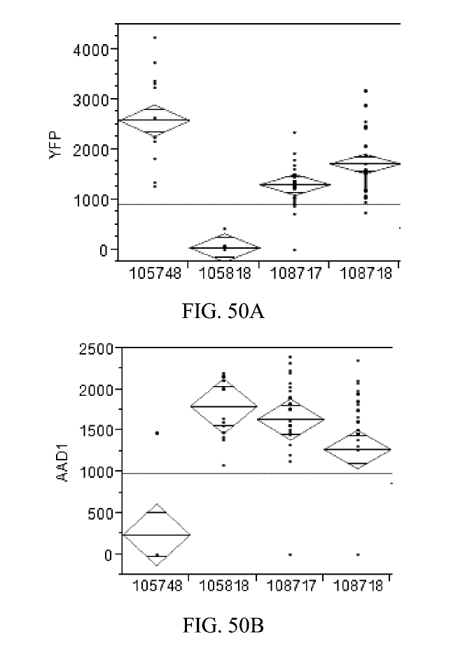

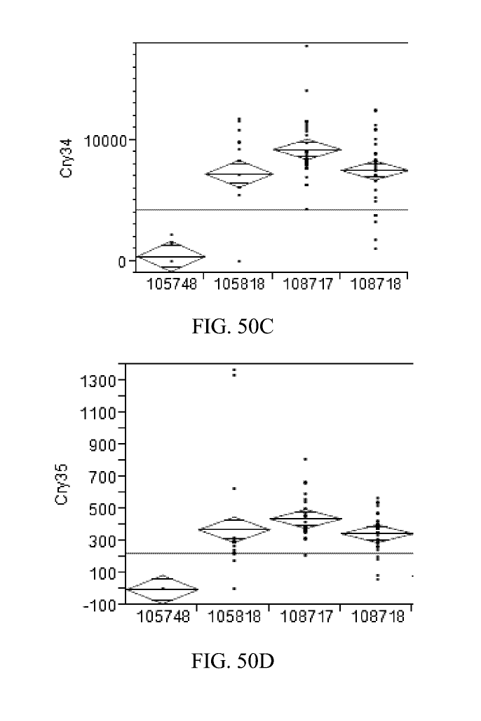

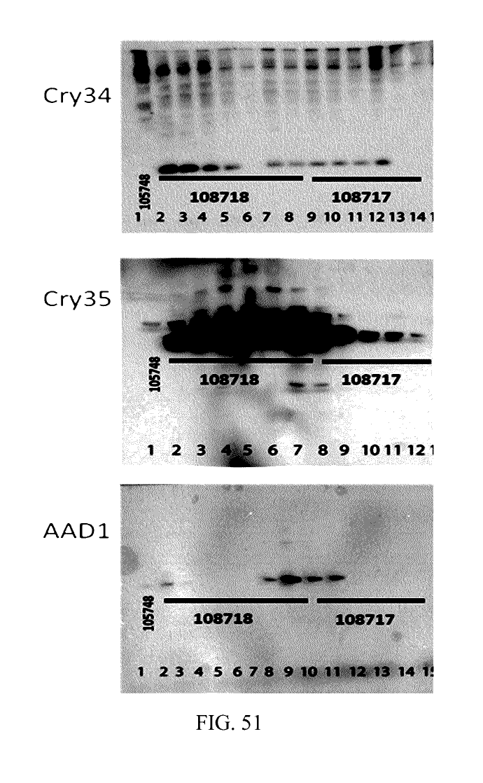

Reporter gene coding sequences are cloned downstream of each end of the synthetic bidirectional Ubi1 promoter. A yellow fluorescence protein (yfp) coding sequence is inserted downstream of the polynucleotide fragment which contains the minUbi1P, ZmUbi1 exon, and ZmUbi1 intron promoter elements. In addition, a downstream leader sequence containing a 3-frame stop polynucleotide sequence and the maize consensus polynucleotide (Kozak) sequence is added to the minUbi1P, ZmUbi1, exon and ZmUbi1 intron promoter elements fragment. AuidA (GUS) coding sequence is inserted downstream of the synthetic bidirectional Ubi1 promoter in reverse orientation with respect to the yfp sequence (SEQ ID NO: 6; see FIG. 3 for example). The resulting polynucleotide comprising the synthetic bidirectional Ubi1 promoter operably linked to the yfp and GUS genes is cloned into plasmid pDAB105801.

A binary vector which contained the GUS and yfp gene expression cassettes from plasmid pDAB105801 is completed via a GATEWAY™ CLONASE™ reaction (Invitrogen, Carlsbad, Calif.). The resulting vector, pDAB108706, contains the GUS, yfp, and aad1-1 gene expression cassettes within the T-strand region see FIG. 5 for example).

### Example 3

### Transient Expression of Genes Operably-Linked to a Synthetic Bidirectional Ubiquitin 1 Promoter

Representative examples of YFP and GUS transient expression in Zea mays embryos transformed with pDAB108706 are imaged. Both sides of the bidirectional ZmUbi1 promoter can drive robust expression of the operably linked yfp and GUS coding sequences. The YFP expression levels are comparable to the GUS expression levels. These observations confirm that both sides of the bidirectional ZmUbi1 promoter are biologically functional. Moreover, the minUbi1P element of the synthetic bidirectional Ubi1 promoter can express YFP at similar expression levels as compared to Zea mays callus transformed with a binary plasmid (pDAB101556) that contained a unidirectional ZmUbi1 promoter driving the yfp coding sequence. Expression of YFP or GUS is not detected in negative control immature embryos which are not transformed with a binary construct, and do not contain the yfp or GUS coding sequences.

### Example 4

### Stable Expression of Genes Operably-Linked to a Synthetic Bidirectional Ubiquitin 1 Promoter

Images of Zea mays callus cells that are stably transformed with the pDAB108706 binary vector, which contains the yfp coding sequence, can be observed. These cells are obtained from Z. mays embryos that have been propagating on selection 2 medium. The bidirectional ZmUbi1 promoter can drive robust expression of the yfp coding sequences. These results confirm that the Min-UbiP1 minimal promoter element of the bidirectional ZmUbi1 promoter is capable of expressing a reporter gene in stably-transformed Z. mays callus cells. The levels of expression of the YFP protein are similar as compared to YFP expression in Z. mays callus transformed with a control binary vector that contained the unidirectional ZmUbi1 promoter driving the yfP coding sequence (pDAB101556). Expression of YFP or GUS is not detected in the negative control callus that is not transformed with a binary construct and does not contain a yfp or GUS coding sequence.

### Example 5

### Transgene Copy Number Estimation Using Real Time TaqMan® PCR

Zea mays embryos are transformed with a binary vector containing a bidirectional ZmUbi1 promoter, pDAB108706, and other plants are transformed with a control binary vector, pDAB101556, The presence of yfp transgenes within the genome of both set of Z. mays plants is confirmed via a hydrolysis probe assay. Stably-transformed transgenic Z. mays plantlets that developed from the callus are obtained and analyzed to identify events that contained a low copy number (1-2 copies) of full-length T-strand inserts from the pDAB108706 binary vector and pDAB101556 control binary vector. Identified plantlets are advanced to the green house and grown.

The Roche Light Cycler480™ system is used to determine the transgene copy number for events that are transformed with the pDAB108706 binary vector, and for control events that are transformed with the pDAB101556 binary vector. The method utilizes a biplex TaqMan® reaction that employs oligonucleotides specific to the yfp gene and to the endogenous Z. mays reference gene, invertase (Genbank Accession No: U16123.1), in a single assay. Copy number and zygosity are determined by measuring the intensity of yfp-specific fluorescence, relative to the invertase-specific fluorescence, as compared to known copy number standards.

In Z. mays transformed with the pDAB108706 binary vector, a yfp gene-specific DNA fragment is amplified with one TaqMan® primer/probe set containing a probe labeled with FAM fluorescent dye, and invertase is amplified with a second TaqMan® primer/probe set containing a probe labeled with HEX fluorescence (Table 2). The PCR reaction mixture is prepared as set forth in Table 3, and the gene-specific DNA fragments are amplified according to the conditions set forth in Table 4. Copy number and zygosity of the samples are determined by measuring the relative intensity of fluorescence specific for the reporter gene, yfp, to fluorescence specific for the reference gene, invertase, as compared to known copy number standards.

TABLE 2
Forward and reverse nucleotide primer and fluorescent probes (synthesized by
Integrated DNA Technologies, Coralville, IA).

| Primer Name | SEQ ID NO: | Primer Sequence |
|---|---|---|
| YFP Forward Primer | SEQ ID NO: 7 | GATGCCTCAGTGGGAAAGG |
| YFP Reverse Primer | SEQ ID NO: 8 | CCATAGGTGAGAGTGGTGACAA |
| YFP Probe | — | ROCHE UPL Probe #125 CTTGGAGC |
| | | Cat# 04693604001 (Roche, Indianapolis, IN) |
| Invertase Forward | SEQ ID NO: 9 | TGGCGGACGACGACTTGT |
| Primer | | |
| Invertase Reverse | SEQ ID NO: 10 | AAAGTTTGGAGGCTGCCGT |
| Primer | | |
| Invertase Probe | SEQ ID NO: 11 | 5′HEX/CGAGCAGACCGCCGTGTACTACTACC/ |
| | | 3BHQ_1/3′ |
| AAD1 Forward Primer | SEQ ID NO: 12 | TGTTCGGTTCCCTCTACCAA |
| AAD1 Reverse Primer | SEQ ID NO: 13 | CAACATCCATCACCTTGACTGA |
| AAD1 Probe | SEQ ID NO: 14 | CACAGAACCGTCGCTTCAGCAACA |

Standards are created by diluting the vector, pDAB108706, into Z. mays B104 genomic DNA (gDNA) to obtain standards with a known relationship of pDAB108706:gDNA. For example, samples having one; two; and four copies of vector DNA per one copy of the Z. mays 13104 gDNA are prepared. One and two copy dilutions of the pDAB108706 mixed with the Z. mays B104 gDNA standard are validated against a control Z. mays event that is known to be hemizygous, and a control Z. mays event that is known to be homozygous (Z. mays event 278; see PCT International Patent Publication No. WO 2011/022469 A2). A TaqMan® biplex assay which utilizes oligonucleotides specific to the AAD1 gene and oligonucleotides specific to the endogenous Z. mays reference gene, invertase, is performed by amplifying and detecting a gene-specific DNA fragment for AAD1 with one TaqMan® primer/probe set containing a probe labeled with FAM fluorescent dye, and by amplifying and detecting a gene-specific DNA fragment for invertase with a second TaqMan® primer/probe set containing a probe labeled with HEX fluorescence (Table 2). The AAD1 TaqMan® reaction mixture is prepared as set forth in Table 3 and the specific fragments are amplified according to the conditions set forth in Table 4.

TABLE 3
Taqman ® PCR reaction mixture.

| Number of Reactions | μl each | Final Concentration |
|---|---|---|
| H₂O | 0.5 μL | — |
| PVP (10%) | 0.1 μL | 0.1% |
| ROCHE 2X Master Mix | 5 μL | 1X |
| Gene Forward Primer (10 μM) | 0.4 μL | 0.4 μM |
| Gene Reverse Primer (10 μM) | 0.4 μL | 0.4 μM |
| Gene Probe UPL#125 (5 μM) | 0.4 μL | 0.2 μM |
| Invertase Forward Primer (10 μM) | 0.4 μL | 0.4 μM |
| Invertase Reverse Primer (10 μM) | 0.4 μL | 0.4 μM |
| Invertase Probe (5 μM) | 0.4 μL | 0.2 μM |
| DNA Template | 2.0 μL | — |
| Total reaction volume | 10 μL | — |

The level of fluorescence that was generated for each reaction was analyzed using the Roche LightCycler 480™ Thermocycler according to the manufacturer's directions. The FAM fluorescent moiety was excited at an optical density of 465/510 nm, and the HEX fluorescent moiety was excited at an optical density of 533/580 nm. The copy number was determined by comparison of Target/Reference values for unknown samples (output by the LightCycler 480™) to Target/Reference values of four known copy number standards (Null, 1-Copy (hemi), 2-Copy (homo) and 4-Copy).

TABLE 4
Thermocycler conditions for PCR amplification.

| PCR Steps | Temp (° C.) | Time | No. of cycles |
|---|---|---|---|
| Step-1 | 95 | 10 minutes | 1 |
| Step-2 | 95 | 10 seconds | 40 |
| | 59 | 35 seconds | |
| | 72 | 1 second | |
| Step-3 | 40 | 10 seconds | 1 |

Results from the transgene copy number analysis of transgenic plants obtained via transformation with a bidirectional ZmUbi1 promoter construct (pDAB108706), and of transgenic plants obtained via transformation with a control unidirectional ZmUbi1 promoter YFP construct (pDAB101556) is shown in Table 5. Only plants with 1-2 copies of the yfp transgene were transferred to the greenhouse for further expression analyses.

TABLE 5
Transgene copy number estimation of the transgenic plants obtained
from bidirectional promoter construct and control construct.

| | Number of | | |
|---|---|---|---|
| | Embryos | Number of | |
| Construct | Transformed | Positive Events | 1-2 Copies of yfp |
| pDAB101566 | 100 | 31 | 13 |
| pDAB108706 | 110 | 29 | 12 |

### Example 6

### Whole Plant Stable Expression of Genes Operably-Linked to a Synthetic Bidirectional Ubiquitini Promoter

Whole plants that contained a low copy T-DNA number of the binary plasmid pDAB108706, and plants that contained a low copy number of the control binary plasmid pDAB101556, are grown in a greenhouse. Representative examples of stable expression of YFP in leaf and root tissue of transgenic T₀ maize plants obtained from Z. mays embryos transformed with pDAB108706 are analyzed. The bidirectional ZmUbi1 promoter can drive robust expression of the yfp coding sequences both in leaf tissues and root tissues. The microscopy analysis also confirms that the Min-UbiP1 minimal promoter element in the bidirectional ZmUbi1 promoter can drive a control binary plasmid (pDAB101556) that contains an unidirectional ZmUbi1 promoter driving expression of the yfP coding sequence. These control plants also show stable YFP expression in leaf tissues and root tissues.

### Example 7

### Western Blot Analysis of Genes Operably-Linked to a Synthetic Bidirectional Ubiquitini Promoter

Total Soluble Protein: Transformed T₀ maize plants were sampled at the V6 developmental stage. A total of four leaf punches from the youngest unfolded leaf were sampled into a matrix tube and placed into a matrix box. As a negative control, four leaf punches of two untransformed B104 maize plants at the V6 developmental stage were sampled into a matrix tube. A steel bead was placed into the matrix tubes with the samples, and then 400 μL PBST was added to each tube. The tubes were capped, and protein was extracted via bead beating at 1500 rpm for 5 minutes in a Kleco™ tissue grinder. Debris was pelleted via centrifugation.

A 5 μL sample from each tube was diluted to 25 μL with PBST in a 96-well microtiter plate. These samples were analyzed for total soluble protein using a BCA protein assay kit (Thermo Scientific Pierce, Rockford, Ill.) according to the manufacturer's directions. Bovine senim albumin (BSA) standards provided in the kit were analyzed in duplicate, and the average of the values was used to generate a standard curve that was subsequently used to calculate total soluble protein for each sample. The total soluble protein for each sample was then normalized to mg/μL.

TABLE 6
Western blot protocol.

| Step | Condition | Time | |
|---|---|---|---|
| First Wash | PBST | 5 | min. |
| Primary | 2 μg/mL rabbit anti-PhiYFP (Axxora, | 60 | min. |
| Hybridization | San Diego, CA) in StartingBlock ™ T20 | | |
| | (Thermo Fisher Scientific Inc., | | |
| | Waltham, MA) | | |
| Rinse | PBST | 3 × 5 | min. |
| Secondary | horse radish peroxidase (HRP)-conjugated | 30 | min. |
| Hybridization | goat anti-rabbit IgG | | |
| Second Wash | PBST | 3 × 5 | min. |
| Rinse | PBS | 3 × 2 | min. |

YFP/Phiyfp Western Blot Analysis: In the 96-well microtiter plate, each 5 μL sample of extracted protein is diluted with 5 μL 2× Laemmli Buffer +2-β-mercaptoethanol. Control samples of purified YFP/Phiyfp in HEPES buffer (50 mM HEPES, 200 mM KCl, 10% glycerol) is purchased from Axxora (San Diego, Calif.). The samples are prepared in the same plate by diluting 1:1 with Laemmli buffer to produce a standard curve of the following concentrations: 0.5 ng/μL, 0.25 ng/μL, and 0.125 ng/μL. Samples are heated in a Thermocycler at 95° C. for 30 minutes, and then cooled to 4° C. A Bio-Rad Criterion gel™ is then assembled using MES/SDS buffer. The samples are allowed to warm to room temperature, and 10 μL of sample are loaded into each well of two gels. In addition, samples of purified YFP/Phiyfp used for a standard curve, and protein ladder marker, are loaded into wells of the gel. The gels are electrophoretically run at 150 V and 150 mA for 90 min. After the run, the gel casings are opened and the proteins are transferred to a nitrocellulose membrane using the iBlot System™ (Invitrogen). Protein is transferred from the gel to the membrane by running a current of 20 V for 10 minutes. The nitrocellulose membrane is removed and placed in StartingBlock T20™ blocking buffer overnight at 4° C. The blocking buffer is then discarded, and the membrane is processed using the protocol set forth in Table 6.

Antibody binding is detected using the Amersham ECL™ plus chemiluminescent detection system following the manufacturer's directions. Film is exposed at 10 minutes and 30 minutes. The 10 minute exposed film is used to quantify protein, and the 30 minute overexposure film is used to confirm the absence of protein in B104 and other control samples. The membrane is taped to the back of the exposed film, and protein is quantified via pixel density analysis. The pixel density of the purified protein standards is first used to generate a standard curve that is used to quantify protein in the samples. Though membrane shows bands for a PhiYFP monomer and dimer even in the purified standard, only the PhiYFP monomer is used to quantify protein expression. Values for the protein are then normalized to ng/μL. The ratio of normalized total soluble protein (TSP) to PhiYFP is calculated to the units of ng YIP/mg TSP, or alternatively, parts per million (ppm).

GUS Western Blot Analysis: Expression of GUS protein is quantified in a similar manner to PhiYFP, with the following exception: a 10 μL sample of extract is diluted 1:1 with 2× Laemmli 2-β-mercaptoethanol, denatured at 95° C. for 30 minutes, and then 15 μL are loaded into the gel. Processed membranes with film (1 minute exposure) are overlayed with the membrane for pixel density analysis.

Results of a Western blot analysis of 12 transgenic T₀ maize plants obtained from Z. mays embryos transformed with the binary vector, pDAB108706, is shown in FIG. 11. The bidirectional ZmUbi1 promoter shows robust expression of the yfp and GUS coding sequences from leaf tissue. These observations confirm that the Min-UbiP1 minimal promoter element of the bidirectional ZmUbi1 promoter expressed YEP at similar expression levels as compared to Z. mays callus transformed with a binary plasmid containing a unidirectional ZmUbi1 promoter driving the yfp coding sequence (pDAB101556; see FIG. 12).

### Example 8

### Construct of a Four-Gene Cassette Stack

A plasmid pDAB105803 construct, is used as the starting plasmid to generate a four-gene cassette cassette stack (aad1-2a-Phiyfp and cry34-2a-cry35) driven by single Zea mays Ubiquitin-1 bi-directional promoter. A representative map of plasmid pDAB105803 is shown in FIG. 16, which contains a Zea mays Ubiquitin-1 bi-directional promoter.

The aad1-2a-Phiyfp fragment derived from plasmid pDAB105841 is cloned into the BamHI and SacI cut vector backbone of the plasmid pDAB105803 using cloning methods commonly known in the art. This resulted in the intermediate plasmid pDAB105842 (FIG. 17). A NotI/XbaI digested cry34(8V6)-2a-cry35 fragment obtained from the plasmid pDAB105840 is cloned between NotI/SpeI sites of plasmid pDAB105842 to construct plasmid pDAB105843. The plasmid pDAB105843 contains cry34(8V6)-2a-cry35 and aad1-2a-Phiyfp gene cassettes on each side of ZmUbi1 bidirectional promoter (FIG. 18).

A binary vector containing the ZmUbi1 bidirectional promoter, and gene expression cassettes cry34(8\76)-2a-cry35 and Phiyfp-2a-aad1 from plasmid pDAB105842 is generated via a GATEWAY L-R CLONASE reaction (Invitrogen, Carlsbad, Calif.) with a destination plasmid pDAB101917, The resulting vector, pDAB108717, contains the cry34(8V6)-2a-cry35aad1-2a-Phiyfp, and PAT gene expression cassettes within the T-DNA borders (FIG. 19).

### Example 9

### Construct of a Second Four-Gene Cassette Stack

A plasmid pDAB105803 construct is used to generate a second four-gene cassette stack (Phiyfp-2a-aad1 and cry34-2a-cry35) driven by single Zea mays Ubiquitin-1 bi-directional promoter. A Phiyfp-2a-aad1 fragment derived from plasmid pDAB105844 is cloned into the BamHI and SacI cut vector backbone of the plasmid pDAB105803 using cloning methods commonly known in the art. This resulted in the intermediate plasmid pDAB105845 (FIG. 20). A NotI/XbaI digested cry34(8V6)-2a-cry35 fragment obtained from the plasmid pDAB105840 is cloned between NotI/SpeI sites of plasmid pDAB105845 to construct plasmid pDAB105846 (FIG. 21). The plasmid pDAB105846 contains cry34(8V6)-2a-cry35 and Phiyfp-2a-aad1 gene cassettes on each side of the ZmUbi1 bidirectional promoter.

A binary vector containing the ZmUbi1 bidirectional promoter, and gene cassettes cry34(8V6)-2a-cry35 and Phiyfp-2a-aad1 from plasmid pDAB105846 is generated via a GATEWAY L-R, CLONASE reaction (Invitrogen, Carlsbad, Calif.) with a destination plasmid pDAB101917. The resulting vector, pDAB108718, contains the cry34(8V6)-2a-cry35, Phiyfp-2a-aad1, and PAT gene expression cassettes within the T-DNA borders (FIG. 21).

### Example 10

### Transformation of Agrobacterium tumefaciens Strain :DAt13192

The pDAB108717 and pDAB108718 binary vectors are transformed into Agrobacterium tumefaciens ternary strain DAt13192 (see U.S. Prov. Pat. App. No. 61/368965, the content of which is hereby incorporated by reference in its entirety). Bacterial colonies are isolated and binary plasmid DNA is extracted and verified via restriction enzyme digestions.

### Example 11

### Transformation into Maize

Ear Sterilization and Embryo Isolation: To obtain maize immature embryos, plants of Zea mays (c.v. B104) are grown in the greenhouse and self or sib-pollinated to produce ears. The ears are harvested approximately 9-12 days post-pollination. On the day of the experiment, ears are surface-sterilized by immersion in a 20% solution of household bleach, which contains 5% sodium hypochlorite, and shaken for 20-30 minutes, followed by three rinses in sterile water. After sterilization, immature zygotic embryos (1.5-2.2 mm) are aseptically dissected from each ear and randomly distributed into micro-centrifuge tubes containing liquid infection media (LS Basal Medium, 4.43 g/L; N6 Vitamin Solution [1000×], 1.00 mL/L; L-proline, 700.0 mg/L; sucrose, 68.5 g/L; glucose, 36.0 g/L; 2,4-D, 1.50 mg/L. For a given set of experiments, pooled embryos from 2-3 ears are used for each treatment.

Agrobacterium Culture Initiation: Glycerol stocks of Agrobacterium strains containing the binary vectors described above are streaked on AB minimal medium plates containing appropriate antibiotics and are grown at 20° C. for 3-4 days. A single colony is picked and streaked onto YEP plates containing the same antibiotics and is incubated at 28° C. for 1-2 days.

Agrobacterium Culture and Co-cultivation: On the day of the experiment, Agrobacterium colonies are picked from the YEP plate, suspended in 10 mL of infection medium in a 50 mL disposable tube, and the cell density is adjusted to OD₆₀₀=0.2-0.4 nm using a spectrophotometer. The Agrobacterium cultures are placed on a rotary shaker at 115 rpm, room temperature. While embryo dissection is performed. Immature zygotic embryos between 1.5-2.2 mm in size are isolated from the sterilized maize kernels and placed in 1 mL of the infection medium and washed once in the same medium. The Agrobacterium suspension (2 mL) is added to each tube and the tubes were inverted for about 20 times then shaken for 10-15 minutes. The embryos are transferred onto co-cultivation media (MS Salts, 4.33 g/L; L-proline, 700.0 mg/L; myo-inositol, 100.0 mg/L; casein enzymatic hydrolysate 100.0 mg/L; Dicamba 3.30 mg/L; sucrose, 30.0 g/L; Gelzan™, 3.00 g/L; modified MS-Vitamin [1000×], 1.00 ml/L; AgNo₃, 15.0 mg/L; Acetosyringone, 100.0 μM), oriented with the scutenum facing up, and incubated for 3-4 days in the light at 25° C.

YFP/Phiyfp Transient expression: Transient YFP/Phiyfp expression can be observed in transformed embryos after 3 days of co-cultivation with Agrobacterium. The embryos are observed under a stereomicroscope (Leica Microsystems, Buffalo Grove, Ill.) using YFP filter and 500 nm light source.

Callus Selection and Regeneration of Putative Events: Following the co-cultivation period, embryos are transferred to resting media (MS salts, 4.33 g/L; L-proline, 700.0 mg/L; myoinositol, 100.0 mg/L, MES [(2-(n-morpholino)-ethanesulfonic acid), free acid], 500.0 mg/L; casein enzymatic hydrolysate, 100.0 mg/L; Dicamba, 3.30 mg/L; sucrose, 30.0 g/L; Gelzan™, 2.30 g/L; modified MS-Vitamin [1000×], 1.00 ml/L; AgNO₃, 15,0 mg/L; Carbenicillin, 250.0 mg/L) without selective agent and incubated in 24 hours light with light intensity of 50 μmol m⁻²s⁻¹ for 7 days at 28° C. Embryos are transferred onto selection 1 media (MS salts, 4.33 g/L; L-proline, 700.0 mg/L; myo-inositol, 100.0 mg/L; MES [(2-(n-morpholino)-ethanesulfonic acid), free acid], 500.0 mg/L; casein enzymatic hydrolysate, 100.0 mg/L; Dicamba, 3.30 mg/L; sucrose, 30.0 g/L; Gelzan™, 2.30 g/L; modified MS-Vitamin [1000×], 1.00 ml/L; AgNO₃, 15.0 mg/L; Carbenicillin, 250.0 mg/L), containing 3 mg/L Bialaphos and incubated in 24 hours light with light intensity of 50 μmol m⁻²s⁻¹ for 7 days at 28° C.

Embryos with proliferating embryogenic calli are transferred onto selection 2 media (MS salts, 4.33 g/L; myo-inositol, 100.0 mg/L; L-proline, 700.0 mg/L; MES [(2-(n-morpholino)-ethanesulfonic acid), free acid], 500.0 mg/L; casein enzymatic hydrolysate, 100.0 mg/L; Dicamba, 3.30 mg/L; sucrose, 30.0 g/L; Gelzan™ 2.30 g/L; modified MS-Vitamin [1000×], 1.00 ml/L; AgNo₃, 15.0 mg/L; Carbenicillin, 250.0 mg/L), containing 5 mg/L Bialaphos and are incubated in 24 hours light with light intensity of 50 μmol m⁻²s⁻¹ for another 14 days at 28° C. This selection step allows transgenic callus to further proliferate and differentiate. The callus selection period may last for three weeks. Proliferating, embryogenic calli are transferred onto regeneration 1 media (MS salts, 4.33 g/L; myo-inositol, 100.0 mg/L; L-proline, 350.0 mg/L; MES [(2-(n-morpholino)-ethanesulfonic acid), free acid]; 250.0 mg/L; casein enzymatic hydrolysate, 50.0 mg/L; NAA, 0,500 mg/L; ABA, 2.50 mg/L; BA, 1.00 mg/L; sucrose, 45.0 g/L; Geizan™ 2.50 g/L; modified MS-Vitamin [1000×], 1.00 mi/L; AgNO₃, 1.00 mg/L; Carbenicillin, 250.0 mg/L), containing 3 mg/L Bialaphos and cultured in 24 hours light with light intensity of 50 μmol m⁻²s⁻¹ for 7 days at 28° C.

Embryogenic calli with shoot/buds are transferred onto regeneration 2 media (MS salts, 4.33 g/L; modified MS-Vitamin [1000×], 1.00 ml/L; myo-inositol, 100.0 mg/L; sucrose, 60.0 g/L; Gellan Gum G434™, 3.00 g/L; Carbenicillin, 250.0 mg/L), containing 3 mg/L Bialaphos. The cultures are incubated under 24 hours light with light intensity of 50 μmol⁻²s⁻¹ for 7-10 days at 28° C. Small shoots with primary roots are transferred to shoot elongation and rooting media (MS salts, 4.33 g/L; N6 Vitamin Solution [1000×], 1.00 mL/L; myo-inositol, 100.0 mg/L; sucrose, 30.0 g/L; agar 5.50 g/L; in phytatray and are incubated under 16/8 hours light/dark at 90 μmol m⁻²s⁻¹ for 7 days at 28° C. Healthy putative transgenic plantlets are selected then incubated in 16/8 hours light/dark at 200 μmol m⁻²s⁻¹ for another 2-5 days at 25° C. and are analyzed for transgene copy number and transferred to the greenhouse.

### Example 12

### Transient Phiyfp Expression

Transient expression of Phiyfp from Zea mays embryos transformed with pDAB108717 is performed. The bi-directional ZmUbi1 promoter can express Phiyfp from aad1-2a-Phiyfp gene expression cassette, where non-transformed embryo does not show any Phiyfp fluorescence. Similar level of Phiyfp expression can be observed from Zea mays embryos transformed with a binary plasmid pDAB105748 (FIG. 15) containing a uni-directional Zea mays (Zm) Ubi1 promoter driving single Phiyfp coding sequence displayed expected level of YFP/Phiyfp expression. Transient expression of Phiyfp can be observed from Zea Mays embryos transformed with pDAB108718, where bi-directional ZmUbi1 promoter can express Phiyfp from the Phiyfp-2a-aad1 gene expression cassette.

### Example 13

### Phiyfp Expression in Stably Transformed Maize

Phiyfp Expression in Stably Transformed Zea mays Callus Driven by a Bi-Directional Zm Ubi1 Promoter: Zea Mays embryos transformed with the pDAB108717 binary vector containing the aad1-2a-Phiyfp gene expression cassette show good Phiyfp expression. The bi-directional Zm Ubi1 promoter can drive robust expression of Phiyfp. These results confirm that the Min-UbiP1 minimal promoter element of the bi-directional Zm Ubi1 promoter is capable of expressing a reporter gene, for example Phiyfp or YFP. The levels of expression of the Phiyfp protein are similar as compared to Zea mays callus transformed with a control binary vector which contained the uni-directional Zm Ubi1 promoter driving the Phiyfp coding sequence (pDAB105748). Expression of Phiyfp is not detected in the negative control callus which is not transformed with a binary construct and did not contain the PhiyfP coding sequences.

Zea mays embryos transformed with the pDAB108718 binary vector which contains the Phiyfp-2a-aad1 gene expression cassette show good Phiyfp expression. The bi-directional Zm Ubi1 promoter can drive robust expression of Phiyfp. These results confirm that the Min-UbiP1 minimal promoter element of the bi-directional Zm Ubi1 promoter is capable of expressing a reporter gene, for example Phiyfp or YFP.

### Example 14

### Estimation of Transgene Copy Number

Transgene Copy Number Estimation Using Real Time TaqMan™ PCR: Zea mays plants were transformed with binary vectors containing a bidirectional Zm Ubi1 promoter, pDAB1.08717and piDAB108718, and other plants are transformed with a control binary vector, pDAB105748. The presence of coding sequence (Phiyfp, aad1, cry34, cry35, Pat) within the genome of Z. mays plants transgenic to pDAB108717 and pDAB1.08718 was confirmed via a TaqMan hydrolysis probe assay. The plants transgenic to control vector pDAB105748 were analyzed for the presence of Phiyfp sequence. Stably-transformed transgenic Z. mays plantlets that developed from the callus were obtained and analyzed to identify events that contained a low copy number (1-2 copies) of full-length T-strand inserts from the pDAB108717 and pDAB108718 binary vectors, and pDAB105748 control binary vector. Confirmed plantlets were advanced to the green house and grown.

The Roche Light Cycler480™ system was used to determine the transgene copy number for events that were transformed with the pDAB108717 and pDAB108718 binary vector. The method utilized a biplex Taq:Man™ reaction that employed oligonucleotides specific to the coding sequence and to the endogenous Z. mays reference gene, invertase (Genbank Accession No: U16123.1), in a single assay. Copy number and zygosity were determined by measuring the intensity of coding sequence-specific fluorescence, relative to the invertase-specific fluorescence, as compared to known copy number standards.

TABLE 7
Forward and reverse nucleotide primer and fluorescent probes
synthesized by Integrated DNA Technologies, Coralville, IA).

| Primer Name | Primer Sequence |
|---|---|
| YFP Forward Primer | GATGCCTCAGTGGGAAAGG (SEQ ID NO: 7) |
| YFP Reverse Primer | CCATAGGTGAGAGTGGTGACAA (SEQ ID NO: 8) |
| YFP Probe | ROCHE UPL Probe #125 CTTGGAGC (SEQ ID NO: 40) |
| | Cat# 04693604001 (Roche, Indianapolis, IN) |
| Invertase Forward Primer | TGGCGGACGACGACTTGT (SEQ ID NO: 9) |
| Invertase Reverse Primer | AAACTTTTGGAGGCTGCCGT (SEQ ID NO: 10) |
| Invertase Probe | 5′HEX/CGAGCAGACCGCCGTGTACTTCTACC/3BHQ_1/3′ |
| | (SEQ ID NO: 11) |
| AAD1 Forward Primer | TGTTCGGTTCCCTCTACCAA (SEQ ID NO: 12) |
| AAD1 Reverse Primer | CAACATCCATCACCTTGACTGA (SEQ ID NO: 13) |
| AAD1 Probe | CACAGAACCGTCGCTTCACTCAACA (SEQ ID NO: 14) |
| Cry34 Forward Primer | GCCAACGACCAGATCAAGAC (SEQ ID NO: 41) |
| Cry34 Reverse Primer | GCCGTTGATGGAGTAGTAGATGG (SEQ ID NO: 42) |
| Cry34 Probe | CCGAATCCAACGGCTTCA (SEQ ID NO: 43) |
| Cry35 Forward Primer | CCTCATCCGCCTCACCG (SEQ ID NO: 44) |
| Cry35 Reverse Primer | GGTAGTCCTTGAGCTTGGTGTC (SEQ ID NO: 45) |
| Cry35 Probe | CAGCAATGGAACCTGACGT (SEQ ID NO: 46) |
| PAT Forward Primer | ACAAGAGTGGATTGATGATCTAGAGAGGT (SEQ ID NO: 47) |
| PAT Reverse Primer | CTTTGATGCCTATGTGACACGTAAACAGT (SEQ ID NO: 48) |
| PAT Probe | GGTGTTGTGGCTGGTATTGCTTACGCTGG (SEQ ID NO: 49) |

For Z. mays samples transformed with the pDAB108717 and pDAB108718 binary vectors, a coding sequence-specific DNA fragment is amplified with one TaqMan® primer/probe set containing a probe labeled with FAM fluorescent dye, and invertase is amplified with a second TaqMan™ primer/probe set containing a probe labeled with HEX fluorescence (Table 7) The PCR reaction mixture is prepared as set forth in Table 8, and the gene-specific DNA fragments are amplified according to the conditions set forth in Table 9. Copy number and zygosity of the samples are determined by measuring the relative intensity of fluorescence specific for the coding sequence to fluorescence specific for the reference gene, invertase, as compared to known copy number standards.

Standards are created by diluting the vector (pDAB108717 and pDAB108717) into Z. mays B104 genomic DNA (gDNA) to obtain standards with a known relationship of vector:gDNA. For example, samples having one, two, and four cop(ies) of vector DNA per one copy of the Z. mays B104 gDNA are prepared. One and two copy dilutions of the vector mixed with the Z. mays B104 gDNA standard are validated against a control Z. mays event that is known to be hemizygous, and a control Z. mays event that is known to be homozygous (Z. mays event 278; See PCT International Patent Publication No. WO 2011/022469 A2, the content of which is hereby incorporated by reference in its entirety). A TaqMan® biplex assay which utilizes oligonucleotides specific to the coding sequence gene and oligonucleotides specific to the endogenous Z. mays reference gene, invertase, is performed by amplifying and detecting a gene-specific DNA fragment for coding sequence with one TaqMan® primer/probe set containing a probe labeled with RAM fluorescent dye, and by amplifying and detecting a gene-specific DNA fragment for invertase with a second TaqMan® primer/probe set containing a probe labeled with HEX fluorescence. According to Table 7, the coding sequence TaqMan® reaction mixture is prepared as set forth in Table 8 and the specific fragments are amplified according to the conditions set forth in Table 9.

TABLE 8
Taqman ® PCR reaction mixture.

| | | Final |
|---|---|---|
| Number of Reactions | μl each | Concentration |
| H₂O | 0.5 μL | — |
| PVP (10%) | 0.1 μL | 0.1% |
| ROCHE 2X Master Mix | 5.0 μL | 1X |
| Coding sequence Forward Primer (10 μM) | 0.4 μL | 0.4 μM |
| Coding sequence Reverse Primer (10 μM) | 0.4 μL | 0.4 μM |
| Coding sequence Probe UPL#125 (5 μM) | 0.4 μL | 0.2 μM |
| Invertase Forward Primer (10 μM) | 0.4 μL | 0.4 μM |
| Invertase Reverse Primer (10 μM) | 0.4 μL | 0.4 μM |
| Invertase Probe (5 μM) | 0.4 μL | 0.2 μM |
| Template DNA | 2.0 μL | — |
| Total reaction volume | 10 μL | — |

The level of fluorescence generated for each reaction is analyzed using the Roche LightCycler 480™ Thermocycler according to the manufacturer's directions. The FAM fluorescent moiety is excited at an optical density of 465/510 nm, and the HEX fluorescent moiety is excited at an optical density of 533/580 nm. The copy number can be determined by comparison of Target/Reference values for unknown samples (output by the LightCycler 480™) to Target/Reference values of four known copy number standards (for example, Null, 1-Copy (hemi), 2-Copy (homo), and 4-Copy).

TABLE 9
Thermocycler conditions for PCR amplification.

| PCR Steps | Temp (° C.) | Time | No. of cycles |
|---|---|---|---|
| Step-1 | 95 | 10 minutes | 1 |
| Step-2 | 95 | 10 seconds | 40 |
| | 59 | 35 seconds | |
| | 72 | 1 second | |
| Step-3 | 40 | 11 seconds | 1 |

Results from the transgene copy number analysis of transgenic plants obtained via transformation with a bidirectional ZmUbi1 promoter constructs (pDAB108717 and pDAB108718), and of transgenic plants obtained via transformation with a control unidirectional ZmUbi1 promoter Phiyfp construct (pDAB105748) are summarized in Table 10. Only plants with 1-2 copies of the all transgenes are transferred to the greenhouse for further expression analyses.

TABLE 10
Transgene copy number estimation of the transgenic plants obtained from
bidirectional promoter and control constructs.

| | Number of Embryos | Number of | 1-2 Copies |
|---|---|---|---|
| Construct | Transformed | Positive Events | of all genes |
| pDAB108717 | 314 | 66 | 14 |
| pDAB108718 | 252 | 63 | 10 |
| pDAB105748 | 32 | 8 | 2 |

### Example 15

### Stable Phiyfp Expression in Maize T0 Plants

Stable Phiyfp Expression in Zea mays T₀ Plants Driven by bidirectional Zm Ubi1 Promoter: Zea mays embryos transformed with the pDAB108717 binary vector containing the aad1-2a-Phiyfp gene expression cassette can be observed. The bi-directional Zm Ubi1 promoter can drive robust expression of the Phiyfp both in shoot and root tissues. The results confirm that the Min-UbiP1 minimal promoter element of the bi-directional Zm Ubi1 promoter is capable of expressing a reporter gene, for example Phiyfp or YFP that is bicistronically fused with aad1 using a 2A sequence. The levels of expression of the Phiyfp protein is similar to Z. mays embryos transformed with a control binary vector which contains the uni-directional Zm Ubi1 promoter driving the Phiyfp coding sequence (pDAB105748). Expression of Phiyfp is not detected in the negative control plants which are not transformed with a binary construct and do not contain the Phiyfp coding sequences.

Phiyfp expression in leaf and root tissues of Zea mays T0 plants transgenic to pDAB108718 binary vector which contains the Phiyfp-2a-aad1 gene expression cassette can be observed. The bi-directional Zm Ubi1 promoter can drive robust expression of Phiyfp. The results confirm that the Min-UbiP1 minimal promoter element of the bi-directional Zm Ubi1 promoter is capable of expressing a reporter gene, for example Phiyfp or YFP fused to aad-1 with a 2A sequence.

### Example 16

### Cry34, Cry35, and AAD1 Protein Analysis

Plants are sampled into columns 1-10 of a matrix box in 1.5 mL conical tubes to which 1 steel bead is added followed by PBST+0.5% BSA (0.6 mL). The box is then bead heated for sample grinding in a Geno Grinder for 5 minutes at 1500 rpm then centrifuged at 3700 rpm for 7 minutes at 4° C.

Cry34/35 ELISA assay: In a separate, 96 deep well plate, a sample of the extract is diluted 1:200 in PBST+1% blotto. Two volumes of 25 of the diluted sample are then transferred to separate 96-well plates that have been arrayed with anti-Cry34 and anti-Cry35 (Meso Scale Discovery). In the 11 and 12 columns of each plate standard concentrations of Cry34 and Cry35 in PBST+1% blotto are added (25 μL). The plates are then incubated while shaking at room temperature for one hour. The plates are then washed with PBST (3×300 μL). Then 25 μL of a solution of SulfoTAG conjugated anti-Cry34 and anti-Cry35 is added to each well and incubated with shaking at room temperature for one hour. The plates are then washed with PBST (3×300 μL). A volume of 150 μL Read Buffer T (Meso Scale Discovery) is then added and the plate is immediate read on a SECTOR® 6000 reader. Concentrations of proteins in the sample can be calculated using the standard curve for the respective protein generated from the same plate.

AAD-1 ELISA assay: In a separate, 96 deep well plate, a sample of the extract is diluted 1:20 in PBST+0.5% BSA. Two volumes of 200 μL of the diluted sample are then transferred to separate 96 well plates that have been coated with anti-AAD1 (provided by Acadia Bioscience LLC). In the 11 and 12 columns of each plate standard concentrations of AAD1 in PBST+0.5% BSA are added (200 μL). A volume of 50 μL of biotinylated anti-AAD1 is then added to each well and the plates are incubated while shaking at room temperature for one hour. The plates are then washed with PBST (5×300 μL). Then 100 μL of a steptavidin-alkaline phosphate conjugate solution is added to each well and incubated with shaking at room temperature for 30 minutes. The plates are then washed with PBST (5×300 μL ). A volume of 100 μL substrate (p-nitrophenylphosphate, PNPP) is then added and incubated with shaking at room temperature for 45 minutes. The plates are then read at A405 on a SpectraMax M5 plate reader (Molecular Devices). Concentrations of proteins in the sample can be calculated using the standard curve generated from the same plate.

### Example 17

### Protein Analysis of Maize T0 Plants

Protein analysis of maize T0 plants driven by the bi-directional Zea mays Ubiquitini Promoter construct (pDAB108717): Representative ELISA analysis of 11 transgenic T0 maize plants obtained from Zea mays embryos transformed with pDAB108717 that contains cry34-2a-cry35 and aad1-2a-Phiyfp is summarized in Table 11. Bi-directional Zm Ubi1 promoter show robust expression of both Cry34 and Cry35 coding sequences in leaf. Surprisingly, the protein data demonstrate up to 4-fold higher expression of Cry34 from bidirectional construct pDAB108717 compared to unidirectional Zm Ubi1-driven construct. A similar 8-10 fold higher expression of Cry35 and AAD1 proteins is also unexpectedly observed from bidirectional construct pDAB108717 compared to unidirectional Zm Ubi1-driven construct. These observations show that the single ZmUbiquitini bidirectional promoter in construct pDAB10871,7 can express multiple genes Cry34, Cry35, and AAD1) at unexpectedly higher levels as compared to Zea mays plants transformed with a binary plasmid which contains uni-directional Zm Ubi1 promoter driving the same genes, where each coding sequence is driven by an independent Zm Ubi1 promoter.

Cry34 and Cry35 expression correlation of maize T0 plants driven by the bi-directional Zea mays Ubiquitin1 Promoter construct (pDA13108717): The correlation analysis between Cry34 and Cry35 proteins in 11 transgenic T0 maize plants obtained from Zea mays embryos transformed with pDAB108717 that contains cry34-2a-cry35 is shown in FIG. 23A. A very high correlation (R Square=0.98) demonstrates strong expression co-regulation between Cry34 and Cry 35 from the cry34-2a-cry35 gene expression cassette driven by the bi-directional Zm Ubi1 promoter.

TABLE 11
Cry34/Cry35/AAD1 expression in T0 maize
pDAB108717 transgenic plants

| Plant ID | Cry34 ng/cm² | Cry35 ng/cm² | AAD1 ng/cm² |
|---|---|---|---|
| 108717[1]-032.001 | 277 | 294 | 137 |
| 108717[3]-067.001 | 85 | 93 | 130 |
| 108717[2]-137.001 | 427 | 467 | 6 |
| 108717[1]-027.001 | 484 | 563 | 185 |
| 108717[1]-036.001 | 0 | 0 | −7 |
| 108717[2]-107.001 | 219 | 296 | 112 |
| 108717[2]-113.001 | 0 | 0 | −12 |
| 108717[2]-115.001 | 160 | 175 | 68 |
| 108717[2]-118.001 | 196 | 179 | −5 |
| 108717[2]-125.001 | 318 | 335 | 193 |
| 108717[2]-127.001 | 115 | 127 | 101 |
| Zm Ubi-Cry34/Cry35 | 110 | 67 | 18 |

Protein analysis of maize T0 plants driven by the bi-directional Zea mays Ubiquitini Promoter construct (pDAB108718): Representative ELISA analysis of 11 transgenic T0 maize plants obtained from Zea mays embryos transformed with pDAB108718 that contains cry34-2a-cry35 is summarized in Table 12, Bi-directional ZmUbi1 promoter showed robust expression of both Cry34 and Cry35 coding sequences in leaf. The protein data demonstrate several fold higher expression of Cry34, Cry35 and AAD1 proteins from bidirectional construct pDAB108718 as compared to unidirectional Zm Ubi1-driven construct. These observations confirm that the Zea mays Ubiquitini bidirectional promoter in construct pDAB108718 expressed multiple genes (e.g., Cry34, Cry35, and AAD1) at unexpectedly higher levels as compared to Zea mays plants transformed with a binary plasmid which contains uni-directional Zm Ubi1 promoter driving the same genes, where each coding sequence driven by an independent Zm Ubi1 promoter.

Cry34 and Cry35 expression correlation of maize T0 plants driven by the bi-directional Zea mays Ubiquitini Promoter construct (pDAB108718): The correlation analysis between Cry34 and Cry35 proteins in 11 transgenic T0 maize plants obtained from Zea mays embryos transformed with pDAB108718 that contains cry34-2a-cry35 is shown in FIG. 23B. A very high correlation (R Square=0.98) demonstrates strong expression co-regulation between Cry34 and Cry 35 from the cry34-2a-cry35 gene expression cassette driven by the bi-directional Zm Ubi1 promoter.

TABLE 12
Cry34/Cry35/AAD1 expression in T0 maize pDAB108718
transgenic plants

| Plant ID | Cry34 ng/cm² | Cry35 ng/cm² | AAD1 ng/cm² |
|---|---|---|---|
| 108718[3]-060.001 | 0 | 0 | −9 |
| 108718[3]-048.001 | 129 | 155 | 72 |
| 108718[2]-106.001 | 0 | 0 | −8 |
| 108718[3]-061.001 | 78 | 109 | 0 |
| 108718[3]-049.001 | 28 | 11 | −5 |
| 108718[3]-053.001 | 128 | 175 | 2 |
| 108718[1]-024.001 | 157 | 186 | 0 |
| 108718[2]-083.001 | 177 | 205 | 42 |
| 108718[2]-085.001 | 642 | 642 | 32 |
| 108718[2]-089.001 | 127 | 139 | 50 |
| 108718[2]-091.001 | 175 | 168 | 58 |
| 108718[2]-100.001 | 181 | 188 | 104 |
| Zm Ubi-Cry34/Cry35 | 110 | 67 | 18 |

### Example 18

### Transgene Stacking: Synthetic Bidirectional Promoters (T1 data)

Gene expression of T1 plants driven by the bi-directional Zea mays Ubiquitin1 Promoter constructs: ten to twelve single copy events per construct are selected for analysis except that the control construct pDAB108716 has only one event. Five plants/events for the V6 stage are tested and three plants/events for the V10-12 and/R3 stages are tested. Protein assays are performed using LCMS or ELISA.

The constructs used in this example are shown in FIG. 26. pDAB108706 (ZMUbi bidirectional (−200)) and pDAB108707 (ZMUbi bidirectional (−90)) are constructs with representative bidirectional promoter of the present invention; pDAB101556 (ZmUbi1-YFP control) and pDAB108716 (ZMUbi1 without minimal promoter) serve as control constructs with uni-directional promoters.

Exemplary expression results (V6) from the four constructs for YFP protein (LCMS) in ng/cm2 are shown in FIG. 27A, and exemplary relative expression results (V6) from the four constructs for YFP RNA are shown in FIG. 27B.

Exemplary expression results (V6) from the four constructs for GUS protein (LCMS) in ng/cm2 are shown in FIG. 28A, and exemplary relative expression results (V6) from the four constructs for GUS RNA are shown in FIG. 28B.

Exemplary expression results (V6) from the four constructs for AAD1 protein (LCMS) in ng/cm2 are shown in FIG. 29A, and exemplary relative expression results (V6) from the four constructs for AAD1 RNA are shown in FIG. 29B.

A statistical analysis of expression results (V6) from the four constructs for YFP protein (LCMS) in ng/cm2 is shown in FIG. 30A, and the mean values for pDAB108707, pDAB108706, pDAB101556, and pDAB108716 are 57.63, 52.66, 49.75, and 0 respectively. A statistical analysis of relative expression results (V6) from the four constructs for YFP RNA is shown in FIG. 30B, and the mean values for pDAB108706, pDAB108707, pDAB101556, and pDAB108716 are 9.96, 8.07, 6.95, and 1.01 respectively.

A statistical analysis of expression results (V6) from the four constructs for GUS protein (LCMS) in ng/cm2 is shown in FIG. 31A, and the mean values for pakB108706, pDAB108707, pDAB101556, and pDAB108716 are 151.27, 143.22, 0, and 213.17 respectively. A statistical analysis of relative expression results (V6) from the four constructs for GUS RNA is shown in FIG. 31B, and the mean values for pDAB108706, pDAB108707, pDAB101556, and pDAB108716 are 0.65, 0.78, 0, and 3.03 respectively.

A statistical analysis of expression results (V6) from the four constructs for AAD1 protein (LCMS) in ng)cm2 is shown in FIG. 32A, and the mean values for pDAB108706, pDAB108707, pDAB101556, and pDAB108716 are 710.88, 1417,01, 856,58, and 1795.43 respectively. A statistical analysis of relative expression results (V6) from the four constructs for AAD1 RNA is shown in FIG. 32B, and the mean values for pDAB108706, pDAB108707, pDAB101556, and pDAB108716 are 1.33, 1.37, 1.93, and 2.93 respectively.

FIGS. 33A, 33B, and 33C show exemplary expression results (V10) from the four constructs for YFP, AAD 1, and GUS protein (LCMS) in ng/cm2 respectively,

FIGS. 34A, 34B, and 34C show statistical analysis of expression results (V10) from the four constructs for YFP, GUS, and AAD1 protein (LCMS) in ng/cm2 respectively. The mean values for pDAB108706, pDAB108707, pDAB101556, and pDAB108716 for YFP (FIG. 34A) are 71.77, 81.81, 49.58, and 23.01 respectively. The mean values for pDAB108706, pDAB108707, pDAB101556, and pDAB108716 for GUS (FIG. 34B) are 109.63, 98.25, 0, and 138.02 respectively. The mean values for pDAB108706, pDAB108707, pDAB101556, and pDAB108716 for AAD1 (FIG. 34C) are 666.11, 597.80, 715,12, and 1002,84 respectively.

FIGS. 35A, 35B, and 35C show exemplary expression results (R3) from the four constructs for YFP, GUS, and AAD1 protein (LCMS) in ng/cm2 respectively.

FIGS. 36A, 36B, and 36C show statistical analysis of expression results (R3) from the four constructs for YFP, GUS, and AAD1 protein (LCMS) in ng/cm2 respectively. The mean values for pDAB108706, pDAB108707, pDAB101556, and pDAB108716 for YFP (FIG. 36A) are 91.38, 49.49, 21.67, and 0.40 respectively. The mean values for pDAB108706, pDAB108707, pDAB101556, and pDAB108716 for GUS (FIG. 36B) are 5.52, 16.81, 1.07, and 46.60 respectively. The mean values for pDAB108706, pDAB108707, pDAB101556, and pDAB108716 for AAD1 (FIG. 36C) are 156.71, 153.44, 165.40, and 197.80 respectively.

The results show that maize Ubi1 bidirectional promoters of the present invention can drive robust expression of GUS and YFP, where the YFP expression from Maize Ubi1 bidirectional promoter is similar to unidirectional maize Ubi1 driven YFP. The results also suggest that bidirectional transcription has non-significant effect on GUS expression (GUS expression compared to the constructs lacking minimal promoter without YFP expression

### Example 19

### A Combination of Bidirectional Promoter and 2A Bicistronic Sequence to Drive Four Transgenes from One Single Promoter (T1 Data)

Gene expression of T1 plants driven by the bi-directional Zea mays Ubiquitini Promoter constructs: ten to twelve single copy events per construct are selected for analysis except that the control constructs have four or five events per construct. Five plants/events for the V6 stage are tested and three plants/events for the V10-12 and/R3 stages are tested. Protein assays are performed using LCMS or ELISA.

FIG. 37A shows exemplary relative expression results (V6) of Cry34 RNA from the four constructs pDAB105748 (ZMUbi1-YFP), pDAB105818 (ZMUbi1-Cry341ZMUbi1-Cry35/ZMUbi1-AAD1), pDAB108717 (YFP/AAD-1-ZMUbi1 bidirectional-Cry34-Cry:35), and pDAB108718 (AAD1/YFP-ZMUbi1 bidirectinal-Cry34-Cry35). FIG. 37B shows exemplary relative expression results (V6) of Cry34 protein (LCMS) from the same four constructs pDAB105748, pDAB105818, pDAB10871.7, and pDAB108718.

FIG. 38A shows exemplary relative expression results (V6) of AAD1 RNA from the four constructs pDAB105748, pDAB105818, pDAB108717, and pDAB108718. FIG. 38B shows exemplary relative expression results (V6) of AAD1 protein (LCMS) from the same four constructs pDAB105748, pDAB105818, pDAB108717, and pDAB108718.

FIG. 39A shows exemplary relative expression results (V6) of YFP RNA from the four constructs pDAB105748, pDAB105818, pDAB108717, and pDAB108718. FIG. 39B shows exemplary relative expression results (V6) of YFP protein (LCMS) from the same four constructs pDAB105748, pDAB105818, pDAB108717, and pDAB108718.

FIG. 40A shows exemplary relative expression results (V6) of Cry35 RNA from the four constructs pDAB105748, pDAB105818, pDAB108717, and pDAB108718. FIG. 40B shows exemplary relative expression results (V6) of Cry35 protein (ELISA) from the same four constructs pDAB105748, pDAB105818, pDAB108717, and pDAB108718.

FIG. 41 shows exemplary relative expression results (V6) of PAT RNA from the four constructs pDAB105748, pDAB105818, pDAB108717, and pDAB108718.

FIG. 42A shows a statistical analysis of expression results (V6) of Cry34 RNA from the four constructs pDAB105748, pDAB105818, pDAB108717, and pDAB108718 with the mean values 0, 2.42, 2.67, and 2.25 respectively. FIG. 42B shows a statistical analysis of expression results (V6) of Cry34 protein from the same four constructs pDAB105748, pDAB105818, pDAB108717, and pDAB108718 with the mean values 0, 596.94, 2044.73, and 719.18 respectively.

FIG. 43A shows a statistical analysis of expression results (V6) of AAD1 RNA from the four constructs pDAB105748, pDAB105818, pDAB108717, and pDAB108718 with the mean values 0, 1.98, 2,68, and 2,03 respectively. FIG. 43B shows a statistical analysis of expression results (V6) of AAD1 protein from the same four constructs pDAB105748, pDAB105818, pDAB108717, and pDAB108718 with the mean values 0.2237.54, 5763.88, and 2379.15 respectively.

FIG. 44A shows a statistical analysis of expression results (V6) of YFP RNA from the four constructs pDAB105748, pDAB105818, pDAB108717, and pDAB108718 with the mean values 3.59, 0, 2.78, and 1.95 respectively. FIG. 44B shows a statistical analysis of expression results (V6) of YFP protein from the same four constructs pDAB105748, pDAB105818, pDAB108717, and pDAB108718 with the mean values 1420.69, 251.68, 1154.04, and 706.04 respectively.

FIG. 45A shows a statistical analysis of expression results (V6) of Cry35 RNA from the four constructs pDAB105748, pDAB105818, pDAB10871.7, and pDAB108718 with the mean values 0, 1.12, 3.74, and 3.20 respectively. FIG. 45B shows a statistical analysis of expression results (V6) of Cry35 protein from the same four constructs pDAB105748, pDAB105818, pDAB108717, and pDAB108718 with the mean values 0, 283.54, 635.83, and 90.97 respectively.

FIG. 46 shows a statistical analysis of expression results (V6) of PAT RNA from the four constructs pDAB105748, pDAB105818, pDAB10871.7, and pDAB108718 with mean values 1.56, 0.07, 1.46, and 1.01 respectively.

FIGS. 47A, 47B, 47C, and 47D show exemplary protein expression results (V10) of YFP, AAD1, Cry34, and Cry35 respectively from the four constructs pDAB105748, pDAB105818, pDAB108717, and pDAB108718.

FIGS. 48A, 48B, 48C, and 48D show statistical analysis of protein expression results (V10) of YFP, AAD1, Cry34, and Cry35 respectively from the four constructs pDAB105748, pDAB105818, pDAB108717, and pDAB108718,

FIGS. 49A, 49B, 49C, and 49D show exemplary protein expression results (R3) of YFP, AAD1, Cry34, and Cry35 respectively from the four constructs pDAB105748, pDAB105818, pDAB108717, and pDAB108718.

FIGS. 50A, 50B, 50C, and 50D show statistical analysis of protein expression results (R3) of YFP, AAD1, Cry34, and Cry35 respectively from the four constructs pDAB105748, pDAB105818, pDAB108717, and pDAB108718.

FIG. 51 shows exemplary results of Western blot for protein expression of Cry34, Cry35, and AAD1 from pDAB108718 and pDAB108717.

The results show that all four transgenes in the single promoter-driven constructs are functional with good expression levels. Three genes (Cry34/Cry35/AAD1) in Ubi1 bidirectional stack show robust expression levels as similar to expression levels provided by the single Ubi1-driven gene stack (DExT).

While a number of exemplary aspects and embodiments have been discussed above, those of skill in the art will recognize certain modifications, permutations, additions and sub-combinations thereof It is therefore intended that the following appended claims and claims hereafter introduced are interpreted to include all such modifications, permutations, additions and sub-combinations as are within their true spirit and scope.

We claim:

1. A synthetic polynucleotide comprising a bi-directional promoter comprising:
(c) a first minimal core promoter element selected from SEQ ID NOs: 1 and 15-39;
(d) a second minimal core promoter element selected from SEQ ID NOs: 1 and 15-39; wherein the two minimal core promoter elements are in reverse complementary orientation with respect to each other in the synthetic polynucleotide; and
(e) a heterologous nucleotide sequence of interest operably linked to the bi-directional promoter.

2. The synthetic polynucleotide of claim 1, further comprising a different heterologous nucleotide sequence of interest operably linked to the bi-directional promoter.

3. The synthetic polynucleotide of claim 1, further comprising an exon from an Ubiquitin-1 gene and an intron from an Ubiquitin-1 gene.

4. The synthetic polynucleotide of claim 1, further comprising an upstream regulatory sequence from an Ubiquitin-1 gene.

5. The synthetic polynucleotide of claim 1, further comprising an element selected from the group consisting of an upstream regulatory sequence (URS), an enhancer element, an exon, an intron, a transcription start site, a TATA box, a heat shock consensus element, and combinations thereof.

6. A method for producing a transgenic cell, the method comprising:
transforming the cell with the synthetic polynucleotide of claim 1.

7. A plant cell comprising the synthetic polynucleotide of claim 1.

8. A nucleic acid construct for expressing multiple genes in plant cells and/or tissues, comprising,
(c) a bi-directional promoter; and
(d) two gene expression cassettes on opposite ends of the bi-directional promoter; wherein at least one of the gene expression cassettes comprises two or more genes linked via a translation switch selected from the group consisting of:
(v) a polynucleotide sequence coding a 2A peptide or 2A-like peptide selected from SEQ ID NOs: 52-55;
(vi) an internal ribosome entry site (IRES) selected from SEQ ID NOs: 56-61;
(vii) a polynucleotide sequence encoding an intein peptide selected from SEQ ID NOs: 62-77; and
(viii) a polynucleotide sequence encoding a linker peptide selected from SEQ ID NOs: 78-79.

9. The nucleic acid construct of claim 8, wherein the bi-directional promoter comprises:
(a) a first minimal core promoter element selected from SEQ ID NOs: 1 and 15-39; and
(b) a second minimal core promoter element selected from SEQ ID NOs: 1 and 15-39; wherein the two minimal core promoter elements are in reverse complementary orientation with respect to each other in the synthetic polynucleotide.

10. A method for generating a transgenic plant, comprising transforming a plant cell with the nucleic acid construct of claim 9.

11. A method for generating a transgenic cell, comprising transforming the cell with the nucleic acid construct of claim 9.

12. A plant cell comprising the nucleic acid construct of claim 9.

13. The plant cell of claim 12, wherein the nucleic acid construct is stably transformed into the plant cell.

14. A transgenic plant comprising the nucleic acid construct of claim 9.

15. A method for expressing multiple genes in plant cells and/or tissues, comprising introducing into the plant cells and/or tissues the nucleic acid construct of claim 9.
